SEQUENCE LISTING (26 sequences)

SEQ ID NO: 1 - DNA, 54 bp, Homo sapiens
tctgtaagagcagatccctggacaggcaaggaatacagagggcagcagacatcg

SEQ ID NO: 2 - DNA, 27 bp, Artificial Sequence
  Synthetic oligonucleotide sequence
gcctgtccagggatctgctcttacaga

SEQ ID NO: 3 - DNA, 22 bp, Artificial Sequence
  Synthetic oligonucleotide sequence
ggacaaaatacctgtattcctt

SEQ ID NO: 4 - DNA, 41 bp, Artificial Sequence
  Synthetic oligonucleotide sequence
  modified_base 41: n=dideoxyG
gatccctggacaggccggaagcggcttttttgccgcttccn

SEQ ID NO: 5 - DNA, 41 bp, Artificial Sequence
  Synthetic oligonucleotide sequence
gtgccgagacgttttttcgtctcggcactaggaatacaggt

SEQ ID NO: 6 - DNA, 26 bp, Artificial Sequence
  Synthetic oligonucleotide sequence
ctcccgcagacaccttctccttcaag

SEQ ID NO: 7 - DNA, 15 bp, Artificial Sequence
  Synthetic oligonucleotide sequence
tgatgatgaaatcgg

SEQ ID NO: 8 - DNA, 15 bp, Artificial Sequence
  Synthetic oligonucleotide sequence
tgatgatgaaatcga

SEQ ID NO: 9 - DNA, 39 bp, Artificial Sequence
  Synthetic oligonucleotide sequence
  modified_base 39: n=dideoxyG
ggtgtctgcgggagcggaagcggctttttgccgcttccn

SEQ ID NO: 10 - DNA, 37 bp, Artificial Sequence
  Synthetic oligonucleotide sequence
gctgcaccgcttttttgcggtgcacccgatttcatca

SEQ ID NO: 11 - DNA, 37 bp, Artificial Sequence
  Synthetic oligonucleotide sequence
gctgcaccgcttttttgcggtgcactcgatttcatca

SEQ ID NO: 12 - DNA, 96 bp, Artificial Sequence
  RC probe
gcacctcaaagctgttccgtcccagttgactatcctcagtgaattctagctactggcaatctgatccctatagtgagtcgtattacaggcacaaac

SEQ ID NO: 13 - DNA, 15 bp, Artificial Sequence
  Synthetic oligonucleotide sequence
agctactggcaatct

SEQ ID NO: 14 - DNA, 20 bp, Artificial Sequence
  T7 promoter
ccctatagtgagtcgtatta

SEQ ID NO: 15 - DNA, 6 bp, Artificial Sequence
  Eco RI site
gaattc

SEQ ID NO: 16 - DNA, 3 bp, Artificial Sequence
  Synthetic oligonucleotide sequence
gat

SEQ ID NO: 17 - DNA, 13 bp, Artificial Sequence
  Synthetic oligonucleotide sequence
caggcacaaacac

SEQ ID NO: 18 - DNA, 24 bp, Artificial Sequence
  Synthetic oligonucleotide sequence
gcacctcaaagctgttccgtccca

SEQ ID NO: 19 - DNA, 21 bp, Artificial Sequence
  RC primer
gataggagtcacttaagatcg

SEQ ID NO: 20 - DNA, 34 bp, Homo sapiens
ctaatctgtaagagcagatccctggacaggcaag

SEQ ID NO: 21 - DNA, 22 bp, Homo sapiens
aaggaatacaggtattttgtcc

SEQ ID NO: 22 - DNA, 14 bp, Artificial Sequence
  F5 1698 T7 Probe
gcctgtccagggat

SEQ ID NO: 23 - DNA, 37 bp, Artificial Sequence
  Synthetic oligonucleotide sequence
  modified_base 37: n=dideoxyG
cctggacaggccggaagcggcttttttgccgcttccn

SEQ ID NO: 24 - DNA, 25 bp, Homo sapiens
aaggaatacaggtattttgtccttg

SEQ ID NO: 25 - DNA, 25 bp, Artificial Sequence
  Synthetic oligonucleotide sequence
caaggacaaaatacctgtattcctt

SEQ ID NO: 26 - DNA, 40 bp, Artificial Sequence
  Synthetic oligonucleotide sequence
gtgccgagacgttttttcgtctcggcacaaggaatacagg

(19) United States
(12) Patent Application Publication
(10) Pub. No.: US 2007/0009894 A1
(43) Pub. Date: Jan. 11, 2007

(54) Oligonucleotide sequestering agents and method of use

(76) Inventor: Donald M. Crothers, Surrey Drive, Northford, CT (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/569,766

(22) PCT Filed: Aug. 23, 2004
(86) PCT No.: PCT/US04/27412

§ 371 Date: Jun. 30, 2006

(30) Foreign Application Priority Data
Aug. 25, 2003 (US) 60497821

(51) Int. Cl. C12Q 1/68

(52) U.S. Cl. 435/6

(57) ABSTRACT

The present application provides methods and compositions for use in detecting the presence of a target nucleic acid in a sample. In some embodiments, the methods employ sequestering agents which specifically interact with complementary nucleic acids which will be ligated together if the target nucleic acid is present in the sample. Detection of a ligation product comprising the complementary nucleic acids indicates that the target nucleic acid is present in the sample.

### FIELD OF THE INVENTION

The present invention relates to methods and reagents for detecting the presence of a target nucleic acid in a sample which comprises the target nucleic acid and non-target nucleic acids. Two nucleic acids which are complementary to nucleotide sequences in the target nucleic acid are provided. One or both of these complementary nucleic acids are contacted with a sequestering agent with which they specifically interact. The sequestering agent(s) reduce the likelihood that the complementary nucleic acids will hybridize to the non-target nucleic acids. In addition, in a preferred embodiment, the sequestering agents block access to the reactive groups for ligation on the 3′ and 5′ ends of the complementary nucleic acids, thereby reducing non-templated ligation (ligation of complementary nucleic acids which are not hybridized to the target nucleic acid). The two complementary nucleic acids are hybridized to their opposite strands in the target nucleic acid and ligated to one another. The presence of the ligation product is detected, thereby indicating the presence of the target nucleic acid in the sample.

The methods and reagents provided herein may be used to detect the presence of any desired target nucleic acid. In some embodiments, the methods and reagents are used to detect variant sequences, including single nucleotide polymorphisms (SNPs), allelic variants, and splice variants. In additional embodiments, the ligation product may be detected using ruthenium amperometry to detect hybridization of the ligation products themselves or other nucleic acids generated from the ligation products, such as amplification products or tagged DNA or RNA molecules, to detection probes immobilized on a universal detector. In some embodiments, the universal detector is a universal chip having gold or carbon electrodes.

### BACKGROUND OF THE INVENTION

Hybridization of polynucleotides to other polynucleotides having at least a portion of complementary nucleotide sequence by Watson-Crick base pairing is a fundamental process useful in a wide variety of research, medical, and industrial applications. Detecting the hybridization of a probe to a polynucleotide containing a target sequence is useful for gene expression analysis, DNA sequencing, and genomic analysis. Particular uses include identification of disease-related polynucleotides in diagnostic assays, screening for novel target polynucleotides in a sample, identification of specific target polynucleotides in mixtures of polynucleotides, identification of variant sequences, genotyping, amplification of specific target polynucleotides, and therapeutic blocking of inappropriately expressed genes, e.g. as described in Sambrook et al., Molecular Cloning: A Laboratory Manual, 2^{nd} Edition (Cold Spring Harbor Laboratory, New York, 1989); Keller and Manak, DNA Probes, 2^{nd} Edition (Stockton Press, New York, 1993); Milligan et al., 1993, J Med Chem, 36: 1923-1937; Drmanac et al., 1993. Science, 260: 1649-1652; Bains, 1993, J DNA Seq Map, 4:143-150.

Immobilized probes are useful for detecting polynucleotides containing a target nucleotide sequence, where each immobilized probe is functionally connected to a support and the hybridization of a polynucleotide to the immobilized probe can be detected. Most commonly, DNA probes are used to detect polynucleotides containing a target nucleotide sequence complementary to the probe sequence. The support for immobilized probes may be a flat surface, often called a “chip,” or the support may be the surface of a bead or other particle. Probes are usually immobilized in a known arrangement, or array, which provides a medium for matching known and unknown polynucleotides based on base-pairing rules. Preferably, the process of identifying the unknowns identified using a probe array is automated. Microarrays having a large of number of immobilized probes of known identity are used to determine complementary binding, allowing massively parallel studies of gene expression and gene discovery. For example, an experiment with a single DNA chip can provide researchers information on thousands of genes simultaneously. For example, Hashimoto et al. disclose an array of immobilized single-stranded probes wherein at least one probe has a nucleotide sequence complementary to the target gene(s) to be detected, such that each probe is immobilized onto the surface of an electrode or the tip of an optical fiber and an electrochemically or optically active substance capable of binding to double-stranded nucleic acid is used to detect hybridization of target genes to complementary immobilized probes (U.S. Pat. Nos. 5,776,672 and 5,972,692).

Universal Chips

Under some circumstances, a drawback to chip technology is that each chip must be manufactured specifically for the sequences to be detected, with a set of immobilized probes that are designed to be complementary to specific sequences to be detected. Chips specific for a single organism require a large manufacturing investment, and the chips can only be used for a narrowly defined range of samples. In contrast, a “universal chip” or “universal array” is organism-independent because the probes are not targeted to organism-specific sequences or products. Chips specific for a specific tissue, physiological condition, or developmental stage, often used for gene expression analysis, can likewise require a substantial manufacturing investment for use with a limited range of samples. A universal chip provides an unrestricted approach to studying tissues, physiological conditions, or developmental stages of interest. Manufacturing quality control can be improved by using a universal chip for polynucleotide detection.

One approach to universal chip design involves attaching a set of oligonucleotide probes to a chip surface, where the set of oligonucleotide probes includes all possible sequences of oligonucleotides that are 5, 6, 7, 8, 9, 10 or more nucleotides in length. The probes needed for these arrays can be designed using a simple combinatorial algorithm. The chip is incubated with a mixture that may contain DNA, cDNA, RNA or other hybridizable material, and hybridization to each probe of known sequence is measured. However, the specificity of such an array may be impaired because different sequences may have different requirements for stringent hybridization. In addition, such a universal array does not prevent false positives resulting from frameshifting where, for example in a universal array having probes that are six nucleotides long, the final four nucleotides of a sample polynucleotide may hybridize to the complementary final four nucleotides of a six-nucleotide probe, but the same sample polynucleotide would not hybridize to the entire six-nucleotide probe sequence.

Suyama et al. (2000, Curr Comp Mol Biol 7:12-13) disclose a universal chip system for gene expression profiling of a sample, where the chip system utilizes “DNA computing” instead of binding of transcripts to probes. The DNA computing system of Suyama et al. indirectly determines which transcripts are present by measuring binding of coded adapters to a universal set of immobilized probes on the universal chip. Only those coded adapters with a region complementary to a region of a transcript present in a sample will undergo the subsequent manipulations and the processing steps that generate adapters capable of binding to probes on the universal chip.

Tags

An alternative approach to manufacturing a universal chip involves using a set of tag sequences that do not naturally occur in the target polynucleotides, where the tags bind to complementary probes on a universal chip. Tags for such uses are sometimes known as “address tags” or “zip codes” or are considered to be analogous to “bar codes” for identifying targets. Detection, identification, tracking, sorting, retrieving or other manipulations are then directed at tag sequences and not the sequences of the target polynucleotides. Oligonucleotide tags may be covalently attached to or incorporated into polynucleotides. Tags may become associated with a polynucleotide by hybridization of a separate oligonucleotide which functions as a linker by virtue of having at least two domains, one with tag sequence complementary to a probe and one with sequence complementary to at least a portion of the target polynucleotide. Systems employing oligonucleotide tags have been proposed as means for manipulating and identifying individual molecules in complex mixtures, for example to detect polynucleotides having target nucleotide sequences, or as an aid to screening genomic, cDNA, or combinatorial libraries for drug candidates. Brenner and Lerner, 1992, Proc Natl Acad Sci, 89: 5381-5383; Alper, 1994, Science, 264: 1399-1401; Needels et al., 1993, Proc Nat Acad Sci, 90: 10700-10704.

Spurious signals

The usefulness of tagged polynucleotides depends in large part on success in achieving specific hybridization between a tag and its complementary probe immobilized to a surface. For an oligonucleotide tag to successfully identify a polynucleotide, the number of false positive and false negative signals must be minimized. Unfortunately, spurious signals are not uncommon because base pairing and base stacking free energies can vary widely among nucleotide sequences in a duplex or triplex structure. For example, a tag-probe duplex having a different number of guanosine-cytosine (G-C) pairs than another duplex will have a different melting temperature, such that tag-probe duplexes with differing G-C ratios will have different stringency requirements for hybridization. In addition, a tag-probe duplex consisting of a repeated sequence of adenosine (A) and thymidine (T) bound to its complement may have less stability than a duplex of equal length consisting of a repeated sequence of G and C bound to a partially complementary target containing a mismatch, due to differences in stacking energy. Special reagents are often required to balance these differences in stacking energy.

Spurious signals can also result from “frameshifting” as described above. This problem has been addressed by employing a “comma-less” code, which ensures that a probe out of register (frameshifted) with respect to its complementary tag would result in a duplex with one or more mismatches for each of its codons, which forms an unstable duplex.

In view of the above problems with spurious signals, researchers have developed various oligonucleotide-based tagging systems which provide a sufficient repertoire of tags, but which also minimize the occurrence of false positive and false negative signals without the need to employ special reagents for altering natural base pairing and base stacking free energy differences, or elaborate encoding systems for comma-less codes. Such tagging systems find applications in many areas, including construction and use of combinatorial chemical libraries, large-scale mapping and sequencing of DNA, genetic identification, and medical diagnostics.

Brenner et al. disclose a “universal” chip system that attaches tags to the ends of polynucleotide fragments through reactive moieties, where spurious signals are avoided by designing a repertoire of multi-subunit oligonucleotide tags with sequences such that the stability of any mismatched duplex or triplex between a tag and a complement to another tag is far lower than that of any perfectly matched duplex between the tag and its own complement. U.S. Pat. Nos. 5,604,097, 5,654,413, 5,846,719, 5,863,722, 6,140,489, 6,150,516, 6,172,214, 6,172,218, 6,352,828, 6,235,475. Morris et al. (U.S. Pat. No. 6,458,530, EP 0799897) disclose the use of tags and arrays of complementary probes to label and track compositions including cells and viruses, and to facilitate analysis of cell and viral phenotypes.

An alternate approach involves multicomponent tagging systems where tags are not attached to polynucleotides but rather, are found on separate components that are hybridized to the polynucleotides in order to adapt, index, and/or detect polynucleotides having a defined nucleotide sequence. The method disclosed in U.S. Pat. No. 6,261,782 and related patents and applications (Lizardi et al.) permits the user to sort and identify target polynucleotides in a sample by generating “sticky ends” using nucleic acid cleaving reagents, indexing the cleaved polynucleotide fragments into sets by adding adapter-indexer oligonucleotides with ends complementary to various sticky ends to the sample, adding ligator-detector oligonucleotides with sticky ends complementary to the sticky ends of adapter-indexers, hybridizing the entire sample with a plurality of detector probes, covalently coupling the ligator-detectors to the detector probes, and finally detecting coupling of ligator-detectors to the detector probes.

Another multicomponent system is disclosed by Balch et al., in U.S. Pat. No. 6,331,441, using a bifunctional linker with a domain that hybridizes to an immobilized capture probe in a universal array and a domain that hybridizes to an analyte containing a target. Balch et al. also discloses amplification of a target polynucleotide to generate amplicons containing both target sequence and a unique universal sequence complementary to a capture probe, where the unique universal sequence may be introduced through PCR or LCR primers (U.S. Pat. No. 6,331,441).

Any hybridization-based method for detecting the presence of a target nucleic acid in a sample, whether or not it utilizes universal chips or tags, is dependent on the initial hybridization of one or more nucleic acids to the target nucleic acid in the sample. Accordingly, it is beneficial to reduce the occurrence of false positives which result from hybridization to non-target nucleic acids in the sample. One mechanism for discriminating between target nucleic acids and non-target nucleic acids is to conduct the hybridization at stringent temperatures which reduce the extent of hybridization to non-target nucleic acids. However, it may be difficult to maintain such stringent conditions. In addition, where a large number of nucleic acids are to be simultaneously hybridized to different target nucleic acids in a single hybridization reaction, it may be difficult to identify stringent temperatures which allow all of the nucleic acids to specifically hybridize to their target nucleic acids. Conducting the hybridization at too great a stringency may prevent some of the nucleic acids from efficiently hybridizing to their target nucleic acids while conducting the hybridization at too low a stringency may lead to false positives resulting from hybridization of some of the nucleic acids to non-target nucleic acids present in the sample. The present invention allows hybridization-based detection methods to be performed without conducting the hybridization at stringent temperatures.

### SUMMARY OF THE INVENTION

Some embodiments of the present invention are described in the following numbered paragraphs:

1. A method for detecting the presence of a target nucleic acid in a sample comprising:
providing a sample comprising a target nucleic acid and non-target nucleic acids;
providing a first complementary nucleic acid which is complementary to a first nucleotide sequence in a target nucleic acid;
providing a second complementary nucleic acid which is complementary to a second nucleotide sequence in said target nucleic acid;
providing a first sequestering agent which specifically interacts with said first complementary nucleic acid, wherein said first sequestering agent reduces the likelihood that said first complementary nucleic acid will hybridize to said non-target nucleic acids;
hybridizing said first complementary nucleic acid to said first nucleotide sequence in said target nucleic acid;
hybridizing said second complementary nucleic acid to said second nucleotide sequence in said target nucleic acid;
ligating said first complementary nucleic acid which is hybridized to said first nucleotide sequence in said target nucleic acid to said second complementary nucleic acid which is hybridized to said second nucleotide sequence in said target nucleic acid, thereby forming a ligation product; and
detecting the presence of said ligation product.

2. The method of Paragraph 1, wherein said first complementary nucleic acid and said second complementary nucleic acid are present on the same nucleic acid molecule such that said ligation product comprises a circular nucleic acid.

3. The method of Paragraph 1, wherein a first oligonucleotide comprises said first complementary nucleic acid and a second distinct oligonucleotide comprises said second complementary nucleic acid.

4. The method of Paragraph 1, wherein said first nucleotide sequence in said target nucleic acid and said second nucleotide sequence in said target nucleic acid are immediately adjacent to one another on said target nucleic acid.

5. The method of Paragraph 1, wherein said first nucleotide sequence in said target nucleic acid and said second nucleotide sequence in said target nucleic acid are not immediately adjacent to one another on said target nucleic acid.

6. The method of Paragraph 1, further comprising providing a second sequestering agent which interacts with said second complementary nucleic acid, thereby reducing the likelihood that said second oligonucleotide will hybridize to said non-target nucleic acids.

7. The method of Paragraph 1, wherein said first sequestering agent comprises a nucleic acid comprising a nucleotide sequence which is complementary to at least a portion of said first complementary nucleic acid, wherein a duplex between said first complementary nucleic acid and said sequestering agent has a melting point intermediate to the melting point of a duplex between said first complementary nucleic acid and said non-target nucleic acids and the melting point of a duplex between said first complementary nucleic acid and said first nucleotide sequence in said target nucleic acid.

8. The method of Paragraph 7, wherein the residue at the 3′ terminus of said first sequestering agent lacks a 3-hydroxyl group such that the 5′ phosphate of said first complementary nucleic acid cannot be ligated to the 3′ terminus of said sequestering agent.

9. The method of Paragraph 8, wherein the residue at the 3′ terminus of said first sequestering agent comprises a dideoxysugar.

10. The method of Paragraph 8, wherein the residue at the 3′ terminus of said sequestering agent comprises a blocking group such that the 5′ phosphate of said first complementary nucleic acid cannot be ligated to the 3′ terminus of said first sequestering agent.

11. The method of Paragraph 10, wherein said blocking group is selected from the group consisting of NH₂, F, Cl, Br, NO₂, OR₁, O—C(O)—R₂, NHR₃, alkyl, and H where R₁ is selected from the group consisting of carbonyl, alkyl, acyl, alkene, or heteroalkyl, said alkyl, acyl, alkene or heteroalkyl group being substituted or unsubstituted, R₂ is selected from the group consisting of alkyl, acyl, alkene, or heteroalkyl, said alkyl, acyl, alkene or heteroalkyl group being substituted or unsubstituted and R3 is selected from the group consisting of alkyl, acyl, alkene, or heteroalkyl, said alkyl, acyl, alkene or heteroalkyl group being substituted or unsubstituted.

12. The method of Paragraph 8, wherein said sequestering agent comprises a stem loop.

13. The method of Paragraph 12, wherein the 3′ terminus is in the duplex portion of said stem loop.

14. The method of Paragraph 7, wherein said sequestering agent has a linear conformation.

15. The method of Paragraph 7, wherein said sequestering agent reduces the accessibility of the 5′ phosphate of said first complementary nucleic acid to a ligase when said first complementary nucleic acid is hybridized to said sequestering agent.

16. The method of Paragraph 7, wherein said sequestering agent lacks a 5′ phosphate.

17. The method of Paragraph 16, wherein the nucleotide at the 5′ terminus of said sequestering agent comprises a blocking group which prevents said nucleotide from being ligated to another nucleic acid, wherein said blocking group is selected from the group consisting of NH₂, F, Cl, Br, NO₂, OR₁, O—C(O)—R₂, NHR₃, alkyl, and H where R₁ is selected from the group consisting of carbonyl, alkyl, acyl, alkene, or heteroalkyl, said alkyl, acyl, alkene or heteroalkyl group being substituted or unsubstituted, R₂ is selected from the group consisting of alkyl, acyl, alkene, or heteroalkyl, said alkyl, acyl, alkene or heteroalkyl group being substituted or unsubstituted and R3 is selected from the group consisting of alkyl, acyl, alkene, or heteroalkyl, said alkyl, acyl, alkene or heteroalkyl group being substituted or unsubstituted.

18. The method of Paragraph 7, wherein said sequestering agent reduces the accessibility of the 3′ hydroxyl of said first complementary nucleic acid to a ligase when said first complementary nucleic acid is hybridized to said sequestering agent.

19. The method of Paragraph 6, wherein said first equestering agent reduces the accessibility of the 5′ phosphate of said first complementary nucleic acid to a ligase when said first complementary nucleic acid is hybridized to said first sequestering agent and wherein said second sequestering agent reduces the accessibility of the 3′ hydroxyl of said second complementary nucleic acid to a ligase when said second complementary nucleic acid is hybridized to said second sequestering agent.

20. The method of Paragraph 1, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid.

21. The method of Paragraph 6 wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid and wherein said hybridizing step comprises hybridizing said second complementary nucleic acid to said second nucleotide sequence in said target nucleic acid at a temperature selected from the group consisting of a temperature at least 5 degrees Celsius below the melting temperature of the duplex between said second complementary nucleic acid and said second nucleotide sequence in said target nucleic acid, a temperature at least 10 degrees Celsius below the melting temperature of the duplex between said second complementary nucleic acid and said second nucleotide sequence in said target nucleic acid, a temperature at least 15 degrees Celsius below the melting temperature of the duplex between said second complementary nucleic acid and said second nucleotide sequence in said target nucleic acid, a temperature at least 20 degrees Celsius below the melting temperature of the duplex between said second complementary nucleic acid and said second nucleotide sequence in said target nucleic acid, and a temperature at least 25 degrees Celsius below the melting temperature of the duplex between said second complementary nucleic acid and said second nucleotide sequence in said target nucleic acid.

22. The method of Paragraph 7, wherein said target nucleic acid comprises a single nucleotide polymorphism.

23. The method of Paragraph 22, wherein said first complementary nucleic acid comprises the variable nucleotide of said single nucleotide polymorphism such that said first complementary nucleic acid is fully complementary to a first allele of said single nucleotide polymorphism.

24. The method of Paragraph 23, wherein said variable nucleotide position is at the 3′ terminus of said first complementary nucleic acid.

25. The method of Paragraph 23, further comprising:
providing a third complementary nucleic acid wherein said third complementary nucleic acid comprises the variable nucleotide of said single nucleotide polymorphism such that said third complementary nucleic acid is fully complementary to a second allele of said single nucleotide polymorphism,
providing a second sequestering agent comprising a nucleic acid which is complementary to at least a portion of said third complementary nucleic acid and wherein said at least a portion of said third complementary nucleic acid includes said variable nucleotide of said second allele of said single nucleotide polymorphism;
wherein said at least a portion of said first complementary nucleic acid to which said first sequestering agent is complementary includes said variable nucleotide of said first allele of said single nucleotide polymorphism.

26. The method of Paragraph 1, wherein said sequestering agent is present in excess of said first complementary nucleic acid.

27. The method of Paragraph 26, wherein said sequestering agent is present in a molar excess of about 1.5 to about 2 relative to said first complementary nucleic acid.

28. The method of Paragraph 1, wherein the concentrations of said first complementary nucleic acid and said sequestering agent are selected so that at equilibrium about 5% or less of said first nucleotide sequence in said target nucleic acid is unoccupied by said first complementary nucleic acid.

29. The method of Paragraph 1, wherein said sample comprises genomic DNA.

30. The method of Paragraph 1, wherein said step of detecting the presence of said ligation product comprises performing a PCR reaction to amplify said ligation product, thereby generating an amplified ligation product and detecting said amplified ligation product.

31. The method of Paragraph 1, wherein said step of detecting the presence of said ligation product comprises circularizing a nucleic acid template, thereby generating a circular molecule if said ligation product is present and detecting said circular molecule.

32. The method of Paragraph 1, wherein said step of detecting the presence of said ligation product comprises performing a rolling circle amplification reaction, thereby generating an amplification product and detecting said amplification product.

33. The method of Paragraph 1, wherein said step of detecting the presence of said ligation product comprises detecting hybridization of said ligation product or of a nucleic acid indicative of the presence of said ligation product to a solid support.

34. The method of Paragraph 33, wherein said nucleic acid indicative of the presence of said ligation product comprises an identifier tag wherein said identifier tag is generated only when said target nucleotide sequence corresponding to said identifier tag is present in said sample.

35. The method of Paragraph 33, wherein said solid support comprises a universal detector.

36. The method of Paragraph 35, wherein said universal detector comprises detection probes coupled to a detecting component which measures hybridization of said ligation product or of said nucleic acid indicative of the presence of said ligation product to any of said detection probes.

37. The method of Paragraph 36, wherein said detecting component is electrochemical, fluorescent, colorimetric, radiometric or magnetic.

38. The method of Paragraph 37, wherein said detecting component is electrochemical.

39. The method of Paragraph 36, wherein said detection probes coupled to said detecting component are attached to a surface, film, or particle.

40. The method of Paragraph 39, wherein said detection probes are attached to said surface, film, or particle by covalent bonds, ionic bonds, electrostatic interactions or adsorptive interactions.

41. The method of Paragraph 39, wherein said detection probes are attached to a particle such as a bead.

42. The method of Paragraph 39, wherein said detection probes are attached to a plurality of particles.

43. The method of Paragraph 35, wherein said universal detector comprises an array of detection probes coupled to detecting components, said array comprising electrodes attached to a substrate.

44. The method of Paragraph 43, wherein said electrodes are gold or carbon.

45. The method of Paragraph 44, wherein said electrodes are gold.

46. The method of Paragraph 43, further comprising measuring hybridization to any said detection probe by ruthenium amperometry.

47. The method of Paragraph 43, wherein said electrodes are coated with protein which can be bound by oligonucleotides derivatized with a moiety that binds said protein that coats said electrode.

48. The method of Paragraph 47, wherein said electrodes are coated with avidin such that said electrodes can be bound by biotin-labeled oligonucleotides.

49. An isolated nucleic acid comprising:
a first nucleotide sequence complementary to a probe nucleic acid such that said probe nucleic acid can hybridize to said nucleic acid, wherein at least a portion of said probe nucleic acid is complementary to a target nucleic acid;
a second nucleotide sequence comprising a region which forms a structure such that one terminus of said nucleic acid is in proximity to one terminus of said probe nucleic acid when said probe nucleic acid is hybridized to said nucleic acid and wherein a duplex between said probe nucleic acid and said first nucleotide sequence has a melting point intermediate to the melting point of a duplex between said probe nucleic acid and said target nucleic acid and the melting point of a duplex between said probe nucleic acid and non-target nucleic acids.

50. The nucleic acid of Paragraph 49 wherein said nucleic acid reduces the accessibility of the 5′ phosphate of said probe nucleic acid to a ligase when said probe nucleic acid is hybridized to said nucleic acid.

51. The nucleic acid of Paragraph 49, wherein said nucleic acid lacks a 5′ phosphate.

52. The nucleic acid of

51, wherein the nucleotide at the 5′ terminus of said nucleic acid comprises a blocking group which prevents said nucleotide from being ligated to another nucleic acid, wherein said blocking group is selected from the group consisting of NH₂, F, Cl, Br, NO₂, OR₁, O—C(O)—R₂, NHR₃, alkyl, and H where R₁ is selected from the group consisting of carbonyl, alkyl, acyl, alkene, or heteroalkyl, said alkyl, acyl, alkene or heteroalkyl group being substituted or unsubstituted, R₂ is selected from the group consisting of alkyl, acyl, alkene, or heteroalkyl, said alkyl, acyl, alkene or heteroalkyl group being substituted or unsubstituted and R3 is selected from the group consisting of alkyl, acyl, alkene, or heteroalkyl, said alkyl, acyl, alkene or heteroalkyl group being substituted or unsubstituted.

53. The nucleic acid of Paragraph 49, wherein the terminus of said nucleic acid which is adjacent to said probe nucleic acid when said probe nucleic acid is hybridized to said nucleic acid contains a moiety thereon which cannot be ligated to said probe nucleic acid by a ligase.

54. The nucleic acid of Paragraph 53, wherein said moiety which cannot be ligated to said probe nucleic acid by a ligase is on the residue at the 3′ terminus of said nucleic acid.

55. The nucleic acid of Paragraph 54, wherein the 3′ terminus of said nucleic acid lacks a 3-hydroxyl group such that the 5′ phosphate of said probe nucleic acid cannot be ligated to the 3′ terminus of said nucleic acid.

56. The nucleic acid of Paragraph 55, wherein the residue at the 3′ terminus of said nucleic acid comprises a dideoxysugar.

57. The nucleic acid of Paragraph 55, wherein the residue at the 3′ terminus of said nucleic acid comprises a blocking group such that the 5′ phosphate of said probe nucleic acid cannot be ligated to the 3′ terminus of said nucleic acid.

58. The nucleic acid of Paragraph 57, wherein said blocking group is selected from the group consisting of NH₂, F, Cl, Br, NO₂, OR₁, O—C(O)—R₂, NHR₃, alkyl, and H where R₁ is selected from the group consisting of carbonyl, alkyl, acyl, alkene, or heteroalkyl, said alkyl, acyl, alkene or heteroalkyl group being substituted or unsubstituted, R₂ is selected from the group consisting of alkyl, acyl, alkene, or heteroalkyl, said alkyl, acyl, alkene or heteroalkyl group being substituted or unsubstituted and R3 is selected from the group consisting of alkyl, acyl, alkene, or heteroalkyl, said alkyl, acyl, alkene or heteroalkyl group being substituted or unsubstituted.

59. The nucleic acid of Paragraph 49, wherein said structure comprises a stem loop.

60. The nucleic acid of Paragraph 59, wherein the 3′ terminus of said nucleic acid is in the duplex portion of said stem loop.

61. The nucleic acid of Paragraph 49, wherein said first nucleotide sequence which is complementary to at least a portion of said probe nucleic acid comprises a sequence which is polymorphic in the genome of an organism containing said target nucleic acid.

62. The nucleic acid of Paragraph 61, wherein said first nucleotide sequence which is complementary to at least a portion of said probe nucleic acid comprises the variable nucleotide of a single nucleotide polymorphism in the genome of an organism containing said target nucleic acid.

63. The nucleic acid of Paragraph 62, wherein said variable nucleotide is complementary to the 3′ terminus of said probe nucleic acid.

64. A method for generating a ligation product comprising:
providing a first nucleic acid and a second nucleic acid;
forming sequestration complexes, said sequestration complexes comprising either said first nucleic acid or said second nucleic acid specifically complexed with a sequestering agent or comprising both said first nucleic acid and said second nucleic acid specifically complexed with sequestering agents;
hybridizing said first nucleic acid and said second nucleic acid to a third nucleic acid containing nucleotide sequences complementary to said first and second nucleic acids, wherein the stability of a duplex between said third nucleic acid and said first nucleic acid or said second nucleic acid is greater than the stability of said sequestration complexes; and
ligating said first nucleic acid to said second nucleic acid while said first nucleic acid and said second nucleic acid are hybridized to said third nucleic acid.

65. A method for determining whether a sample contains a target nucleic acid comprising:
providing a sample to be tested for the presence of a target nucleic acid, said sample containing non-target nucleic acids;
providing a first complementary nucleic acid which is complementary to a first nucleotide sequence in a target nucleic acid;
providing a second complementary nucleic acid which is complementary to a second nucleotide sequence in said target nucleic acid;
providing a first sequestering agent which specifically interacts with said first complementary nucleic acid, wherein said first sequestering agent reduces the likelihood that said first complementary nucleic acid will hybridize to said non-target nucleic acids;
performing a hybridization reaction such that said first complementary nucleic acid will hybridize to said first nucleotide sequence in said target nucleic acid if said target nucleic acid is present in said sample;
performing a hybridization reaction such that said second complementary nucleic acid will hybridize to said second nucleotide sequence in said target nucleic acid if said target nucleic acid is present in said sample;
providing a ligase such that if said first complementary nucleic acid is hybridized to said first nucleotide sequence in said target nucleic acid and said second complementary nucleic acid is hybridized to said second nucleotide sequence in said target nucleic acid, a ligation product will be generated; and
determining whether said ligation product has been generated.

66. A method for determining whether a target nucleic acid is present in a sample comprising:
providing a sample to be tested for the presence of a target nucleic acid said sample comprising non-target nucleic acids;
performing an amplification reaction on said sample, thereby generating an amplified target nucleic acid if said sample contains said target nucleic acid;
providing a probe comprising a first end comprising a first complementary nucleic acid complementary to a first nucleotide sequence in said target nucleic acid, a second end comprising a second complementary nucleic acid complementary to a second nucleotide sequence in said target nucleic acid, a promoter recognized by an RNA polymerase, and a nucleotide sequence complementary to a tag sequence indicative of the presence of said target nucleic acid in said sample;
providing a first sequestering agent which specifically interacts with said first complementary nucleic acid, wherein said first sequestering agent reduces the likelihood that said first complementary nucleic acid will hybridize to said non-target nucleic acids;
contacting said sample with said probe, such that said first complementary nucleic acid will hybridize to said first nucleotide sequence in said amplified target nucleic acid and said second complementary nucleic acid will hybridize to said second nucleotide sequence in said amplified target nucleic acid and said probe will adopt a circular conformation if said target nucleic acid is present in said sample;
ligating the ends of said circular probe together if said circular probe is present;
performing a transcription reaction with said RNA polymerase, thereby generating a transcript comprising said tag if said sample contains said target nucleic acid; and
determining whether said transcript has been generated.

67. The method of Paragraph 66, wherein said RNA polymerase is selected from the group consisting of T7 RNA polymerase, R4 RNA polymerase, T3 RNA polymerase, E. coli RNA polymerase, SP6 RNA polymerase, RNA polymerase II and RNA polymerase III.

68. The method of Paragraph 66, wherein said promoter is positioned 3′ of said first complementary nucleic acid and said nucleotide sequence complementary to said tag is positioned 5′ of said second complementary nucleic acid.

69. The method of Paragraph 66, further comprising performing an exonuclease digestion after ligating the ends of said probe together.

70. The method of Paragraph 66, further comprising providing a second sequestering agent which specifically interacts with said second complementary nucleic acid, wherein said second sequestering agent reduces the likelihood that said second complementary nucleic acid will hybridize to said non-target nucleic acids.

71. The method of Paragraph 66, wherein said first sequestering agent reduces the accessibility of the 5′ phosphate of said first complementary nucleic acid to a ligase when said first complementary nucleic acid is hybridized to said first sequestering agent.

72. The method of Paragraph 66, wherein said first sequestering agent reduces the accessibility of the 3′ hydroxyl of said first complementary nucleic acid to a ligase when said first complementary nucleic acid is hybridized to said sequestering agent.

73. The method of Paragraph 70, wherein said first sequestering agent reduces the accessibility of the 5′ phosphate of said first complementary nucleic acid to a ligase when said first complementary nucleic acid is hybridized to said first sequestering agent and said second sequestering agent reduces the accessibility of the 3′ hydroxyl of said second complementary nucleic acid to a ligase when said second complementary nucleic acid is hybridized to said second sequestering agent.

74. The method of Paragraph 66, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid.

75. The method of Paragraph 70 wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid and wherein said hybridizing step comprises hybridizing said second complementary nucleic acid to said second nucleotide sequence in said target nucleic acid at a temperature selected from the group consisting of a temperature at least 5 degrees Celsius below the melting temperature of the duplex between said second complementary nucleic acid and said second nucleotide sequence in said target nucleic acid, a temperature at least 10 degrees Celsius below the melting temperature of the duplex between said second complementary nucleic acid and said second nucleotide sequence in said target nucleic acid, a temperature at least 15 degrees Celsius below the melting temperature of the duplex between said second complementary nucleic acid and said second nucleotide sequence in said target nucleic acid, a temperature at least 20 degrees Celsius below the melting temperature of the duplex between said second complementary nucleic acid and said second nucleotide sequence in said target nucleic acid and a temperature at least 25 degrees Celsius below the melting temperature of the duplex between said second complementary nucleic acid and said second nucleotide sequence in said target nucleic acid.

76. A method for determining whether a target nucleic acid is present in a sample comprising:
providing a sample to be tested for the presence of a target nucleic acid said sample comprising non-target nucleic acids;
performing an amplification reaction on said sample, thereby generating an amplified target nucleic acid if said sample contains said target nucleic acid;
providing a probe comprising a first end comprising a first complementary nucleic acid complementary to a first nucleotide sequence in said target nucleic acid, a second end comprising a second complementary nucleic acid complementary to a second nucleotide sequence in said target nucleic acid, a promoter recognized by an RNA polymerase, and a nucleotide sequence complementary to a tag sequence indicative of the presence of said target nucleic acid in said sample;
providing a first sequestering agent which sepecifically interacts with said first complementary nucleic acid, wherein said first sequestering agent reduces the likelihood that said first complementary nucleic acid will hybridize to said non-target nucleic acids;
contacting said sample with said probe, such that said first complementary nucleic acid will hybridize to said first nucleotide sequence in said amplified target nucleic acid and said second complementary nucleic acid will hybridize to said second nucleotide sequence in said amplified target nucleic acid and said probe will adopt a circular conformation if said target nucleic acid is present in said sample;
ligating the ends of said circular probe together if said circular probe is present;
linearizing said circularized probe;
performing a transcription reaction with said RNA polymerase, thereby generating a transcript comprising said tag if said sample contains said target nucleic acid; and
determining whether said transcript has been generated

77. The method of Paragraph 76, wherein said RNA polymerase is selected from the group consisting of T7 RNA polymerase, R4 RNA polymerase, T3 RNA polymerase, E. Coli RNA polymerase, SP6 RNA polymerase, RNA polymerase II and RNA polymerase III.

78. The method of Paragraph 76, wherein said promoter is positioned 3′ of said first complementary nucleic acid and said nucleotide sequence complementary to said tag is positioned 5′ of said second complementary nucleic acid.

79. The method of Paragraph 76, further comprising performing an exonuclease digestion after ligating the ends of said probe together.

80. The method of Paragraph 76, further comprising providing a second sequestering agent which specifically interacts with said second complementary nucleic acid, wherein said second sequestering agent reduces the likelihood that said second complementary nucleic acid will hybridize to said non-target nucleic acids.

81. The method of Paragraph 76, wherein said first sequestering agent reduces the accessibility of the 5′ phosphate of said first complementary nucleic acid to a ligase when said first complementary nucleic acid is hybridized to said first sequestering agent.

82. The method of Paragraph 76, wherein said first sequestering agent reduces the accessibility of the 3′ hydroxyl of said first complementary nucleic acid to a ligase when said first complementary nucleic acid is hybridized to said sequestering agent.

83. The method of Paragraph 80, wherein said first sequestering agent reduces the accessibility of the 5′ phosphate of said first complementary nucleic acid to a ligase when said first complementary nucleic acid is hybridized to said first sequestering agent and said second sequestering agent reduces the accessibility of the 3′ hydroxyl of said second complementary nucleic acid to a ligase when said second complementary nucleic acid is hybridized to said second sequestering agent.

84. The method of Paragraph 76, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid.

85. The method of Paragraph 80 wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid and wherein said hybridizing step comprises hybridizing said second complementary nucleic acid to said second nucleotide sequence in said target nucleic acid at a temperature selected from the group consisting of a temperature at least 5 degrees Celsius below the melting temperature of the duplex between said second complementary nucleic acid and said second nucleotide sequence in said target nucleic acid, a temperature at least 10 degrees Celsius below the melting temperature of the duplex between said second complementary nucleic acid and said second nucleotide sequence in said target nucleic acid, a temperature at least 15 degrees Celsius below the melting temperature of the duplex between said second complementary nucleic acid and said second nucleotide sequence in said target nucleic acid, a temperature at least 20 degrees Celsius below the melting temperature of the duplex between said second complementary nucleic acid and said second nucleotide sequence in said target nucleic acid and a temperature at least 25 degrees Celsius below the melting temperature of the duplex between said second complementary nucleic acid and said second nucleotide sequence in said target nucleic acid.

86. A method for determining whether a target nucleic acid is present in a sample comprising:
providing a sample to be tested for the presence of a target nucleic acid said sample comprising non-target nucleic acids;
performing an amplification reaction on said sample, thereby generating an amplified target nucleic acid if said sample contains said target nucleic acid;
providing a probe comprising a first end comprising a first complementary nucleic acid complementary to a first nucleotide sequence in said target nucleic acid, a second end comprising a second complementary nucleic acid complementary to a second nucleotide sequence in said target nucleic acid, and a nucleotide sequence comprising a sequence complementary to or identical to a tag sequence indicative of the presence of said target nucleic acid in said sample;
providing a first sequestering agent which specifically interacts with said first complementary nucleic acid, wherein said first sequestering agent reduces the likelihood that said first complementary nucleic acid will hybridize to said non-target nucleic acids;
contacting said sample with said probe, such that said first complementary nucleic acid will hybridize to said first nucleotide sequence in said amplified target nucleic acid and said second complementary nucleic acid will hybridize to said second nucleotide sequence in said amplified target nucleic acid and said probe will adopt a circular conformation if said target nucleic acid is present in said sample;
ligating the ends of said circular probe together if said circular probe is present;
linearizing said circularized probe; and
determining whether said linearized probe has been generated.

87. The method of Paragraph 86, wherein said nucleotide sequence comprising said tag is positioned 5′ of said second complementary nucleic acid.

88. The method of Paragraph 86, further comprising performing an exonuclease digestion after ligating the ends of said probe together.

89. The method of Paragraph 86, further comprising providing a second sequestering agent which specifically interacts with said second complementary nucleic acid, wherein said second sequestering agent reduces the likelihood that said second complementary nucleic acid will hybridize to said non-target nucleic acids.

90. The method of Paragraph 86, wherein said first sequestering agent reduces the accessibility of the 5′ phosphate of said first complementary nucleic acid to a ligase when said first complementary nucleic acid is hybridized to said first sequestering agent.

91. The method of Paragraph 86, wherein said first sequestering agent reduces the accessibility of the 3′ hydroxyl of said first complementary nucleic acid to a ligase when said first complementary nucleic acid is hybridized to said sequestering agent.

92. The method of Paragraph 89, wherein said first sequestering agent reduces the accessibility of the 5′ phosphate of said first complementary nucleic acid to a ligase when said first complementary nucleic acid is hybridized to said first sequestering agent and said second sequestering agent reduces the accessibility of the 3′ hydroxyl of said second complementary nucleic acid to a ligase when said second complementary nucleic acid is hybridized to said second sequestering agent.

93. The method of Paragraph 86, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid.

94. The method of Paragraph 89 wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid and wherein said hybridizing step comprises hybridizing said second complementary nucleic acid to said second nucleotide sequence in said target nucleic acid at a temperature selected from the group consisting of a temperature at least 5 degrees Celsius below the melting temperature of the duplex between said second complementary nucleic acid and said second nucleotide sequence in said target nucleic acid, a temperature at least 10 degrees Celsius below the melting temperature of the duplex between said second complementary nucleic acid and said second nucleotide sequence in said target nucleic acid, a temperature at least 15 degrees Celsius below the melting temperature of the duplex between said second complementary nucleic acid and said second nucleotide sequence in said target nucleic acid, a temperature at least 20 degrees Celsius below the melting temperature of the duplex between said second complementary nucleic acid and said second nucleotide sequence in said target nucleic acid and a temperature at least 25 degrees Celsius below the melting temperature of the duplex between said second complementary nucleic acid and said second nucleotide sequence in said target nucleic acid.

95. A method for determining whether a target nucleic acid is present in a sample comprising:
providing a sample to be tested for the presence of a target nucleic acid said sample comprising non-target nucleic acids;
providing a probe comprising a first end comprising a first complementary nucleic acid complementary to a first nucleotide sequence in said target nucleic acid, a second end comprising a second complementary nucleic acid complementary to a second nucleotide sequence in said target nucleic acid, a promoter recognized by an RNA polymerase, and a nucleotide sequence complementary to a tag sequence indicative of the presence of said target nucleic acid in said sample;
providing a first sequestering agent which sepecifically interacts with said first complementary nucleic acid, wherein said first sequestering agent reduces the likelihood that said first complementary nucleic acid will hybridize to said non-target nucleic acids;
contacting said sample with said probe, such that said first complementary nucleic acid will hybridize to said first nucleotide sequence in said sample and said second complementary nucleic acid will hybridize to said second nucleotide sequence in said sample and said probe will adopt a circular conformation if said target nucleic acid is present in said sample;
ligating the ends of said circular probe together if said circular probe is present;
amplifying at least a portion of said probe comprising said promoter and said nucleotide sequence complementary to said tag;
performing a transcription reaction with said RNA polymerase, thereby generating a transcript comprising said tag if said sample contains said target nucleic acid; and
determining whether said transcript has been generated

96. The method of Paragraph 95, wherein said RNA polymerase is selected from the group consisting of T7 RNA polymerase, R4 RNA polymerase, T3 RNA polymerase, E. coli RNA polymerase, SP6 RNA polymerase, RNA polymerase II and RNA polymerase III.

97. The method of Paragraph 95, wherein said promoter is positioned 3′ of said first complementary nucleic acid and said nucleotide sequence complementary to said tag is positioned 5′ of said second complementary nucleic acid.

98. The method of Paragraph 95, further comprising performing an exonuclease digestion after ligating the ends of said probe together.

99. The method of Paragraph 95, further comprising providing a second sequestering agent which specifically interacts with said second complementary nucleic acid, wherein said second sequestering agent reduces the likelihood that said second complementary nucleic acid will hybridize to said non-target nucleic acids.

100. The method of Paragraph 95, wherein said first sequestering agent reduces the accessibility of the 5′ phosphate of said first complementary nucleic acid to a ligase when said first complementary nucleic acid is hybridized to said first sequestering agent.

101. The method of Paragraph 95, wherein said first sequestering agent reduces the accessibility of the 3′ hydroxyl of said first complementary nucleic acid to a ligase when said first complementary nucleic acid is hybridized to said sequestering agent.

102. The method of Paragraph 99, wherein said first sequestering agent reduces the accessibility of the 5′ phosphate of said first complementary nucleic acid to a ligase when said first complementary nucleic acid is hybridized to said first sequestering agent and said second sequestering agent reduces the accessibility of the 3′ hydroxyl of said second complementary nucleic acid to a ligase when said second complementary nucleic acid is hybridized to said second sequestering agent.

103. The method of Paragraph 95, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid.

104. The method of Paragraph 99 wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid and wherein said hybridizing step comprises hybridizing said second complementary nucleic acid to said second nucleotide sequence in said target nucleic acid at a temperature selected from the group consisting of a temperature at least 5 degrees Celsius below the melting temperature of the duplex between said second complementary nucleic acid and said second nucleotide sequence in said target nucleic acid, a temperature at least 10 degrees Celsius below the melting temperature of the duplex between said second complementary nucleic acid and said second nucleotide sequence in said target nucleic acid, a temperature at least 15 degrees Celsius below the melting temperature of the duplex between said second complementary nucleic acid and said second nucleotide sequence in said target nucleic acid, a temperature at least 20 degrees Celsius below the melting temperature of the duplex between said second complementary nucleic acid and said second nucleotide sequence in said target nucleic acid and a temperature at least 25 degrees Celsius below the melting temperature of the duplex between said second complementary nucleic acid and said second nucleotide sequence in said target nucleic acid.

105. A method for determining whether a target nucleic acid is present in a sample comprising:
providing a sample to be tested for the presence of a target nucleic acid said sample comprising non-target nucleic acids;
providing a probe comprising a first end comprising a first complementary nucleic acid complementary to a first nucleotide sequence in said target nucleic acid, a second end comprising a second complementary nucleic acid complementary to a second nucleotide sequence in said target nucleic acid, and a nucleotide sequence comprising a sequence complementary to or identical to a tag sequence indicative of the presence of said target nucleic acid;
providing a first sequestering agent which sepecifically interacts with said first complementary nucleic acid, wherein said first sequestering agent reduces the likelihood that said first complementary nucleic acid will hybridize to said non-target nucleic acids;
contacting said sample with said probe, such that said first complementary nucleic acid will hybridize to said first nucleotide sequence in said sample and said second complementary nucleic acid will hybridize to said second nucleotide sequence in said sample and said probe will adopt a circular conformation if said target nucleic acid is present in said sample;
ligating the ends of said circular probe together if said circular probe is present;
amplifying at least a portion of said probe comprising said tag, thereby generating an amplification product if said sample contains said target nucleic acid; and
determining whether said amplification product has been generated.

106. The method of Paragraph 105, wherein said nucleotide sequence comprising said tag is positioned 5′ of said second complementary nucleic acid.

107. The method of Paragraph 105, further comprising performing an exonuclease digestion after ligating the ends of said probe together.

108. The method of Paragraph 105, further comprising providing a second sequestering agent which specifically interacts with said second complementary nucleic acid, wherein said second sequestering agent reduces the likelihood that said second complementary nucleic acid will hybridize to said non-target nucleic acids.

109. The method of Paragraph 105, wherein said first sequestering agent reduces the accessibility of the 5′ phosphate of said first complementary nucleic acid to a ligase when said first complementary nucleic acid is hybridized to said first sequestering agent.

110. The method of Paragraph 105, wherein said first sequestering agent reduces the accessibility of the 3′ hydroxyl of said first complementary nucleic acid to a ligase when said first complementary nucleic acid is hybridized to said sequestering agent.

111. The method of Paragraph 108, wherein said first sequestering agent reduces the accessibility of the 5′ phosphate of said first complementary nucleic acid to a ligase when said first complementary nucleic acid is hybridized to said first sequestering agent and said second sequestering agent reduces the accessibility of the 3′ hydroxyl of said second complementary nucleic acid to a ligase when said second complementary nucleic acid is hybridized to said second sequestering agent.

112. The method of Paragraph 105, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid.

113. The method of Paragraph 108 wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid and wherein said hybridizing step comprises hybridizing said second complementary nucleic acid to said second nucleotide sequence in said target nucleic acid at a temperature selected from the group consisting of a temperature at least 5 degrees Celsius below the melting temperature of the duplex between said second complementary nucleic acid and said second nucleotide sequence in said target nucleic acid, a temperature at least 10 degrees Celsius below the melting temperature of the duplex between said second complementary nucleic acid and said second nucleotide sequence in said target nucleic acid, a temperature at least 15 degrees Celsius below the melting temperature of the duplex between said second complementary nucleic acid and said second nucleotide sequence in said target nucleic acid, a temperature at least 20 degrees Celsius below the melting temperature of the duplex between said second complementary nucleic acid and said second nucleotide sequence in said target nucleic acid and a temperature at least 25 degrees Celsius below the melting temperature of the duplex between said second complementary nucleic acid and said second nucleotide sequence in said target nucleic acid.

114. A method for determining whether a target nucleic acid is present in a sample comprising:
providing a sample to be tested for the presence of a target nucleic acid said sample comprising non-target nucleic acids;
performing an amplification reaction on said sample, thereby generating an amplified target nucleic acid if said sample contains said target nucleic acid;
providing a first complementary nucleic acid comprising a promoter recognized by an RNA polymerase positioned 3′ of a sequence complementary to a first nucleotide sequence in said target nucleic acid;
providing a second complementary nucleic acid comprising a sequence complementary to a tag sequence indicative of the presence of said target nucleic acid in said sample positioned 5′ of a sequence complementary to a second nucleotide sequence in said target nucleic acid, wherein said second complementary nucleic acid is on a separate molecule from said first complementary nucleic acid;
providing a first sequestering agent which sepecifically interacts with said first complementary nucleic acid, wherein said first sequestering agent reduces the likelihood that said first complementary nucleic acid will hybridize to said non-target nucleic acids;
contacting said sample with said first complementary nucleic acid and said second complementary nucleic acid, such that said first complementary nucleic acid will hybridize to said first nucleotide sequence in said amplified target nucleic acid and said second complementary nucleic acid will hybridize to said second nucleotide sequence in said amplified target nucleic acid;
ligating said first complementary nucleic acid to said second complementary nucleic acid if said target nucleic acid is present in said sample;
performing a transcription reaction with said RNA polymerase, thereby generating a transcript comprising said tag if said sample contains said target nucleic acid; and
determining whether said transcript has been generated.

115. The method of Paragraph 114, wherein said RNA polymerase is selected from the group consisting of T7 RNA polymerase, R4 RNA polymerase, T3 RNA polymerase, E. coli RNA polymerase, SP6 RNA polymerase, RNA polymerase II and RNA polymerase III.

116. The method of Paragraph 114, wherein said first complementary nucleic acid is resistant to digestion by a 3′ exonuclease and said second complementary nucleic acid is resistant to digestion by a 5′ exonduclease and wherein said method further comprises performing a digestion with a 3′ exonuclease and a 5′ exonuclease after ligating said first complementary nucleic acid to said second complementary nucleic acid if said sample contains said target nucleic acid.

117. The method of Paragraph 114, further comprising providing a second sequestering agent which specifically interacts with said second complementary nucleic acid, wherein said second sequestering agent reduces the likelihood that said second complementary nucleic acid will hybridize to said non-target nucleic acids.

118. The method of Paragraph 114, wherein said first sequestering agent reduces the accessibility of the 5′ phosphate of said first complementary nucleic acid to a ligase when said first complementary nucleic acid is hybridized to said first sequestering agent.

119. The method of Paragraph 117, wherein said first sequestering agent reduces the accessibility of the 5′ phosphate of said first complementary nucleic acid to a ligase when said first complementary nucleic acid is hybridized to said first sequestering agent and said second sequestering agent reduces the accessibility of the 3′ hydroxyl of said second complementary nucleic acid to a ligase when said second complementary nucleic acid is hybridized to said second sequestering agent.

120. The method of Paragraph 114, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid.

121. The method of Paragraph 117 wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid and wherein said hybridizing step comprises hybridizing said second complementary nucleic acid to said second nucleotide sequence in said target nucleic acid at a temperature selected from the group consisting of a temperature at least 5 degrees Celsius below the melting temperature of the duplex between said second complementary nucleic acid and said second nucleotide sequence in said target nucleic acid, a temperature at least 10 degrees Celsius below the melting temperature of the duplex between said second complementary nucleic acid and said second nucleotide sequence in said target nucleic acid, a temperature at least 15 degrees Celsius below the melting temperature of the duplex between said second complementary nucleic acid and said second nucleotide sequence in said target nucleic acid, a temperature at least 20 degrees Celsius below the melting temperature of the duplex between said second complementary nucleic acid and said second nucleotide sequence in said target nucleic acid and a temperature at least 25 degrees Celsius below the melting temperature of the duplex between said second complementary nucleic acid and said second nucleotide sequence in said target nucleic acid.

122. A method for determining whether a target nucleic acid is present in a sample comprising:
providing a sample to be tested for the presence of a target nucleic acid said sample comprising non-target nucleic acids;
performing an amplification reaction on said sample, thereby generating an amplified target nucleic acid if said sample contains said target nucleic acid;
providing a first complementary nucleic acid comprising a sequence complementary to a first nucleotide sequence in said target nucleic acid;
providing a second complementary nucleic acid comprising a tag sequence indicative of the presence of said target nucleic acid in said sample positioned 5′ of a sequence complementary to a second nucleotide sequence in said target nucleic acid, wherein said second complementary nucleic acid is on a separate molecule from said first complementary nucleic acid;
providing a first sequestering agent which sepecifically interacts with said first complementary nucleic acid, wherein said first sequestering agent reduces the likelihood that said first complementary nucleic acid will hybridize to said non-target nucleic acids;
contacting said sample with said first complementary nucleic acid and said second complementary nucleic acid, such that said first complementary nucleic acid will hybridize to said first nucleotide sequence in said amplified target nucleic acid and said second complementary nucleic acid will hybridize to said second nucleotide sequence in said amplified target nucleic acid p1 ligating said first complementary nucleic acid to said second complementary nucleic acid if said target nucleic acid is present in said sample, thereby generating a ligation product if said target nucleic acid is present in said sample; and
determining whether said ligation product has been generated.

123. The method of Paragraph 122, wherein said first complementary nucleic acid is resistant to digestion by a 3′ exonuclease and said second complementary nucleic acid is resistant to digestion by a 5′ exonduclease and wherein said method further comprises performing a digestion with a 3′ exonuclease and a 5′ exonuclease after ligating aid first complementary nucleic acid to said second complementary nucleic acid if said ample contains said target nucleic acid.

124. The method of Paragraph 122, further comprising providing a second equestering agent which specifically interacts with said second complementary nucleic acid, wherein said second sequestering agent reduces the likelihood that said second complementary nucleic acid will hybridize to said non-target nucleic acids.

125. The method of Paragraph 122, wherein said first sequestering agent reduces the accessibility of the 5′ phosphate of said first complementary nucleic acid to a ligase when said first complementary nucleic acid is hybridized to said first sequestering agent.

126. The method of Paragraph 124, wherein said first sequestering agent reduces the accessibility of the 5′ phosphate of said first complementary nucleic acid to a ligase when said first complementary nucleic acid is hybridized to said first sequestering agent and said second sequestering agent reduces the accessibility of the 3′ hydroxyl of said second complementary nucleic acid to a ligase when said second complementary nucleic acid is hybridized to said second sequestering agent.

127. The method of Paragraph 122, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid.

128. The method of Paragraph 124 wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid and wherein said hybridizing step comprises hybridizing said second complementary nucleic acid to said second nucleotide sequence in said target nucleic acid at a temperature selected from the group consisting of a temperature at least 5 degrees Celsius below the melting temperature of the duplex between said second complementary nucleic acid and said second nucleotide sequence in said target nucleic acid, a temperature at least 10 degrees Celsius below the melting temperature of the duplex between said second complementary nucleic acid and said second nucleotide sequence in said target nucleic acid, a temperature at least 15 degrees Celsius below the melting temperature of the duplex between said second complementary nucleic acid and said second nucleotide sequence in said target nucleic acid, a temperature at least 20 degrees Celsius below the melting temperature of the duplex between said second complementary nucleic acid and said second nucleotide sequence in said target nucleic acid and a temperature at least 25 degrees Celsius below the melting temperature of the duplex between said second complementary nucleic acid and said second nucleotide sequence in said target nucleic acid.

129. A method for determining whether a target nucleic acid is present in a sample comprising:
providing a sample to be tested for the presence of a target nucleic acid said sample comprising non-target nucleic acids;
providing a first complementary nucleic acid comprising a promoter recognized by an RNA polymerase positioned 3′ of a sequence complementary to a first nucleotide sequence in said target nucleic acid;
providing a second complementary nucleic acid comprising a sequence complementary to a tag sequence indicative of the presence of said target nucleic acid in said sample positioned 5′ of a sequence complementary to a second nucleotide sequence in said target nucleic acid, wherein said second complementary nucleic acid is on a separate molecule from said first complementary nucleic acid;
providing a first sequestering agent which sepecifically interacts with said first complementary nucleic acid, wherein said first sequestering agent reduces the likelihood that said first complementary nucleic acid will hybridize to said non-target nucleic acids;
contacting said sample with said first complementary nucleic acid and said second complementary nucleic acid, such that said first complementary nucleic acid will hybridize to said first nucleotide sequence in said target nucleic acid and said second complementary nucleic acid will hybridize to said second nucleotide sequence in said target nucleic acid
ligating said first complementary nucleic acid to said second complementary nucleic acid if said target nucleic acid is present in said sample, thereby generating a ligation product if said target nucleic acid is present in said sample;
amplifying said ligation product if present;
performing a transcription reaction on said amplified ligation product with said RNA polymerase, thereby generating a transcript comprising said tag if said sample contains said target nucleic acid; and
determining whether said transcript has been generated.

130. The method of Paragraph 129, wherein said RNA polymerase is selected from the group consisting of T7 RNA polymerase, R4 RNA polymerase, T3 RNA polymerase, E. coli RNA polymerase, SP6 RNA polymerase, RNA polymerase II and RNA polymerase III.

131. The method of Paragraph 129, wherein said first complementary nucleic acid is resistant to digestion by a 3′ exonuclease and said second complementary nucleic acid is resistant to digestion by a 5′ exonduclease and wherein said method further comprises performing a digestion with a 3′ exonuclease and a 5′ exonuclease after ligating said first complementary nucleic acid to said second complementary nucleic acid if said sample contains said target nucleic acids.

132. The method of Paragraph 129, further comprising providing a second sequestering agent which specifically interacts with said second complementary nucleic acid, wherein said second sequestering agent reduces the likelihood that said second complementary nucleic acid will hybridize to said non-target nucleic acids.

133. The method of Paragraph 129, wherein said first sequestering agent reduces the accessibility of the 5′ phosphate of said first complementary nucleic acid to a ligase when said first complementary nucleic acid is hybridized to said first sequestering agent.

134. The method of Paragraph 132, wherein said first sequestering agent reduces the accessibility of the 5′ phosphate of said first complementary nucleic acid to a ligase when said first complementary nucleic acid is hybridized to said first sequestering agent and said second sequestering agent reduces the accessibility of the 3′ hydroxyl of said second complementary nucleic acid to a ligase when said second complementary nucleic acid is hybridized to said second sequestering agent.

135. The method of Paragraph 129, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid.

136. The method of Paragraph 132 wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid and wherein said hybridizing step comprises hybridizing said second complementary nucleic acid to said second nucleotide sequence in said target nucleic acid at a temperature selected from the group consisting of a temperature at least 5 degrees Celsius below the melting temperature of the duplex between said second complementary nucleic acid and said second nucleotide sequence in said target nucleic acid, a temperature at least 10 degrees Celsius below the melting temperature of the duplex between said second complementary nucleic acid and said second nucleotide sequence in said target nucleic acid, a temperature at least 15 degrees Celsius below the melting temperature of the duplex between said second complementary nucleic acid and said second nucleotide sequence in said target nucleic acid, a temperature at least 20 degrees Celsius below the melting temperature of the duplex between said second complementary nucleic acid and said second nucleotide sequence in said target nucleic acid and a temperature at least 25 degrees Celsius below the melting temperature of the duplex between said second complementary nucleic acid and said second nucleotide sequence in said target nucleic acid.

137. A method for determining whether a target nucleic acid is present in a sample comprising:
providing a sample to be tested for the presence of a target nucleic acid said sample comprising non-target nucleic acids;
providing a first complementary nucleic acid comprising a sequence complementary to a first nucleotide sequence in said target nucleic acid;
providing a second complementary nucleic acid comprising a sequence complementary to or identical to a tag indicative of the presence of said target nucleic acid in said sample positioned 5′ of a sequence complementary to a second nucleotide sequence in said target nucleic acid, wherein said second complementary nucleic acid is on a separate molecule from said first complementary nucleic acid;
providing a first sequestering agent which sepecifically interacts with said first complementary nucleic acid, wherein said first sequestering agent reduces the likelihood that said first complementary nucleic acid will hybridize to said non-target nucleic acids;
contacting said sample with said first complementary nucleic acid and said second complementary nucleic acid, such that said first complementary nucleic acid will hybridize to said first nucleotide sequence in said target nucleic acid and said second complementary nucleic acid will hybridize to said said second nucleotide sequence in said target nucleic acid
ligating said first complementary nucleic acid to said second complementary nucleic acid if said target nucleic acid is present in said sample, thereby generating a ligation product if said target nucleic acid is present in said sample;
amplifying said ligation product if present; and
determining whether said ligation product has been generated.

138. The method of Paragraph 137, wherein said first complementary nucleic acid is resistant to digestion by a 3′ exonuclease and said second complementary nucleic acid is resistant to digestion by a 5′ exonduclease and wherein said method further comprises performing a digestion with a 3′ exonuclease and a 5′ exonuclease after ligating said first complementary nucleic acid to said second complementary nucleic acid if said sample contains said target nucleic acid.

139. The method of Paragraph 137, further comprising providing a second sequestering agent which specifically interacts with said second complementary nucleic acid, wherein said second sequestering agent reduces the likelihood that said second complementary nucleic acid will hybridize to said non-target nucleic acids.

140. The method of Paragraph 137, wherein said first sequestering agent reduces the accessibility of the 5′ phosphate of said first complementary nucleic acid to a ligase when said first complementary nucleic acid is hybridized to said first sequestering agent.

141. The method of Paragraph 139, wherein said first sequestering agent reduces the accessibility of the 5′ phosphate of said first complementary nucleic acid to a ligase when said first complementary nucleic acid is hybridized to said first sequestering agent and said second sequestering agent reduces the accessibility of the 3′ hydroxyl of said second complementary nucleic acid to a ligase when said second complementary nucleic acid is hybridized to said second sequestering agent.

142. The method of Paragraph 137, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid.

143. The method of Paragraph 139 wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid and wherein said hybridizing step comprises hybridizing said second complementary nucleic acid to said second nucleotide sequence in said target nucleic acid at a temperature selected from the group consisting of a temperature at least 5 degrees Celsius below the melting temperature of the duplex between said second complementary nucleic acid and said second nucleotide sequence in said target nucleic acid, a temperature at least 10 degrees Celsius below the melting temperature of the duplex between said second complementary nucleic acid and said second nucleotide sequence in said target nucleic acid, a temperature at least 15 degrees Celsius below the melting temperature of the duplex between said second complementary nucleic acid and said second nucleotide sequence in said target nucleic acid, a temperature at least 20 degrees Celsius below the melting temperature of the duplex between said second complementary nucleic acid and said second nucleotide sequence in said target nucleic acid and a temperature at least 25 degrees Celsius below the melting temperature of the duplex between said second complementary nucleic acid and said second nucleotide sequence in said target nucleic acid.

144. A method for determining whether a target nucleic acid is present in a sample comprising:
providing a sample to be tested for the presence of a target nucleic acid said sample comprising non-target nucleic acids;
providing a probe comprising a first end comprising a first complementary nucleic acid complementary to a first nucleotide sequence in said target nucleic acid and a second end comprising a second complementary nucleic acid complementary to a second nucleotide sequence in said target nucleic acid;
providing a first sequestering agent which sepecifically interacts with said first complementary nucleic acid, wherein said first sequestering agent reduces the likelihood that said first complementary nucleic acid will hybridize to said non-target nucleic acids;
contacting said sample with said probe, such that said first complementary nucleic acid will hybridize to said first nucleotide sequence in said target nucleic acid and said second complementary nucleic acid will hybridize to said second nucleotide sequence in said target nucleic acid and said probe will adopt a circular conformation if said target nucleic acid is present in said sample;
ligating the ends of said circular probe together if said circular probe is present;
performing a rolling circle amplification procedure using said ciruclarized and ligated probe as a template, thereby generating an amplfication product if said sample contains said target nucleic acid; and
determining whether said amplification product has been generated.

145. The method of Paragraph 144, wherein said probe further comprises a sequence complementary to a tag indicative of the presence of said target nucleic acid, and said determing step comprises determining whether an amplfication product comprising said tag has been generated.

146. The method of Paragraph 144, further comprising performing an exonuclease digestion after ligating the ends of said probe together.

147. The method of Paragraph 144, further comprising providing a second sequestering agent which specifically interacts with said second complementary nucleic acid, wherein said second sequestering agent reduces the likelihood that said second complementary nucleic acid will hybridize to said non-target nucleic acids.

148. The method of Paragraph 144, wherein said first sequestering agent reduces the accessibility of the 5′ phosphate of said first complementary nucleic acid to a ligase when said first complementary nucleic acid is hybridized to said first sequestering agent.

149. The method of Paragraph 144, wherein said first sequestering agent reduces the accessibility of the 3′ hydroxyl of said first complementary nucleic acid to a ligase when said first complementary nucleic acid is hybridized to said sequestering agent.

150. The method of Paragraph 147, wherein said first sequestering agent reduces the accessibility of the 5′ phosphate of said first complementary nucleic acid to a ligase when said first complementary nucleic acid is hybridized to said first sequestering agent and said second sequestering agent reduces the accessibility of the 3′ hydroxyl of said second complementary nucleic acid to a ligase when said second complementary nucleic acid is hybridized to said second sequestering agent.

151. The method of Paragraph 144, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid.

152. The method of Paragraph 147 wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid and wherein said hybridizing step comprises hybridizing said second complementary nucleic acid to said second nucleotide sequence in said target nucleic acid at a temperature selected from the group consisting of a temperature at least 5 degrees Celsius below the melting temperature of the duplex between said second complementary nucleic acid and said second nucleotide sequence in said target nucleic acid, a temperature at least 10 degrees Celsius below the melting temperature of the duplex between said second complementary nucleic acid and said second nucleotide sequence in said target nucleic acid, a temperature at least 15 degrees Celsius below the melting temperature of the duplex between said second complementary nucleic acid and said second nucleotide sequence in said target nucleic acid, a temperature at least 20 degrees Celsius below the melting temperature of the duplex between said second complementary nucleic acid and said second nucleotide sequence in said target nucleic acid and a temperature at least 25 degrees Celsius below the melting temperature of the duplex between said second complementary nucleic acid and said second nucleotide sequence in said target nucleic acid.

The present disclosure provides methods and compositions for detecting targets in a sample using a universal tag assay. The universal tag assay disclosed and claimed herein provides tags and probes and universal detectors for use in a universal tag assay that advantageously minimizes spurious signals without the need to employ special conditions or special reagents. Targets are detected using the universal tag assay of the present invention by generating tagged molecules having identifier tags corresponding to targets, incubating tagged molecules with a universal detector having detection probes, and measuring hybridization of identifier tags to complementary detection probes, where hybridization of an identifier tag to its complementary detection probe indicates the presence of the target corresponding to that identifier tag.

The universal tag assay disclosed herein includes but is not limited to: a) a set of minimally cross-hybridizing tags and probes selected such that at least one tag will serve as an identifier tag for each target being assayed and each tag has a complementary detection probe in the universal detector; b) tagged molecules generated from the sample being assayed, where a tagged molecule containing an identifier tag for a target is only generated when that target is present in the sample; and c) a universal detector having detection probes coupled to a detection means in a manner such that hybridization of tags to complementary detection probes on the universal detector can be detected. The universal tag assay disclosed herein provides that detecting hybridization of an identifier tag to its complementary detection probe indicates the presence of the corresponding target in the sample being assayed. A tagged molecule may be a tagged target that contains a copy or complement of a target and the identifier tag for that target. Alternately, a tagged molecule may contain an identifier tag molecule for a target and no copy or complement of the target. Tagged molecules may be generated using target-dependent amplification methods, or may be generated by target-dependent methods that do not employ amplification of target, or by a combination of methods.

In accordance with certain methods described herein, the presence of a target nucleotide sequence in a sample may be detected by generating at least one tagged molecule in response to the target nucleotide sequence in a target-dependent manner, incubating tagged molecules with a universal detector having at least one detection probe, and measuring hybridization of identifier tags to complementary detection probes. A tagged molecule may be a tagged target that contains a copy or complement of the target nucleotide sequence and an identifier tag for that target nucleotide sequence. Alternately, a tagged molecule contains an identifier tag for a particular target sequence and no copy or complement of the target nucleotide sequence. Tagged molecules are incubated with a universal detector having detection probes coupled to a detection means, and hybridization of an identifier tag to a complementary detection probe on the universal detector generates a signal that indicates the presence of the corresponding target nucleotide sequence in the sample.

The universal detector includes detection probes attached to a surface, film, or particle, wherein detection probes are coupled to a detection means such that hybridization of a tag to a complementary probe can be detected. Detection probes may be immobilized in a fixed array, or may be attached to a surface, film, or particle in a manner that permits changing the location of the detection probes. One or more detection probes may be coupled to a particle such as a bead. Detection probes may be attached to a surface, film, or particle by covalent bonds, ionic bonds, electrostatic interactions or adsorptive interactions, and the attachment may be reversible. Alternately detection probe may be coupled to a detection means in solution, such that hybridization of a tag to a complementary probe can be detected.

Preferably, the universal tag assay of the present invention provides a universal chip that includes detection probes immobilized to a support including a detection means, such that hybridization of tags to complementary detection probes can be measured. Detection means for measuring hybridization of tags to complementary detection probes can be electrochemical, fluorescent, calorimetric, radiometric or magnetic. In particular, the universal tag assay of the present invention provides an array of detection probes coupled to electrodes attached to a substrate, and hybridization of oligonucleotide tags to oligonucleotide detection probes is detected by electrochemical methods. More preferably, gold or carbon electrodes are used to detect tag binding to detection probes. Even more preferably, hybridization of identifier tags to detection probes immobilized to gold or carbon electrodes is detected by ruthenium amperometry. The electrodes may be coated with avidin which can be bound by biotin-labeled oligonucleotides. Alternately, electrodes may be coated with another protein which can be bound by oligonucleotides derivatized with a moiety that binds the protein coating the electrode.

One aspect of the invention provides a method for detecting a target nucleotide sequence in a sample, where the method may include but is not limited to the following steps: a) obtaining template containing the target nucleotide sequence; b) amplifying the template to generate at least one tagged molecule having at least one copy or complement of the target nucleotide sequence and at least one tag sequence chosen as an identifier tag for the target nucleotide sequence; c) incubating at least one tagged molecule with a universal detector having detection probes coupled to a detection means; d) detecting hybridization of identifier tags to complementary detection probes on a universal detector. In accordance with the universal tag assay disclosed herein, detecting hybridization of an identifier tag to a complementary detection probe on a universal detector indicates the presence of the corresponding target nucleotide sequence in the sample being assayed. Another aspect provides a method for detecting multiple target nucleotide sequences in a sample, wherein each target nucleotide sequence has a distinct identifier tag.

One aspect of the invention provides a method for detecting a target nucleotide sequence in a sample, where the method may include but is not limited to the following steps: a) obtaining template containing the target nucleotide sequence; b) amplifying the template to generate at least one tagged molecule having at least one copy or complement of the target nucleotide sequence and at least one tag sequence chosen as an identifier tag for the target nucleotide sequence, as well as optional additional exogenous nucleotide sequences including sequences involved in trimming amplification products; c) trimming amplification products to generate at least one tagged molecule containing tag sequence chosen as an identifier tag for the target nucleotide sequence; d) incubating at least one tagged molecule with a universal detector capable of finding tag sequence; and e) detecting hybridization of identifier tags to complementary detection probes on a universal detector. In accordance with the universal tag assay disclosed herein, detecting hybridization of an identifier tag to a complementary detection probe on a universal detector indicates the presence of the corresponding target nucleotide sequence in the sample being assayed. Another aspect provides a method for detecting multiple target nucleotide sequences in a sample, wherein each target nucleotide sequence has a distinct identifier tag.

Another aspect of the invention provides a method for detecting a target nucleotide sequence in a sample, where the method may include but is not limited to the following steps: a) obtaining template containing the target nucleotide sequence; b) amplifying the template to generate at least one tagged molecule having at least one tag sequence chosen as an identifier tag for the target nucleotide sequence and optionally, at least one copy or complement of the target nucleotide sequence, as well as additional exogenous nucleotide sequences including sequences involved in trimming amplification products; c) trimming amplification products to generate at least one tagged molecule containing the identifier tag for the target nucleotide sequence and no copy or complement of the target nucleotide sequence; d) incubating at least one tagged molecule with a universal detector having a set of detection probes coupled to a detection means; e) detecting hybridization of identifier tags to complementary detection probes on a universal detector. In accordance with the universal tag assay disclosed herein, detecting hybridization of an identifier tag to a complementary detection probe on a universal detector indicates the presence of the corresponding target nucleotide sequence in the sample. Another aspect provides a method for detecting multiple target nucleotide sequences in a sample, wherein each target nucleotide sequence has a distinct identifier tag.

Yet another object of the present invention provides a set of complementary tags and probes suitable for use with the universal tag assay disclosed herein, preferably a set of minimally cross-hybridizing oligonucleotides wherein all tag-probe duplexes have the same or similar melting temperature and stacking energy. Another object provides a set of probes and a set of tags such that each tag in the set has a complementary detection probe coupled to a detection means in the universal detector, with the result that not only does hybridization of a tag to its complementary detection probe reliably indicate the presence of the corresponding target in a sample, but also the absence of hybridization of a tag to its complementary detection probe reliably indicates the absence of the corresponding target in a sample. Preferably, the reliability of universal tag assay is increased by including tags and probes that serve as internal controls for reagent quality, hybridization conditions, and other parameters.

In accordance with one aspect of the methods for detecting target nucleotide sequences disclosed herein, rolling circle (RC) amplification of a suitable template may be used for the amplification step. In embodiments using RC amplification, the RC probe used to amplify template containing a target nucleotide sequence includes a portion of sequence complementary to the target nucleotide sequence and further includes sequence complementary to an identifier tag for that target nucleotide sequence, such that the amplification products contain a copy of the target nucleotide sequence and a distinct identifier tag sequence capable of hybridizing to a detection probe. Preferably, products of RC amplification contain additional exogenous sequences not found in the target nucleotide sequence or tag sequence, which may include but are not limited to sequences involved in trimming amplification products, sequences involved in primer binding, or sequences involved in forming polymerase promoters. Alternately, RC amplification may be carried out on copies or complements of nucleotide sequence. Aspects of the present invention provide that RC amplification can be carried out in linear mode or non-linear mode. In one preferred embodiment, RC amplification in linear mode generates single-stranded amplification products. In another preferred embodiment, RC amplification in non-linear, or exponential mode using at least one additional primer complementary to a portion of the amplification product generates double-stranded amplification products, preferably hyperbranched amplification products. The template for RC amplification may be DNA or RNA, including but not limited to genomic DNA, cDNA, PCR products, ligation products including LCR products, RC amplification products, synthetic DNA, mRNA, rRNA, RC transcription products, or synthetic RNA. Single-stranded template may be obtained by denaturing double-stranded DNA, preferably to generate single-stranded template from the target strand containing at least one target nucleotide sequence. The double-stranded DNA may be genomic DNA, cDNA, PCR products, or ligation products including LCR products.

In accordance with another aspect of the invention, RC amplification is used to amplify PCR products or LCR products containing a copy or complement of the target nucleotide sequence. In one embodiment, a PCR product containing a complement of the target nucleotide sequence is amplified using an RC probe having a copy of the target nucleotide sequence and sequence complementary to an identifier tag for that target nucleotide sequence, such that the amplification products contain at least one complement of the target nucleotide sequence and an identifier tag capable of hybridizing to a detection probe. Optionally, the RC amplification products are trimmed.

In accordance with another aspect of the present invention, more than one amplification of target nucleotide sequence is carried out. Ligase chain reaction (LCR), non-enzymatic ligation, or PCR can be used to amplify target nucleotide sequence. LCR products, non-enzymatic ligation products, and PCR products may be amplified in a subsequent amplification step, preferably an RC amplification step.

LCR or non-enzymatic ligation amplification of target nucleotide sequence includes but is not limited to the following steps: a) if necessary, obtaining single-stranded template having at least one target nucleotide sequence; b) contacting the template with a plurality of oligonucleotide ligation primers, where at least one pair of the ligation primers is designed to hybridize to at least one target nucleotide sequence on the template, such that the 5′ end of one of the pair of ligation primers hybridizes adjacent to the 3′ end of the other of the pair of ligation primers; c) incubating template and ligation primers under conditions that promote adjacent hybridization of at least one pair of ligation primers to the target nucleotide sequence on the template and ligation of any adjacent hybridized pair of ligation primers to form at least one ligation product that includes sequence complementary to the target nucleotide sequence; d) dissociating the ligation product from the template; e) repeating the hybridization and ligation steps as desired; f) recovering the ligation products for use in subsequent amplification steps. In one embodiment, ligation reactions, preferably LCR, are repeated using temperature cycling for exponential amplification of the target nucleotide sequence.

In a preferred embodiment, each ligation primer including sequence complementary to the target nucleotide sequence on the target strand also includes exogenous nucleotide sequence complementary to a portion of the “backbone” of a circularizable RC padlock probe in linear form that contains a copy of the target nucleotide sequence and a complement of an identifier tag sequence. In such an embodiment, the RC padlock probe in linear form has 3′ sequence corresponding to a region of target nucleotide sequence, and 5′ sequence corresponding to a region of target nucleotide sequence, where the 3′ and 5′ sequence is separated by a “backbone” region that does not contain sequence corresponding to target nucleotide sequence. The ligation product includes 5′ and 3′ exogenous nucleotide sequence complementary to a portion of the backbone of the RC padlock probe, where the sequence complementary to a portion of the backbone of the RC padlock probe flanks sequence complementary to the target nucleotide sequence. The ligation product is then incubated with at least one RC padlock probe in linear form under conditions that promote hybridization of the RC padlock probe to the ligation product, such that the 5′ end of the RC padlock probe is adjacent to the 3′ end of the RC padlock probe and the 5′ and 3′ ends are ligated to form a circularized RC padlock probe. DNA polymerase is added to the complex formed by the RC padlock probe and the ligation product, under conditions that permit RC amplification of the RC padlock probe using the ligation product as a polymerization primer. In this embodiment, the amplification product is a single-stranded DNA molecule containing multiple copies of the RC probe sequence, including sequence complementary to the target nucleotide sequence and identifier tag sequence corresponding to the target nucleotide sequence. This amplification product may be used as a tagged molecule in the universal tag assay. Optionally, the amplification product may contain additional exogenous nucleotide sequence. The amplification product may include modified nucleotides, addressable ligands, or other modifications. In another embodiment, the amplification product includes at least one additional exogenous nucleotide sequence involved in post-amplification trimming of the amplification product to yield smaller tagged molecules for use in the universal tag assay. The amplification product may also contain primer binding sites for additional amplification steps, for example to generate double-stranded amplification products. The amplification product may further include sequences involved in forming promoter regions, preferably for polymerases.

In accordance with another aspect of the invention, additional exogenous nucleotide sequences not found in the target or the identifier tag may be introduced during an amplification step, wherein such exogenous nucleotide sequences may include sequences involved in trimming amplification products. In one embodiment, the exogenous nucleotide sequence may contain self-complementary sequences that form hairpin structures. These self-complementary sequences that form hairpin structures may contain at least one restriction enzyme recognition site for a restriction enzyme involved in the trimming step, and suitable restriction enzymes include Type II restriction enzymes such as EcoRI, or Type IIS restriction enzymes such as FokI. In another embodiment, exogenous nucleotide sequences introduced during an amplification step encode one strand of the restriction enzyme recognition site, and a double-stranded restriction enzyme recognition site is formed upon addition of at least one auxiliary oligonucleotide. Suitable restriction enzymes include Type II restriction enzymes such as EcoRI, or Type IIS restriction enzymes such as FokI.

In accordance with another aspect of the invention, additional exogenous nucleotide sequence not found in the target nucleotide sequence of the tag sequence may be introduced during an amplification step, wherein such exogenous sequences may include sequences involved in forming promoter regions for binding of polymerases to amplification products. In a preferred embodiment, double-stranded amplification product includes exogenous nucleotide sequence encoding a promoter for DNA or RNA polymerase, preferably T7 RNA polymerase, T7 DNA polymerase, Bst DNA polymerase, or phi 29 (φ29) DNA polymerase.

In accordance with one aspect of the present invention, ligation reactions are used to identify variant or polymorphic sequences of the target nucleotide sequence present in a sample. The variant sequence may be a single nucleotide polymorphism (SNP). Alternately, the variant sequence represents mutant or allelic forms, or splice variants, of a target nucleotide sequence. In a preferred embodiment, the amplification step is carried out using a plurality of RC padlock probes in linear form having sequences complementary to variant sequences of the target nucleotide sequence, wherein each RC probe in linear form is complementary to a single variant sequence and each probe includes complement of the identifier tag for that variant sequence. A sample is incubated with this plurality of RC padlock probes in linear form under conditions suitable for hybridization and ligation of RC padlock probes, such that only those RC padlock probes complementary to the variant sequence present in the sample will hybridize to the variant sequence and be ligated to form a circularized RC padlock probe suitable to generate tagged molecules for use with the universal tag assay. Hybridization of an identifier tag to a detector probe indicates which variant sequence was present, because only the RC probe complementary to the variant sequence present in the sample was circularized and amplified to generate the identifier tag capable of binding to a detection probe.

In accordance with another aspect, a plurality of variant sequences of the same or different target nucleotide sequences can be detected in a single reaction using a plurality of RC padlock probes in linear form as described above, wherein each RC padlock probe in linear form includes sequence complementary to a single variant sequence and complement of the identifier tag for that variant sequence. Hybridization of identifier tags to complementary detection probes indicates which variant sequences are present in the sample being assayed, because only those RC probes complementary to the variant sequences present in the sample were circularized and amplified to generate tagged molecules containing identifier tags capable of binding to complementary detection probes.

Alternately, ligation of primers can be used to identify variant or polymorphic sequences present in a sample. Ligation may be carried out using LCR or non-enzymatic ligation. A sample is incubated with ligation primers having sequences complementary to variant sequences of the target nucleotide sequence under conditions suitable for hybridization and ligation, wherein only those ligation primers complementary to the variant sequence present in the target strand template will hybridize to the template and form at least one ligation product having sequence that is complementary to the variant target nucleotide sequence present in the template. In another preferred embodiment, the set of ligation primers includes primers having exogenous sequence such that any ligation product includes exogenous nucleotide sequence flanking (3′ and 5′) sequence complementary to a portion of the variant target nucleotide sequence. A plurality of variant sequences of the same or different target nucleotide sequences may be detected in a single reaction using a plurality of ligation primers as described above, wherein only those ligation primers having sequence complementary to a variant sequence will produce a ligation product complementary to that variant sequence. Ligation products having sequence complementary to variant sequences may be amplified to generate tagged molecules suitable for use with the universal tag assay disclosed herein. Optionally, ligation reactions can be carried out on PCR products containing copies or complements of target nucleotide sequence.

In another preferred embodiment, ligation primers complementary to variant sequences further contain exogenous sequence. In one embodiment, the 5′ end of the primer complementary to the region of target sequence 3′ (“downstream”) to the point of variant sequence contains identifier tag sequence, and the 3′ end of the primer complementary to the region of target sequence 5′ (“upstream”) to the point of variant sequence contains an RNA polymerase promoter sequence. The two primers are called the tag sequence primer and the promoter sequence primer, respectively. A ligation product is formed by ligation of a pair of primers complementary to the variant sequence present in the sample being assayed. The ligation product has an identifier tag, sequence complementary to the variant sequence, and one strand of an RNA polymerase promoter. Upon addition of an auxiliary oligonucleotide complementary to the promoter sequence, RNA transcription can be initiated. Transcription of the tag sequence occurs only if ligation resulted in joining the two halves of the target sequence. The target can be genomic DNA, RNA, or a copy of the target amplified by methods such as PCR, LCR, or RC amplification. In a preferred embodiment, the strand complementary to target is removed, for example by biotin or hybridization capture or by selective exonuclease digestion, to enhance the efficiency of ligation of the promoter ligation primer and tag ligation primer. A plurality of variant sequences of the same or different target nucleotide sequences may be detected in a single reaction using a plurality of ligation primers as described above, wherein only those ligation primers having sequence complementary to a variant sequence will generate a ligation product complementary to that variant sequence. Transcription of ligation products generates tagged RNA molecules containing identifier tags. Hybridization of RNA identifier tags to complementary detection probes indicates which variant sequences are present in the sample being assayed. In an alternative embodiment, the promoter-tag ligation can result from an LCR amplification, wherein the LCR primer complementary to the target sequence in the tag ligation primer does not contain the complement of the tag sequence.

Another aspect of the present invention is directed to methods for identifying an organism or individual by detecting a target nucleotide sequence chosen to serve as a distinguishing feature. An organism or individual is identified using some or all of the following steps: a) obtaining a sample from the organism or individual, where the sample contains template having at least one target nucleotide sequence; b) generating tagged molecules in a target-dependent maimer; c) optionally, trimming tagged molecules to generate smaller tagged molecules; d) incubating tagged molecules with a universal detector having an array of detection probes coupled to a detection means; and e) detecting hybridization of identifier tags to complementary detection probes. In one embodiment, an organism or individual is identified by hybridization of an identifier tag to its complementary detection probe, where the tagged molecules containing an identifier tag corresponding the target were generated because the target was present in the sample being assayed. In another embodiment, an organism or individual may be identified not only by hybridization of an identifier tag to its complementary detection probe, which reliably indicates the presence of the corresponding target in the sample being assayed, but also by the absence of hybridization of an identifier tag to its complementary detection probe, which reliably indicates the absence of the corresponding target in the sample being assayed. Preferably, at least one internal control is included in each assay in order to increase the reliability of results based on hybridization or lack of hybridization. More preferably, a plurality of internal controls are included. A plurality of targets in an individual or organism may be assayed using the universal tag assay. A plurality of individuals or organisms may be identified using the universal tag assay.

### BRIEF DESCRIPTION OF THE DRAWINGS

FIG. 1. Rolling Circle Amplification of Ligation Product. FIG. 1 illustrates one embodiment of the present invention in which a rolling circle probe comprising a first complementary nucleic acid complementary to a first nucleotide sequence in the target nucleic acid and a second complementary nucleic acid complementary to a second nucleotide sequence in the target nucleic acid is provided along with first and second sequestering agents comprising nucleotide sequences complementary to a portion of the first and second complementary nucleic acids respectively. The rolling circle probe comprises a sequence complementary to a tag to be used to indicate the presence of the target nucleotide sequence in a sample. Duplexes between the first and second complementary nucleic acids and the target nucleotide sequence are in equilibrium with duplexes between the first and second complementary nucleic acids and the first and second sequestering agents. Duplexes between the first and second complementary nucleic acids and the target nucleotide sequence predominate since they are thermodynamically favored over duplexes between the first and second complementary nucleic acids and the first and second sequestering agents. In addition, in the depicted embodiment, the sequestering agents block access to the reactive groups for ligation on the 3′ and 5′ ends of the complementary nucleic acids, thereby reducing non-templated ligation (ligation of complementary nucleic acids which are not hybridized to the target nucleic acid). The first and second complementary nucleic acids which are hybridized to the target nucleotide sequence are ligated together to form a circular molecule. Rolling circle amplification is performed on the circular molecule to generate a product comprising repeating units of the first and second complementary nucleic acids, the probe, and the sequence complementary to the tag. The rolling circle amplification product is trimmed to generate molecules comprising the sequence complementary to the tag which are hybridized to a universal chip to indicate the presence of the target nucleotide sequence in the sample.

FIG. 2. Allele-specific ligation of primers on a single-stranded target sequence that can be produced by methods such as PCR, LCR, or linear RC amplification. One primer (promoter primer) has a target-specific region connected to a promoter sequence; the other (tag primer) has a target-specific region connected to an identifier tag sequence. First and second sequestering agents comprising nucleotide sequences complementary to portions of the promoter primer and the tag primer respectively are provided. Duplexes between the first and second complementary nucleic acids and the target nucleotide sequence are in equilibrium with duplexes between the promoter primer and the tag primer and the first and second sequestering agents respectively. Duplexes between the promoter primer and the tag primer and the target nucleotide sequence predominate since they are thermodynamically favored over duplexes between the promoter primer and the tag primer and the first and second sequestering agents respectively. In addition, in the depicted embodiment, the sequestering agents block access to the reactive groups for ligation on the 3′ and 5′ ends of the complementary nucleic acids, thereby reducing non-templated ligation (ligation of complementary nucleic acids which are not hybridized to the target nucleic acid). Allele-specific ligation joins the tag primer and promoter primer which are hybridized to the target nucleotide sequence to form a ligation product having tag sequence and promoter sequence. A promoter oligonucleotide is provided which hybridizes to the promoter sequence in the ligation product to provide a double-stranded site on which to initiate transcription. Transcription of the ligation product produces several copies of the identifier tag sequence. Products of transcription are exposed to a universal chip for hybridization of the identifier tag to a complementary detection probe on the chip. The hybridization of tag sequence to the chip is detected.

FIG. 3. FIG. 3 illustrates one embodiment of a method for determining which allele(s) of a SNP are present in a sample. Three complementary nucleic acids, two of which are specific to one of the two alleles of the SNP and one of which hybridizes adjacent to the polymorphic nucleotide are provided. Sequestering agents which interact with each of the complementary nucleic acids are also provided. Duplexes between the complementary nucleic acids and the target nucleotide sequence are in equilibrium with duplexes between the complementary nucleic acids and the corresponding sequestering agent. Duplexes between the complementary nucleic acid and the target nucleotide sequence predominate since they are thermodynamically favored over duplexes between the complementary nucleic acids and the corresponding sequestering agent. In addition, in the depicted embodiment, the sequestering agents block access to the reactive groups for ligation on the 3′ and 5′ ends of the complementary nucleic acids, thereby reducing non-templated ligation (ligation of complementary nucleic acids which are not hybridized to the target nucleic acid). If the sample contains a given allele of the SNP, a ligation product comprising the complementary nucleic acid specific for that allele and the complementary nucleic acid which hybridizes adjacent to the polymorphic nucleotide will be formed from the complementary nucleic acids which are hybridized to the target nucleotide sequence. The ligation product is amplified using PCR and detected.

FIG. 4. FIG. 4 illustrates one embodiment of a method in which both the first complementary nucleic acid and the second complementary nucleic acid are on a single molecule which also includes a T7 promoter and a nucleotide sequence complementary to a tag to be used to indicate the presence of a target sequence in the sample. Sequestering agents which interact with the first and second complementary nucleic acids are provided. Duplexes between the first and second complementary nucleic acids and the target nucleotide sequence are in equilibrium with duplexes between the first and second complementary nucleic acids and the first and second sequestering agents respectively. Duplexes between the first and second complementary nucleic acids and the target nucleotide sequence predominate since they are thermodynamically favored over duplexes between the first and second complementary nucleic acids and the first and second sequestering agents respectively. In addition, in the depicted embodiment, the sequestering agents block access to the reactive groups for ligation on the 3′ and 5′ ends of the complementary nucleic acids, thereby reducing non-templated ligation (ligation of complementary nucleic acids which are not hybridized to the target nucleic acid). If the sample contains the target nucleic acid, a circular ligation product is formed from the complementary nucleic acids which are hybridized to the target nucleotide sequence. After exonuclease treatment, a T7 promoter oligonucleotide is hybridized to the ligation product and an in vitro transcription reaction is performed to generate a transcript comprising the tag indicative of the presence of the target nucleic acid in the sample. The transcript is hybridized to a chip and detected.

FIG. 5. FIG. 5 illustrates one embodiment of a method in which both the first complementary nucleic acid and the second complementary nucleic acid are on a single molecule which also includes a T7 promoter and a nucleotide sequence complementary to a tag to be used to indicate the presence of a target sequence in, the sample. Sequestering agents which interact with the first and second complementary nucleic acids are provided. Duplexes between the first and second complementary nucleic acids and the target nucleotide sequence are in equilibrium with duplexes between the first and second complementary nucleic acids and the first and second sequestering agents respectively. Duplexes between the first and second complementary nucleic acids and the target nucleotide sequence predominate since they are thermodynamically favored over duplexes between the first and second complementary nucleic acids and the first and second sequestering agents respectively. In addition, in the depicted embodiment, the sequestering agents block access to the reactive groups for ligation on the 3′ and 5′ ends of the complementary nucleic acids, thereby reducing non-templated ligation (ligation of complementary nucleic acids which are not hybridized to the target nucleic acid). If the sample contains the target nucleic acid, a circular ligation product is formed from the complementary nucleic acids which are hybridized to the target nucleotide sequence. After exonuclease treatment, the circular ligation product is cleaved to generate a linear molecule. A T7 promoter oligonucleotide is hybridized to the linearized ligation product and an in vitro transcription reaction is performed to generate a transcript comprising the tag indicative of the presence of the target nucleic acid in the sample. The transcript is hybridized to a chip and detected.

FIG. 6. FIG. 6 illustrates one embodiment of a method in which both the first complementary nucleic acid and the second complementary nucleic acid are on a single molecule which also includes a nucleotide sequence complementary to a tag to be used to indicate the presence of a target sequence in the sample. Sequestering agents which interact with the first and second complementary nucleic acids are provided. Duplexes between the first and second complementary nucleic acids and the target nucleotide sequence are in equilibrium with duplexes between the first and second complementary nucleic acids and the first and second sequestering agents respectively. Duplexes between the first and second complementary nucleic acids and the target nucleotide sequence predominate since they are thermodynamically favored over duplexes between the first and second complementary nucleic acids and the first and second sequestering agents respectively. In addition, in the depicted embodiment, the sequestering agents block access to the reactive groups for ligation on the 3′ and 5′ ends of the complementary nucleic acids, thereby reducing non-templated ligation (ligation of complementary nucleic acids which are not hybridized to the target nucleic acid). If the sample contains the target nucleic acid, a circular ligation product is formed from the complementary nucleic acids which are hybridized to the target nucleotide sequence. After exonuclease treatment, the circular ligation product is cleaved to generate a linear molecule. The linear ligation product is hybridized to a chip and detected.

FIG. 7. FIG. 7 illustrates one embodiment of a method in which both the first complementary nucleic acid and the second complementary nucleic acid are on a single molecule which also includes a T7 promoter and a nucleotide sequence complementary to a tag to be used to indicate the presence of a target sequence in the sample. Sequestering agents which interact with the first and second complementary nucleic acids are provided. Duplexes between the first and second complementary nucleic acids and the target nucleotide sequence are in equilibrium with duplexes between the first and second complementary nucleic acids and the first and second sequestering agents respectively. Duplexes between the first and second complementary nucleic acids and the target nucleotide sequence predominate since they are thermodynamically favored over duplexes between the first and second complementary nucleic acids and the first and second sequestering agents respectively. In addition, in the depicted embodiment, the sequestering agents block access to the reactive groups for ligation on the 3′ and 5′ ends of the complementary nucleic acids, thereby reducing non-templated ligation (ligation of complementary nucleic acids which are not hybridized to the target nucleic acid). If the sample contains the target nucleic acid, a circular ligation product is formed from the complementary nucleic acids which are hybridized to the target nucleotide sequence. After exonuclease treatment, PCR amplification is performed to generate an amplification product containing the T7 promoter and the tag. In vitro transcription is performed with the amplification product and the resulting transcript, containing the tag, is hybridized to a chip and detected.

FIG. 8. FIG. 8 illustrates one embodiment of a method in which both the first complementary nucleic acid and the second complementary nucleic acid are on a single molecule which also includes a nucleotide sequence complementary to a tag to be used to indicate the presence of a target sequence in the sample. Sequestering agents which interact with the first and second complementary nucleic acids are provided. Duplexes between the first and second complementary nucleic acids and the target nucleotide sequence are in equilibrium with duplexes between the first and second complementary nucleic acids and the first and second sequestering agents respectively. Duplexes between the first and second complementary nucleic acids and the target nucleotide sequence predominate since they are thermodynamically favored over duplexes between the first and second complementary nucleic acids and the first and second sequestering agents respectively. In addition, in the depicted embodiment, the sequestering agents block access to the reactive groups for ligation on the 3′ and 5′ ends of the complementary nucleic acids, thereby reducing non-templated ligation (ligation of complementary nucleic acids which are not hybridized to the target nucleic acid). If the sample contains the target nucleic acid, a circular ligation product is formed from the complementary nucleic acids which are hybridized to the target nucleotide sequence. After exonuclease treatment, PCR amplification is performed to generate an amplification product containing the tag. The resulting amplification product, containing the tag, is hybridized to a chip and detected.

FIG. 9. FIG. 9 illustrates one embodiment of a method in which the first complementary nucleic acid and the second complementary nucleic acid are on separate molecules. The first complementary nucleic acid also includes a T7 promoter 5′ of the region which is complementary to the target nucleic acid. The second complementary nucleic acid includes a nucleotide sequence complementary to a tag to be used to indicate the presence of the target nucleic acid in the sample. The nucleotide sequence complementary to the tag is located 3′ of the region which is complementary to the target nucleic acid.

Genomic DNA is amplified by PCR using one primer with a phosphorothioate at the 5′ end and another primer without a phosphorothioate. The amplification product is treated with exonuclease to remove one strand of the amplification product.

The first complementary nucleic acid and the second complementary nucleic acid, which are on separate molecules, are provided. Sequestering agents which interact with the first and second complementary nucleic acids are also provided. Duplexes between the first and second complementary nucleic acids and the target nucleotide sequence are in equilibrium with duplexes between the first and second complementary nucleic acids and the first and second sequestering agents respectively. Duplexes between the first and second complementary nucleic acids and the target nucleotide sequence predominate since they are thermodynamically favored over duplexes between the first and second complementary nucleic acids and the first and second sequestering agents respectively. In addition, in the depicted embodiment, the sequestering agents block access to the reactive groups for ligation on the 3′ and 5′ ends of the complementary nucleic acids, thereby reducing non-templated ligation (ligation of complementary nucleic acids which are not hybridized to the target nucleic acid). The first and second complementary nucleic acids which are hybridized to the target nucleotide sequence If the sample contains the target nucleic acid, a ligation product is formed from the complementary nucleic acids which are hybridized to the target nucleotide sequence. After exonuclease treatment, an oligonucleotide complementary to the T7 promoter is hybridized to the ligation product and in vitro transcription is performed to generate a transcript containing the tag. The transcript is hybridized to a chip and detected.

FIG. 10. FIG. 10 illustrates one embodiment of a method in which the first complementary nucleic acid and the second complementary nucleic acid are on separate molecules. The second complementary nucleic acid includes a nucleotide sequence complementary to a tag to be used to indicate the presence of the target nucleic acid in the sample. The nucleotide sequence complementary to the tag is located 3′ of the region which is complementary to the target nucleic acid.

Genomic DNA is amplified by PCR using one primer with a phosphorothioate at the 5′ end and another primer without a phosphorothioate. The amplification product is treated with exonuclease to remove one strand of the amplification product.

The first complementary nucleic acid and the second complementary nucleic acid, which are on separate molecules, are provided. Sequestering agents which interact with the first and second complementary nucleic acids are also provided. Duplexes between the first and second complementary nucleic acids and the target nucleotide sequence are in equilibrium with duplexes between the first and second complementary nucleic acids and the first and second sequestering agents respectively. Duplexes between the first and second complementary nucleic acids and the target nucleotide sequence predominate since they are thermodynamically favored over duplexes between the first and second complementary nucleic acids and the first and second sequestering agents respectively. In addition, in the depicted embodiment, the sequestering agents block access to the reactive groups for ligation on the 3′ and 5′ ends of the complementary nucleic acids, thereby reducing non-templated ligation (ligation of complementary nucleic acids which are not hybridized to the target nucleic acid). If the sample contains the target nucleic acid, a ligation product is formed from the complementary nucleic acids which are hybridized to the target nucleotide sequence. After exonuclease treatment, the ligation product is hybridized to a chip and detected.

FIG. 11. FIG. 11 illustrates one embodiment of a method in which the first complementary nucleic acid and the second complementary nucleic acid are on separate molecules. The first complementary nucleic acid also includes a T7 promoter 5′ of the region which is complementary to the target nucleic acid. The second complementary nucleic acid includes a nucleotide sequence complementary to a tag to be used to indicate the presence of the target nucleic acid in the sample. The nucleotide sequence complementary to the tag is located 3′ of the region which is complementary to the target nucleic acid.

Genomic DNA is obtained. The first complementary nucleic acid and the second complementary nucleic acid, which are on separate molecules, are provided. Sequestering agents which interact with the first and second complementary nucleic acids are also provided. Duplexes between the first and second complementary nucleic acids and the target nucleotide sequence are in equilibrium with duplexes between the first and second complementary nucleic acids and the first and second sequestering agents respectively. Duplexes between the first and second complementary nucleic acids and the target nucleotide sequence predominate since they are thermodynamically favored over duplexes between the first and second complementary nucleic acids and the first and second sequestering agents respectively. In addition, in the depicted embodiment, the sequestering agents block access to the reactive groups for ligation on the 3′ and 5′ ends of the complementary nucleic acids, thereby reducing non-templated ligation (ligation of complementary nucleic acids which are not hybridized to the target nucleic acid). If the sample contains the target nucleic acid, a ligation product is formed from the complementary nucleic acids hybridized to the target nucleotide sequence. After exonuclease treatment, the ligation product is amplified using PCR. An oligonucleotide complementary to the T7 promoter is hybridized to the amplification product and in vitro transcription is performed to generate a transcript containing the tag. The transcript is hybridized to a chip and detected.

FIG. 12. FIG. 12 illustrates one embodiment of a method in which the first complementary nucleic acid and the second complementary nucleic acid are on separate molecules. The second complementary nucleic acid includes a nucleotide sequence complementary to a tag to be used to indicate the presence of the target nucleic acid in the sample. The nucleotide sequence complementary to the tag is located 3′ of the region which is complementary to the target nucleic acid.

Genomic DNA is obtained. The first complementary nucleic acid and the second complementary nucleic acid, which are on separate molecules, are provided. Sequestering agents which interact with the first and second complementary nucleic acids are also provided. Duplexes between the first and second complementary nucleic acids and the target nucleotide sequence are in equilibrium with duplexes between the first and second complementary nucleic acids and the first and second sequestering agents respectively. Duplexes between the first and second complementary nucleic acids and the target nucleotide sequence predominate since they are thermodynamically favored over duplexes between the first and second complementary nucleic acids and the first and second sequestering agents respectively. In addition, in the depicted embodiment, the sequestering agents block access to the reactive groups for ligation on the 3′ and 5′ ends of the complementary nucleic acids, thereby reducing non-templated ligation (ligation of complementary nucleic acids which are not hybridized to the target nucleic acid). If the sample contains the target nucleic acid, a ligation product is formed from the complementary nucleic acids hybridized to the target nucleotide sequence. After exonuclease treatment, the ligation product is amplified using PCR. The ligation product is hybridized to a chip and detected.

FIG. 13. FIG. 13 illustrates one embodiment of a method in which the first complementary nucleic acid and the second complementary nucleic acid are on separate molecules. The first complementary nucleic acid is complementary to the nucleotide sequence immediately 5′ of the polymorphic nucleotide (in this case the T allele) on the genomic DNA. The first complementary nucleic acid also includes a sequence complementary to a portion of an RC probe. The second complementary nucleic acid is complementary to the nucleotide sequence in the genomic DNA which includes the polymorphic nucleotide such that the A which is complementary to the T allele is at the 3′ end of the complementary nucleic acid. The second complementary nucleic acid also comprises a nucleotide sequence complementary to a portion of the RC probe.

A first sequestering agent comprising a nucleotide sequence complementary to a portion of the first complementary nucleic acid and a second sequestering agent comprising a nucleotide sequence complementary to a portion of the second complementary nucleic acid are provided. Duplexes between the first and second complementary nucleic acids and the target nucleotide sequence predominate since they are thermodynamically favored over duplexes between the first and second complementary nucleic acids and the first and second sequestering agents respectively. In addition, in the depicted embodiment, the sequestering agents block access to the reactive groups for ligation on the 3′ and 5′ ends of the complementary nucleic acids, thereby reducing non-templated ligation (ligation of complementary nucleic acids which are not hybridized to the target nucleic acid).

Because the first and second complementary nucleic acids are complementary to adjacent nucleotide sequences in the genomic DNA, when the genomic DNA contains the T allele complementary to the 3′ terminal nucleotide of the second complementary nucleic acid, the first complementary nucleic acid and the second complementary nucleic acid hybridized to the target nucleotide sequence will be ligated together to generate a ligation product.

A 3′ and 5′ exonuclease treatment is used to remove sequestering agents and unligated complementary nucleic acids. A rolling circle probe containing a sequence complementary to the first complementary nucleic acid at one of its termini and a sequence complementary to the second complementary nucleic acid at the other terminus is hybridized to the ligation product. The rolling circle probe also contains a sequence complementary to a tag to be used to indicate the presence of the target nucleic acid in the sample. The rolling circle probe hybridized to the ligation product is ligated to generate a circular molecule. DNA polymerase is provided to extend the ligation product which is hybridized to the circular molecule, generating a product containing repeating units comprising the tag. The product may be trimmed and placed in contact with a detector comprising a detection probe complementary to the tag and hybridization is detected as described above.

FIG. 14. FIG. 14 illustrates the structure and melting temperature of duplexes between a first complementary nucleic acid for detection of the Factor V alleles present in a sample and a first sequestering agent and the structure and melting temperature of duplex between the first complementary nucleic acid and the target nucleotide sequence.

FIG. 15. FIG. 15 illustrates the structure and melting temperature of duplexes between a second complementary nucleic acid for detection of the Factor V alleles present in a sample and a second sequestering agent and the structure and melting temperature of duplex between the second complementary nucleic acid and the target nucleotide sequence.

FIG. 16A. FIG. 16A illustrates an embodiment in which the stability of the duplex between the target and the first complementary nucleic acid is comparable to the stability of the duplex between the target and the second complementary nucleic acid. In particular, FIG. 16A illustrates an embodiment in which the Tm of the duplex between the first complementary nucleic acid and the target is 75.4° C.

FIG. 16B. FIG. 16B illustrates an embodiment in which the stability of the duplex between the target and the first complementary nucleic acid is comparable to the stability of the duplex between the target and the second complementary nucleic acid. In particular, FIG. 16B illustrates an embodiment in which the Tm of the duplex between the second complementary nucleic acid and the target is 77.8° C.

### DETAILED DESCRIPTION OF THE PREFERRED EMBODIMENT

“Stringency clamping” has been employed to maintain specific binding under non-stringent conditions. (See Roberts, R. W. and Crothers, D. M., Proc. Natl. Acad. Sci. USA Vol. 88: 9397-9401, the disclosure of which is incorporated herein by reference in its entirety).

The present invention disclosure provides methods and compositions for detecting the presence of a target nucleic acid in a sample. Further provided are kits containing the foregoing compositions for use in detecting the presence of a target nucleic acid in a sample. The present disclosure provides methods and compositions for detecting target nucleic acids in a sample using sequestering agents which reduce the likelihood of hybridization to non-target nucleic acids present in the sample.

The sample which contains the target nucleic acid may be of any origin. For example, in some embodiments, the sample may be a genomic DNA preparation. Alternatively, in other embodiments, the sample may be an RNA preparation, a cDNA preparation, or a preparation containing an amplified nucleic acid comprising the target nucleic acid.

The target nucleic acid may be any nucleic acid which one may desire to detect. For example, in one embodiment, the target nucleic acid may be a nucleic acid containing at least one polymorphic nucleotide (such as a SNP). In other embodiments, the target nucleic acid may be an allelic variant, a splice variant, a deletion, an insertion, a translocation junction, a chromosomal breakpoint, a repeated sequence or a nucleic acid within the genome of a pathogen or a nucleic acid transcribed from the genome of a pathogen.

In the present methods, at least two nucleic acids (referred to herein as “complementary nucleic acids” because they are complementary to nucleotide sequences in the target nucleic acid) are provided. The complementary nucleic acids may have any length and any nucleotide sequence consistent with their use in the methods described herein.

In some embodiments, the at least two complementary nucleic acids are present in the same nucleic acid molecule, such that ligation of the complementary nucleic acids to one another produces a circular nucleic acid. In other embodiments, the at least two complementary nucleic acids are distinct molecules which are not physically joined to one another prior to ligation. For example, in some embodiments, the at least two complementary nucleic acids are distinct oligonucleotides.

In some embodiments, the at least two complementary nucleic acids are complementary to nucleotide sequences which are immediately adjacent to one another on the target nucleic acid, such that their 5′ and 3′ ends can be directly ligated to one another while they are hybridized to the target nucleic acid. In other embodiments, the at least two complementary nucleic acids are complementary to nucleotide sequences which are not immediately adjacent to one another on the target nucleic acid. In such embodiments, the complementary nucleic acids may be ligated to one another after hybridization to the target nucleic acid and extension using a polymerase, as is often employed in gap LCR procedures. (See Abravaya et al., Nucleic Acids Research 23: 675 (1995) and U.S. Pat. No. 5,427,930, the disclosures of which are incorporated herein by reference in their entireties.)

In embodiments in which the target nucleic acid contains a polymorphic nucleotide, such as when the target nucleic acid is a SNP, one of the complementary nucleic acids may comprise the variable nucleotide such that it is fully complementary to one of the alleles of the SNP. In farther embodiments, the polymorphic nucleotide may be at the 3′ terminus of the complementary nucleic acid. In other embodiments, the polymorphic nucleotide may be at the 5′ terminus of the complementary nucleic acid.

At least one of the complementary nucleic acids, and in some embodiments, both or all of the complementary nucleic acids, is allowed to specifically interact with a sequestering agent which reduces the likelihood that the complementary nucleic acid will hybridize to non-target nucleic acids present in the sample. As used herein, “specific interaction” means that the sequestering agent is adapted to have a greater affinity, preferably a significantly greater affinity, for the complementary nucleic acid with which it is intended to interact than for other nucleic acids. One example of “specific interaction” is duplex formation via nucleic acid base pairing (e.g. the sequestering agent contains a nucleotide sequence which is complementary to at least a portion of the complementary nucleic acid). Non-specific interactions include interactions between an ion, such as a sodium ion, and the complementary nucleic acid. Such an interaction is non-specific because the sodium ion does not have greater affinity for the complementary nucleic acid than for nucleic acids having a different sequence.

Preferably, the stability of the interaction between the complementary nucleic acid and the sequestering agent is greater than the stability of a duplex between the complementary nucleic acid and non-target nucleic acids in the sample, such that the interaction between the complementary nucleic acid and the sequestering agent is favored over a duplex between the complementary nucleic acid and non-target nucleic acids in the sample. In some embodiments, the complex between the complementary nucleic acid and the sequestering agent is at least about 2-fold, at least about 10-fold, at least about 20-fold, or at least about 40-fold more stable than duplexes between the complementary nucleic acid and non-target nucleic acids in the sample. However, it will be appreciated that the stability of the complex between the complementary nucleic acid and the sequestering agent relative to the stability of the complex between the complementary nucleic acid and the non-target nucleic acids may have any value consistent with the methodology described herein. In addition, in a preferred embodiment, the sequestering agents provide a structure that blocks or reduces access to the reactive 3′-OH and 5′-phosphate groups on the complimentary nucleic acids when the complementary nucleic acids are not bound to the target nucleotide sequence, thereby thereby reducing non-templated ligation (ligation of complementary nucleic acids which are not hybridized to the target nucleic acid). The reactive groups are available when the complementary nucleic acid are hybridized to the target sequence.

In some embodiments it may be desirable that the duplex between the target and the first and second complementary nucleic acids have comparable stability. This embodiment allows the duplex between each of the complementary nucleic acids and the target to play a thermodynamic role in aiding the specificity of the overall ligation process. In other embodiments, the duplex between one of the complementary nucleic acids and the target may have a higher stability than the duplex between the target and the other complementary nucleic acid. In further embodiments, the duplex one of the complementary nucleic acids and the target may have a lower stability than the duplex between the target and the other complementary nucleic acid. For example, in some embodiments, the melting temperature of duplexes between the first complementary nucleic acid and the target nucleic acid is less than about 5° C., less than about 4° C., less than about 3° C., less than about 2° C., or less less than about 1° C. different from the melting temperature of duplexes between the second complementary nucleic acid and the target nucleic acid.

In other embodiments, the melting temperature of duplexes between the first complementary nucleic acid and the target nucleic acid is at least about 5° C. greater, at least about 8° C. greater, at least about 10° C. greater, or at least about 15° C. greater than the melting temperature of duplexes between the second complementary nucleic acid and the target nucleic acid.

In other embodiments, the melting temperature of duplexes between the second complementary nucleic acid and the target nucleic acid is at least about 5° C. greater, at least about 8° C. greater, at least about 10° C. greater, or at least about 15° C. greater than the melting temperature of duplexes between the first complementary nucleic acid and the target nucleic acid.

Sequestering agents reduce the likelihood that the complementary nucleic acids will hybridize with non-target nucleic acids by specifically interacting with the complementary nucleic acids. In a preferred embodiment, the sequestering agent is a nucleic acid which contains a nucleotide sequence which is complementary to at least a portion of the complementary nucleic acid with which it is to interact such that the complementary nucleic acid and the sequestering agent can form a duplex.

In a preferred embodiment, the sequestering agents provide a structure that blocks or reduces access to the reactive 3′-OH and 5′-phosphate groups on the complimentary nucleic acids when the complementary nucleic acids are not bound to the target nucleotide sequence, thereby thereby reducing non-templated ligation (ligation of complementary nucleic acids which are not hybridized to the target nucleic acid). Thus, in such embodiments, the reactive groups are available when the complementary nucleic acids are hybridized to the target sequence. For example, in some embodiments, the reactive groups on complementary nucleic acids which are not hybridized to the target nucleotide sequence are blocked by a hairpin helix at the 3′ or 5′ end of the sequestering agent. In some embodiments, the 3′ terminal nucleotide of the sequestering agent lacks a hydroxyl group such that a phosphate on the 5′ nucleotide of the complementary nucleic acid cannot be ligated thereto when the complementary nucleic acid is hybridized to the sequestering agent. For example, in some embodiments, the nucleotide at the 3′ terminus of the sequestering agent may have a dideoxy sugar. Alternatively, the nucleotide at the 3′ terminus of the sequestering agent may comprise a blocking group which prevents ligation of a 5′ phosphate on the complementary nucleic acid to the 3′ terminus of the sequestering agent when the complementary nucleic acid is hybridized to the sequestering agent. For example, the blocking group may be F, Cl, Br, NO₂, OR₁, O—C(O)—R₂, NHR₃, alkyl, and H where R₁ is selected from the group consisting of carbonyl, alkyl, acyl, alkene, or heteroalkyl, said alkyl, acyl, alkene or heteroalkyl group being substituted or unsubstituted, R₂ is selected from the group consisting of alkyl, acyl, alkene, or heteroalkyl, said alkyl, acyl, alkene or heteroalkyl group being substituted or unsubstituted and R3 is selected from the group consisting of allyl, acyl, alkene, or heteroalkyl, said alkyl, acyl, alkene or heteroalkyl group being substituted or unsubstituted. However, it will be appreciated that any blocking group which prevents or inhibits ligation of a 5′ phosphate on the complementary nucleic acid to the 3′ terminus of the sequestering agent when the complementary nucleic acid is hybridized to the sequestering agent may be used.

In other embodiments, the 5′ terminal nucleotide of the sequestering agent may lack a 5′ phosphate such that the 5′ terminal nucleotide cannot be ligated to another nucleic acid through the activity of a ligase. In some embodiments, the 5′ terminal nucleotide of the sequestering agent may comprise a blocking group which prevents ligation to other nucleic acids. In some embodiments, the nucleotide at the 5′ terminus of the sequestering agent may comprise a blocking group which prevents ligation of a 3′ hydroxyl on another nucleic acid to the 5′ terminus of the sequestering agent. For example, the blocking group may be F, Cl, Br, NO₂, OR₁, O—C(O)—R₂, NHR₃, alkyl, and H where R₁ is selected from the group consisting of carbonyl, alkyl, acyl, alkene, or heteroalkyl, said alkyl, acyl, alkene or heteroalkyl group being substituted or unsubstituted, R₂ is selected from the group consisting of alkyl, acyl, alkene, or heteroalkyl, said alkyl, acyl, alkene or heteroalkyl group being substituted or unsubstituted and R3 is selected from the group consisting of alkyl, acyl, alkene, or heteroalkyl, said alkyl, acyl, alkene or heteroalkyl group being substituted or unsubstituted. However, it will be appreciated that any blocking group which prevents or inhibits ligation of a 3′ hydroxyl on another nucleic acid to the 5′ terminus of the sequestering agent may be used.

In a preferred embodiment, the sequestering agent is a nucleic acid which includes a stem loop. For example, in some embodiments, the 3′ terminus of the sequestering agent is in the duplex portion of the stem loop. Preferably, the stem loop has a melting temperature of at least about 55° C., at least about 65° C., at least about 75° C. or at least about 85° C. Preferably, the stem loop sequences do not share complementarity with sequences in the target or complementary nucleic acids. Hybridization of the complementary nucleic acid to the sequestering agent near the terminus of the hairpin reduces the likelihood that the complementary nucleic acid will undergo non-specific ligation to another molecule, since the end of the complementary nucleic acid which is near the terminus of the hairpin is not accessible to other nucleic acids.

In other embodiments, the sequestering agent may be a nucleic acid which has a linear conformation.

In preferred embodiments, the sequestering agent reduces the accessibility of the 5′ phosphate of the complementary nucleic acid to a ligase when the complementary nucleic acid is hybridized to the sequestering agent.

Preferably, the stability of the interaction between the complementary nucleic acid and its complementary nucleotide sequence on the target nucleic acid is greater than the stability of a duplex between the complementary nucleic acid and the sequestering agent, such that the interaction between the complementary nucleic acid and the complementary nucleotide sequence in the target nucleic acid is favored over a duplex between the complementary nucleic acid and the sequestering agent. In some embodiments, the complex between the complementary nucleotide sequence in the target nucleic acid is at least about two-fold, at least about five-fold, at least about ten-fold, or at least about 20-fold less stable than complexes between the complementary nucleic acid and the sequestering agent. In some embodiments, the duplex between the complementary nucleic acid and its complementary nucleotide sequence in the target nucleic acid has a melting temperature which is at least about 5 degrees Celsius, at least about 10 degrees Celsius, or at least about 15 degrees Celsius higher than the melting temperature of the duplex between the complementary nucleic acid and the sequestering agent.

For example, in some embodiments, the complex between the sequestering agent and the complementary nucleic acid has a melting point intermediate to the melting point of a duplex between the complementary nucleic acid and non-target nucleic acids in the sample and the melting point of a duplex between the complementary nucleic acid and the complementary nucleotide sequence in the target nucleic acid.

Because any complementary nucleic acid which is not hybridized to the target nucleotide sequence will be hybridized to the sequestering agent rather than to non-target nucleic acids in the sample, the hybridization of the complementary nucleic acids to the sample nucleic acids may be conducted under non-stringent conditions. Thus, in some embodiments, the hybridization of the complementary nucleic acid to the sample nucleic acids may be conducted at temperatures at least 5 degrees Celsius below the melting temperature of the duplex between the complementary nucleic acid and the target nucleotide sequence, at least 10 degrees Celsius below the melting temperature of the duplex between the complementary nucleic acid and the target nucleotide sequence, at least 15 degrees Celsius below the melting temperature of the duplex between the complementary nucleic acid and the target nucleotide sequence, at least 20 degrees Celsius below the melting temperature of the duplex between the complementary nucleic acid and the target nucleotide sequence, or at least 25 degrees Celsius below the melting temperature of the duplex between the complementary nucleic acid and the target nucleotide sequence. If desired, salt concentration may also be varied to establish non-stringent conditions, such that the ligation reaction occurs well below the point of instability of the duplex between the complementary nucleic acid and the target nucleotide sequence.

In some embodiments it may be desirable that the duplex between the target and the first and second complementary nucleic acids have comparable stability. This embodiment allows the duplex between each of the complementary nucleic acids and the target to play a thermodynamic role in aiding the specificity of the overall ligation process. In other embodiments, the duplex between one of the complementary nucleic acids and the target may have a higher stability than the duplex between the target and the other complementary nucleic acid. In further embodiments, the duplex one of the complementary nucleic acids and the target may have a lower stability than the duplex between the target and the other complementary nucleic acid. For example, in some embodiments, the melting temperature of duplexes between the first complementary nucleic acid and the target nucleic acid is less than about 5° C., less than about 4° C., less than about 3° C., less than about 2° C., or less less than about 1° C. different from the melting temperature of duplexes between the second complementary nucleic acid and the target nucleic acid.

In other embodiments, the melting temperature of duplexes between the first complementary nucleic acid and the target nucleic acid is at least about 5° C. greater, at least about 8° C. greater, at least about 10° C. greater, or at least about 15° C. greater than the melting temperature of duplexes between the second complementary nucleic acid and the target nucleic acid.

In other embodiments, the melting temperature of duplexes between the second complementary nucleic acid and the target nucleic acid is at least about 5° C. greater, at least about 8° C. greater, at least about 10° C. greater, or at least about 15° C. greater than the melting temperature of duplexes between the first complementary nucleic acid and the target nucleic acid.

If the target nucleic acid is present in the sample, the complementary nucleic acids hybridize to their complementary sequences in the target nucleic acid. If the sample contains the target nucleic acid, the hybridized complementary nucleic acids are ligated together to generate a ligation product. The ligation product is detected directly or indirectly to indicate the presence of the target nucleic acid in the sample.

The concentrations of the complementary nucleic acids and the sequestering agent used in the methods described herein may be any concentrations which allow the target nucleic acid to be detected if present. In some embodiments, the concentrations of the complementary nucleic acids and sequestering agent(s) are selected so that at equilibrium about 0.5% or less of the nucleotide sequences in the target nucleic acid which are recognized by the complementary nucleic acids is unoccupied by the complementary nucleic acid. In other embodiments, the concentrations of the complementary nucleic acids and sequestering agent(s) are selected so that at equilibrium more than about 20%, less than about 20%, less than about 10%, less than about 5%, or less than about 1% of the nucleotide sequences in the target nucleic acid which are recognized by the complementary nucleic acids is unoccupied by the complementary nucleic acid. Occupancy levels may be determined using the following equilibrium equations:

$$target + probe - suppressor ⇄ probe - target + suppressor$$

$$K = \frac{[ probe - target ] [ suppressor ]}{[ target ] [ probe - suppressor ]}$$

where K is much greater than 1. Rearranging this equation gives

$$\frac{[ target ]}{[ probe - target ]} = \frac{1}{K} \frac{[ suppressor ]}{[ probe - suppressor ]}$$

For example, assuming that the probe and sequestering agents are present in much higher concentration than the target, addition of sequestering agents at 1.5 molar excess will yield a ratio of sequestering agents to probe-sequestering agent concentration of 0.5. If K is 20, the ratio of target to probe target will be 0.025. In this case, 2.5% of the target molecules are not occupied by probe. In some embodiments, the sequestering agent is present in a molar excess of about 1.1 to about 2 relative to the complementary nucleic acid. In other embodiments, the sequestering agent is present in a molar excess of about 2.5, about 3, about 5, about 10, or more than 10 relative to the complementary nucleic acid. Thus, in some embodiments, the concentration of sequestering agent is selected such that only a small percentage (for example, <10%, <20%, or <30%) of the target is not hybridized to probe. The optimal concentration depends on the value of K.

In some embodiments, the ligation product or a molecule derived from the ligation product comprises an identifier tag which can hybridize to a complementary sequence on a universal chip. Thus, in some embodiments, one or more of the complementary nucleic acids used to generate the ligation product comprises an identifier tag. In alternative embodiments, the ligation product itself does not contain an identifier tag, but rather, the ligation product is used to generate a molecule which comprises an identifier tag. In such embodiments, the tagged molecules having identifier tags are incubated with a universal detector having detection probes coupled to a detector or coupled to detection means, and hybridization of a particular identifier tag to its complementary detection probe indicates the presence of the corresponding target in the sample being assayed. In the methods described herein, target-dependent procedures are used to generate tagged molecules, advantageously increasing accuracy and minimizing spurious signals without the need to employ special conditions or special reagents. The universal tag assay can easily be used to assay a wide variety of samples. The universal tag assay can be performed in a single vessel and easily be automated. For example, the ligation products described herein may be used in universal tag assays such as those described in U.S. Provisional Patent Application Ser. No. 60/424,656, entitled Universal Tag Assay, filed Nov. 6, 2002, the disclosure of which is incorporated herein by reference in its entirety.

It will be appreciated that, although many of the embodiments discussed herein and illustrated in the figures utilize universal chips and identifier tags, those are only particular embodiments of the present invention. Other embodiments which do not utilize universal chips and identifier tags may readily be practiced by simply omitting the identifier tags or sequences complementary thereto from the nucleic acids discussed below and hybridizing rolling circle amplification products, PCR products, or other nucleic acids indicative of the presence of a target nucleic acid in a sample to a chip containing a nucleic acid complementary comprising at least a portion of the target nucleic acid or a sequence complementary thereto, such that hybridization to the chip indicates the presence of the target nucleic acid in the sample.

The term “target nucleic acid” as used herein refers to a nucleic acid which one desires to detect the presence or absence of in a sample. As discussed above, the sample may be any sample containing nucleic acids, and, in some embodiments, may be a sample comprising genomic DNA, RNA, cDNA, or a nucleic acid obtained through an amplification procedure.

A “nucleotide sequence in a target nucleic acid” or a “target nucleotide sequence” is a nucleotide sequence in the target nucleic acid which is the complement of a complementary nucleic acid used in the present methods. In embodiments in which multiple target nucleic acids are being assayed, the target nucleotide sequences in these target nucleic acids which are the complements of the complementary nucleic acids may be the same or different lengths.

In an illustrative embodiment, a gene has a single nucleotide polymorphism (SNP) with two variant sequences, wherein each variant sequence is associated with a particular phenotype or a probability of having a certain characteristic or phenotype. In such an embodiment, a complementary nucleic acid which is capable of distinguishing which variant of the SNP is present in the sample is utilized. For example, in some embodiments, including embodiments in which the first and second complementary nucleic acids are present on a single nucleic acid molecule, the complementary nucleic acids may be at least about 60, at least about 80, at least about 100 or more than 100 nucleotides in length. Alternatively, in some embodiments, including emobdiments in which the first and second complementary nucleic acids are on separate nucleic acid molecules, the compmentary nucleic acids are each at least about 15, at least about 20, at least about 25, at least about 30, at least about 35, at least about 40, or more than 40 nucleotides in length. In some embodiments, one of the complementary nucleic acids contains one of the alleles of the polymorphic nucleotide, preferably at one of its termini. In some embodiments, one of the complementary nucleic acids contains the polymorphic nucleotide at its 3′ terminus. In other embodiments, one of the complementary nucleic acids contains the polymorphic nucleotide at its 5′ terminus.

In some embodiments, two or more complementary nucleic acids each of which includes one of the alleles of the polymorphic nucleotide are added to the sample to be analyzed. In some embodiments, two or more sequestering agents each of which is fully complementary to at least a portion of one of the complementary nucleic acids which includes the polymorphic nucleotide are added to the sample.

In some embodiments, a tagged molecule indicative of the presence of a target nucleic acid in the sample is generated. A “tagged molecule” contains an identifier tag for a particular target nucleic acid and may optionally contain a copy or complement of the target nucleic acid or a portion thereof. A tagged molecule may contain additional sequence. A tagged molecule is a molecule that interacts with the universal detector as follows: the tagged molecule containing an identifier tag is incubated with the universal detector having detection probes, and the identifier tag in the tagged molecule hybridizes to a complementary detection probe of the universal detector. Tagged molecules are generated by target-dependent processes, such that a tagged molecule containing an identifier tag is generated only when the target nucleic acid corresponding to that identifier tag is present in the sample. The tag may be present on the ligation product comprising the first complementary nucleic acid or, alternatively, may be introduced in a procedure which is dependent upon having generated the ligation product comprising the first complementary nucleic acid and the second complementary nucleic acid.

Preferably, an “identifier tag” is an oligonucleotide having a known nucleotide sequence called the “tag sequence” or “identifier tag sequence.” A tagged molecule may be a “tagged target nucleic acid” that contains a copy or complement of the target nucleic acid and an identifier tag for that target nucleic acid. Alternately, a tagged molecule may contain an identifier tag for a particular target nucleic acid and no copy or complement of the corresponding target nucleic acid. A tagged molecule having only the identifier tag and no copy or complement of the target nucleic acid may be generated by cleaving the products of various target-dependent processes to release tagged molecules having only the identifier tag. Alternately, a tagged molecule having only the identifier tag and no copy or complement of the target nucleic acid may be generated by target-dependent processes that only generate copies of the identifier tag. In one embodiment, target nucleotide sequence is amplified, and tagged amplification products having at least one copy of the target and at least one identifier tag are trimmed to generate a smaller tagged molecule containing only the identifier tag. In another embodiment, target-dependent binding of a primer or probe generates a tagged product that can be trimmed to release an identifier tag. An identifier tag suitable for use in the universal tag assay is generated only when the corresponding target is present in a sample being interrogated. Thus, a tagged molecule that contains an identifier tag and does not contain a copy or complement of the target is sufficient to indicate the presence of the corresponding target in the sample being assayed.

Identifier tags in tagged molecules suitable for use with a universal detector of the universal tag assay may be DNA or RNA oligonucleotides, and may include modified bases, non-naturally bases, and labels. Generally, tagged molecules are oligonucleotides or polynucleotides (depending on length) that may include modified bases or non-naturally occurring bases, and may additionally include labels, ligands or other materials and modifications suitable to a particular application. Tagged molecules for use with a universal detector can be generated from any suitable template including but not limited to genomic DNA, cDNA, PCR products, LCR products, RC amplification products, synthetic DNA, other forms of DNA, mRNA, rRNA, synthetic RNA, and other forms of RNA. Advantageously, the use of identifier tags and a universal detector having complementary detection probes provides a universal tag assay that is independent of the organism or tissue being analyzed. Multiple target nucleotide sequences can be detected simultaneously, due to the one-to-one correspondence between each identifier tag and the target nucleotide sequence for which it serves as an identifier, and due to the specificity of hybridization of each identifier tag to its detection probe.

A. Tags and Probes

One aspect of the invention provides a set of tags and probes for use in accordance with the methods and compositions herein disclosed. Detection probes used with universal detectors of the present invention are directed to complementary tags that serve as identifiers for target nucleic acids. Likewise, tags that serve as identifiers for target nucleic acids are directed to complementary detection probes used with universal detectors of the present invention. Hybridization of a tag to its complementary detection probe on a universal detector generates a signal that indicates the presence of the corresponding target nucleic acid known to be identified by that tag. Accordingly, a sample can be interrogated for the presence of target nucleic acids of interest using tags and probes of the present invention as follows: a) tags are chosen such that each tag serves as an identifier tag for one target nucleic acid; b) a tag capable of hybridizing to a complementary detection probe will be generated only if the sample being interrogated contains the particular target nucleic acid for which that tag serves as an identifier tag; and c) only a tag generated as a result of the presence of the corresponding target nucleic acid in the sample will hybridize to a detection probe and generate a signal on a universal detector.

One of skill in the art would understand that a set of tags and probes is chosen such that each tag to be used as an identifier tag in a particular application has a complementary detection probe on the universal detector being used in that application. One of skill would also understand that the universal tag assay may be practiced using a set of detection probes that includes detection probes complementary to tags that are not being used in a particular application. For example, a universal detector may advantageously be manufactured with a fixed array of 1000 detection probes for use in a wide variety of applications, while a particular application using that universal detector may only use 50-100 identifier tags to interrogate a sample.

It is understood that not only does measuring hybridization of a tag to its complementary detection probe reliably indicate the presence of the corresponding target nucleic acid in a sample, but the absence of hybridization of a tag to its complementary detection probe can also reliably indicate the absence of the corresponding target nucleic acid in a sample. Preferably, at least one internal control is included in a universal tag assay, such that reliably obtaining the expected result from the internal control(s) supports the reliability of results indicating the either the presence or absence of a tag hybridization signal. Multiple internal controls may be used to increase the reliability and robustness of an assay.

Tag/probe sets may include control sequences that may be used for calibration, quality control, and comparison between experiments. Control sequences may include constant sequences or “housekeeping” sequences that are expected to be present in a sample and produce tagged molecules. If desired, the robustness of the assay may be enhanced by choosing more than one distinct tag to serve as an identifier tag for the same target nucleic acid. Advantageously, hybridization of all identifier tags corresponding to the same target nucleic acid to their complementary detection probes would more reliably indicate the presence of the target nucleic acid in the sample being assayed. Likewise, if none of the identifier tags corresponding to the same target nucleic acid hybridize to their complementary tags (especially if other internal controls give positive hybridization signals that indicate suitable reaction conditions), then such a signal more reliably indicates the absence of the target nucleic acid in the sample being assayed. Intermediate results wherein only a few of the identifier tags bind could serve as a signal that reagents or reaction conditions should be examined.

As used herein, the term “tag” generally refers to a molecule capable of binding to a probe, where “tag” may encompass tag molecules attached to a target molecule, tag molecules not attached to target molecules, tags expressed in computer-readable form, and the concept of tags as disclosed herein. The term “tag sequence” as used herein refers to the nucleotide sequence of an oligonucleotide tag, where “tag sequence” or “identifier tag sequence” may describe a string of nucleotides or may describe an information string representing the properties of the string of nucleotides, where such an information string can be manipulated as part of a program for designing or selecting a set of tags having desired properties; preferably, the information string is in computer-readable form. In the present invention, an “identifier tag” is a tag chosen to serve as a distinct identifier for a particular target. As used herein, the term “identifier tag” is used to refer both to the oligonucleotide that binds to a complementary detection probe and to nucleotide sequence of the identifier tag. The term “complement of an identifier tag” can refer to a string of nucleotides that make up the oligonucleotide having a nucleotide sequence complementary to the nucleotide sequence of the identifier tag, and can also refer to the nucleotide sequence (information string) of the complement.

As used herein, the term “detection probe” generally refers to a molecule capable of binding to a tag, where “detection probe” may encompass probe molecules immobilized to a support, probe molecules not immobilized to a support, probes expressed in computer-readable form, and the concept of detection probes as disclosed herein. More specifically, the term “probe sequence” as used herein refers to the nucleotide sequence of an oligonucleotide probe, where “probe sequence” may describe a physical string of nucleotides that make up a sequence, or may describe an information string representing the properties of the string of nucleotides, where such an information string can be manipulated as part of a program for designing or selecting a set of probes having desired properties; preferably, the information string is in computer-readable form. The term “detection probe” is generally used herein to refer to a tag-complementary probe coupled to a detection means for measuring hybridization of a tag to the detection probe. Preferably, a detection probe is immobilized to a support that includes a detection means. Such a support may include but is not limited to a surface, a film, or a particle, where a surface is preferably a “chip” surface suitable for mounting an array of immobilized probes and having at least one component of the detection means, and a particle is preferably a bead having at least one component of the detection means. “Detection probe” can also refer to a computational model of a tag-complementary probe coupled to a detection means for detecting hybridization. The term “detection probe” may particularly be used herein to distinguish the detection probe from other components also referred to by the term “probe” e.g., RC probes and RC padlock probes.

In accordance with one aspect of the present invention, a set of tag sequences and probe sequences is selected such that a tag having a certain tag sequence will hybridize only to a probe having a sequence that is an exact complement, and no tag will detectably hybridize with any other probe in the set that is not its exact complement. Such a set is referred to herein as a “minimally cross-hybridizing set.” It is understand that due to complementarity, a minimally cross-hybridizing set of tag sequences and probe sequences may be selected on the basis of tag sequence or probe sequence. Preferably, all tag sequences in a set are selected to have the same or substantially the same G-C content, such that all probe/tag duplexes have similar melting temperatures. Preferably, tag sequences are selected such that all probe/tag duplexes have similar stacking energy. Advantageously, such a set will provide tag-probe hybridization reactions with the desired level of selectivity. Even more preferably, such selective hybridization reactions can be carried out under conditions of moderate stringency.

The length of tag (and probe) sequences suitable for a given embodiment can be determined by one of skill in the art. Preferably, the length of tag and probe sequences is determined by the size of the tag/probe set used to interrogate a sample. Generally, the size of the tag/probe set used to interrogate a sample will determine the degree of complexity needed, and tag/probe length is an important determinant of complexity. Generally, the estimated number of targets being tested in a sample will determine the size of the tag/probe set needed for that embodiment. A set of tags and probes suitable for use in the universal chip system may include tags and probes of different lengths, as long as all tags and probes satisfy the hybridization criteria for a given embodiment For embodiments involving low density arrays wherein about 100 or fewer targets are to be detected, tags having a length of 10, 11, 12, 13, 14, 15, 16, 17, or 18 nucleotides may be utilized. Preferably, a tag sequence for a low-density array is 15 nucleotides in length. Tags longer than 18 nucleotides may be used for low density arrays if desired. For embodiments involving higher density arrays wherein hundreds or thousands of target sequences are to be detected, tag and probe sequences may need to be greater than 15 nucleotides in length, in order to provide a sufficiently large set of tags and probes that satisfy the hybridization criteria for a given embodiment.

Algorithms for generating minimally cross-hybridizing sets of tags and probes are known in the art. A set of tags and probes having desired properties may be obtained by following some or all of a series of tag selection steps, as follows: a) determining all possible tag sequences of a selected length, and/or all possible tag sequences with selected hybridization properties b) selecting tag sequences so that all tags differ by at least two nucleotides in the tag sequence string, such that no tag can hybridize to a non-complementary probe with fewer than two mismatches; c) if desired, refining the selection based on the relative destabilizing effects of mismatches at different positions; d) selecting tag sequences so that there is no secondary structure within the complementary probes used to detect the tags; e) selecting tags so that probes complementary to the tags do not hybridize to each other; f) when all tags are the same length, selecting tags so that all tags have substantially the same, and preferably exactly the same, overall base composition (i.e., the same A+T to G+C ratio), so all tag/probe pairs have the same melting temperature; g) when tags are differing lengths, selecting tags having the A+T to G+C ratio that permits all tag/probe pairs to have the same melting temperature. Additional steps not recited here may also be appropriate to obtain a set of tags and probes having desired properties suitable for a particular embodiment.

Selection steps such as those recited above may be performed in various art-recognized ways. Approaches to designing tag/probe sets for use in a particular application include computational “in silico” approaches to model tag and probe behavior, or experimental “in vitro” approaches using biomolecules such as polynucleotides to accomplish tag and probe sorting, or combinations of these approaches.

Computational approaches can be used in which computational algorithms serve as models of biological molecules. Such approaches and algorithms are known in the art. For example, computer programs installed on computers can be used to make the relevant calculations and comparisons, to execute a desired set of selection steps, and to generate a suitable set of sequence tags. Methods for applying a series of selection steps to design a tag/probe set can be found in the art, e.g., as disclosed by Morris et al. U.S. Pat. No. 6,458,530 and EP 0799897) where a pool of potential tags is generated and a series of pairwise comparisons is carried out to yield a final set of tags that satisfy certain selection criteria. Open-ended computational approaches such as genetic algorithms to generate (locally) optimized populations may be used.

In a preferred embodiment, a universal chip for use in the universal tag assay includes an array of electrically coupled detection probe sequences lacking G (guanosine) bases, thereby permitting electrochemical detection of hybridization of tagged DNA or RNA molecules by detecting G oxidation in tagged molecules (containing G) bound to detection probes, using methods for detecting oxidation-reduction known in the art. For example, G-oxidation in tagged molecules may be detected using transition metal complexes, preferably ruthenium complexes, as disclosed in U.S. Pat. No. 5,871,918. Advantageously, the use of redox-inactive detection probes (e.g., probes lacking G) permits a high density of probes on a universal detector without a background oxidation signal.

B. Universal Detector

An object of the present invention provides a universal detector having detection probes complementary to identifier tags, where detection probes are coupled to a detection means and the interaction of identifier tags with complementary detection probes indicates the presence or absence of targets in the sample being interrogated. Preferably, a universal detector has an array of detection probes. An “array” is a collection of probes in a known arrangement, and an “array of detection probes” as disclosed herein provides a medium for detecting the presence of targets in a sample based on rules for matching tags and probes, where the rules for matching tags and probes are peculiar to each embodiment. Generally, an array of detection probes refers to an array of probes immobilized to a support, where the sequence (the identity) of each detection probe at each location is known. Alternately, an array of detection probes may refer to a set of detection probes that are not immobilized and can be moved on a surface, or may refer to a set of detection probes coupled to one or more particles such as beads. Preferably, the process of detecting and identifier tags hybridized to detection probes is automated. Microarrays having a large of number of immobilized detection probes of known identity can be used for massively parallel gene expression and gene discovery studies. A variety of detection means for measuring hybridization of tags to probes are known in the art, including fluorescent, colorimetric, radiometric, electrical, or electrochemical means.

A further object of the present invention provides a “universal chip” where the term “universal chip” refers generally to a support having arrays of detection probes selected as described above, wherein the detection probes are coupled to a detection means and further wherein hybridization of tags to probes can be detected. In a preferred embodiment, a detection means utilizes electrochemical detection of hybridization of tags to detection probes immobilized to a “universal chip” in a known array. Because the sequence of each detection probe at each location in such an array is known, the sequence of the complementary identifier tag hybridizing to a detection probe is automatically known and thus, the presence of the target corresponding to that tag is known.

Diverse methods of making oligonucleotide arrays are known, for example as disclosed in U.S. Pat. Nos. 5,412,087, 5,143,854, or 5,384,261 (the entire contents of each of which are hereby expressly incorporated by reference in their entirety) and accordingly no attempt is made to describe or catalogue all known methods. One object of the present invention provides a universal detector having detection probes attached to a support that functions as an electrical contact surface or electrode to detect hybridization of tags to detection probes. Methods for attaching oligonucleotides to an electrical contact surface are well known, for example as disclosed in any of U.S. Pat. Nos. 5,312,527, 5,776,672, 5,972,692, 6,200,761, or 6,221,586, the entire contents of each of which are hereby expressly incorporated by reference.

In the fabrication process, many other alternative materials and processes can be used. The substrate may be glass or other ceramic material; the bottom silicon dioxide can be replaced by silicon nitride, silicon dioxide deposited by other means, or other polymer materials; the conducting layer can be any appropriate material such as platinum, palladium, rhodium, a carbon composition, an oxide, or a semiconductor. For amperometric measurement either a three-electrode system consisting working electrode, counter electrode and reference electrode or a two-electrode system consisting working and a counter/reference electrode is necessary to facilitate the measurement. The working electrodes should provide a consistent surface, reproducible response from the redox species of interest, and a low background current over the potential range required for the measurement. The working electrodes may be any suitable conductive materials, preferably noble metals such as gold and platinum, or conductive carbon materials in various forms including graphite, glassy carbon and carbon paste. For a three electrode system the reference electrode is usually silver or silver/silver chloride, and the counter electrode may be prepared by any suitable materials such as noble metals, other metals such as copper and zinc, metal oxides or carbon compositions. Alternatively, the conducting layer can be prepared by screen printing of the electrode materials onto the substrate. Screen printing typically involves preparation of an organic slurry or inorganic slurry of an electrode material, such as a fine powder of carbon or gold, onto the substrate through a silk screen. The electrode material slurry may be fixed on the surface by heating or by air drying. The electrode may be any suitable conductive material such as gold, carbon, platinum, palladium, indium-tin-oxide. It is often advantageous to coat the electrode surface with a material such as avidin, streptavidin, neutravidin, or other polymers, to increase the immobilization of detection probes. Methods for the attachment include passive adsorption and covalent attachment.

If gold is chosen for the conducting layer, the layer can be evaporated, sputtered, or electroplated. A low temperature oxide layer can be replaced by spin-on dielectric materials or other polymer materials such as polyimide, or parylene. Reagent and electrical connections can be on the same side of a chip or on adjacent sides, though the opposite side configuration is preferred. Materials, temperatures, times, and dimensions may be altered to produce detectors, preferably chips, having substantially the same properties and functionality, as will be appreciated by those of skill in the art. Materials, temperatures, times, and dimensions may be altered by one of skill in the art to produce chips having the properties desired for any particular embodiment.

In a preferred embodiment, the detection probes are immobilized on a support having an array of electrodes sandwiched between two layers of silicon dioxide insulator attached to the silicon substrate, where a supporting layer is opposite the silicon substrate and the chip is oriented such that the silicon substrate is on the top and the supporting layer is on the bottom, as disclosed in U.S. patent application Ser. No. 10/121,214, filed Apr. 10, 2002, entitled Hydrophobic Zone Device and U.S. patent application Ser. No. 10/121,240, filed Apr. 10, 2002, entitled Method for Making a Molecularly Smooth Surface, the disclosures of which are incorporated herein by reference in their entireties. Preferably, gold electrodes are used. Alternately, carbon electrodes such as graphite, glassy carbon, and carbon paste can be used. In this preferred embodiment, access to the surfaces of the working electrodes, where the detection probes are immobilized, is through windows through the silicon substrate and top layer of insulator on the top surface of the chip. Windows on the underside (etched through the supporting layer and the bottom layer of insulator) allow access to a counter (or detector) electrode and a reference electrode. For gold electrodes, the two types of electrodes in the chip are selectively interconnected by deposited gold wiring within the insulating layer or by other methods known in the art. Access to the working electrode, reference electrode, and counter electrode allows a complete circuit to be formed which will enable standard techniques in the art, such as amperometric measurements, to be performed using the chip. An electrode potential applied to the working electrode, where the electrochemically active materials are present through association with the detection probes and tag sequences, will produce current proportional to the amount of tag sequence attached to the detection probes.

B.1. Detection Components: Measuring Hybridization of Identifier Tags to Complementary Detection Probes

Another aspect of the invention provides detection components for measuring hybridization of tags to detection probes. In one embodiment, DNA hybridization is detected by an electrochemical method, which generally includes observing the redox behavior of a single-stranded DNA detection probe as compared to a double-stranded DNA. For example, a voltammetric sequence-selective sensor can be used for detecting a target nucleic acid, where a double-stranded nucleic acid is contacted to a redox-active complex for example as disclosed in U.S. Pat. No. 5,312,527, the entire contents of which are hereby incorporated by reference. The complex binds non-specifically to the double-stranded DNA, and because the complex itself is the redox-active compound that provides a voltammetric signal, the complex does not function in a catalytic manner without the addition of an enzyme. Alternately, an electrochemical assay for nucleic acids can be used, in which a competitive binding event between a ligand and an antiligand is detected electrochemically, as disclosed in U.S. Pat. No. 4,840,893, the entire contents of which are hereby incorporated by reference.

In another embodiment, RNA hybridization is detected by an electrochemical method, which generally includes observing the redox behavior of a single-stranded DNA detection probe as compared to a DNA/RNA duplex formed by hybridization of an RNA tag to a DNA detection probe.

Hybridization of tags and probes may be detected using a transition metal complex capable of oxidizing at least one oxidizable base in an oxidation-reduction reaction under conditions that cause an oxidation-reduction reaction between the transition metal complex and the oxidizable base, where the probe or the tagged molecule or both contain at least one oxidizable base. The oxidation-reduction reaction indicating hybridization is detected by measuring electron transfer from each oxidized base, as disclosed in U.S. Pat. No. 5,871,981, the entire contents of which are hereby incorporated by reference.

In a preferred embodiment, hybridization of identifier tags to DNA detection probes immobilized on gold or other electrodes may be carried out using methods disclosed by Steele et al. (1998, Anal. Chem 70:4670-4677). Preferably, multivalent ions with 2, 3, or 4 positive charges are used, which are capable of electrochemical detection by direct reaction without affecting the nucleic acid. In the preferred embodiment these ions bind electrostatically to nucleic acid phosphate irrespective of whether it is in the double-helical or single-stranded form. The presence or absence of hybridized identifier tag DNA is determined for each detection probe, based on electron transfer measurements taken at each detection probe site. The sample being interrogated may be contacted with the oligonucleotide detection probe in any suitable manner known to those skilled in the art. By way of example, a DNA sample being interrogated for the presence of target nucleotide sequences may be in solution and the oligonucleotide detection probes immobilized on a solid support, whereby the DNA sample may be contacted with the oligonucleotide detection probe by immersing the solid support having the oligonucleotide detection probes immobilized thereon in the solution containing the DNA sample. Suitable transition metal complexes that bind nucleic acid electrostatically and whose reduction or oxidation is electrochemically detectable in an appropriate voltage regime include Ru(NH₃)₆³⁺, Ru(NH₃)₅pyridine³⁺ and other transition metal complexes that can be determined by one of kill in the art.

In accordance with another aspect of the present invention, oligonucleotide detection probe sequences may be designed to be redox inactive, or to have very low redox activity, for example as disclosed in U.S. Pat. No. 5,871,918. In one embodiment, oligonucleotide probe sequences are designed so as to not contain G (guanosine) bases, permitting electrochemical detection of hybridization of tagged DNA molecules by detecting G oxidation in tagged molecules with identifier tags hybridized to their probe complements, as disclosed in U.S. Pat. No. 5,871,918. Advantageously, the use of redox-inactive probes permits a high density of probes on a universal detector without a background oxidation signal.

The occurrence of the oxidation-reduction reaction may be detected according to any suitable means known to those skilled in the art. For example, the oxidation-reduction reaction may be detected using a detection electrode to observe a change in the electronic signal which is indicative of the occurrence of the oxidation-reduction reaction. Suitable reference electrodes will also be known in the art and include, for example, silver, silver/silver chloride electrodes. The electronic signal associated with the oxidation-reduction reaction permits the determination of the presence or absence of hybridized tags by measuring the Faradaic current or total charge associated with the occurrence of the oxidation-reduction reaction. The current depends on the presence of the positively charged redox ion closely associated with the electrode, which in turn depends on the amount of nucleic acid phosphate hybridized to the electrode. The electronic signal may be characteristic of any electrochemical method, including cyclic voltammetry, normal pulse voltammetry, differential pulse voltammetry, chronoamperometry, and square-wave voltammetry. The amount of hybridized DNA is determined by subtracting the current or total charge characteristic of the probes and other molecules bound to the electrode in the starting state from the current or total charge measured after the hybridization reaction.

C. Preparation of Tagged Molecules

Another aspect of the present invention provides tagged molecules generated by the target-dependent processes described herein, where tagged molecules containing identifier tags are generated only in the presence of target. Advantageously, the tag/probe sets and universal detector of the present invention provide convenient, resource-efficient materials and methods for designing and detecting tagged molecules, while target-dependent generation of tagged molecules substantially decreases or entirely eliminates the possibility of false positive signals. In a preferred embodiment, tagged molecules containing identifier tags are generated by manipulation of a template containing target nucleotide sequence. Amplification of template containing target nucleotide sequence can generate tagged molecules containing the identifier tag(s) corresponding to the target nucleotide sequence. Target-dependent probe or primer binding can also generate tagged molecules containing the identifier tag(s) corresponding to a target.

As used herein, “template” refers to all or part of a polynucleotide containing at least one target nucleotide sequence. As described above, “target nucleotide sequence” refers to the nucleotide sequence of interest in a particular application. An “exogenous nucleotide sequence” as used herein, refers to a sequence introduced during preparation of tagged molecules. The presence an identifier tag or target nucleotide sequence (or copy, complement, or portion thereof) is specifically referred to in the present disclosure, such that “exogenous nucleotide sequence” or “additional exogenous nucleotide sequence” generally refers to nucleotide sequence not found in target nucleotide sequence and identifier tag sequence. When exogenous nucleotide sequence includes sequence(s) normally found in the sample or organism from which the sample is obtained, the exogenous nucleotide sequence will be found be in an arrangement not found in the original template from which the target nucleotide sequence was copied. Preferably, exogenous nucleotide sequence is introduced by primers or probes used in target-dependent processes involved in generating tagged molecules suitable for use in the universal tag assay.

As used herein, an “auxiliary oligonucleotide” is an oligonucleotide, preferably DNA or RNA, that can be used to create a region of double-stranded DNA or RNA, or DNA/RNA heteroduplex, by incubating a single-stranded polynucleotide with an auxiliary oligonucleotide complementary to a portion of sequence on the single-stranded polynucleotide. Auxiliary nucleotides can be used to create localized regions of double-stranded DNA, RNA, or DNA/RNA to generate a restriction digestion site that permits cleavage of the single-stranded polynucleotide, or a polymerase promoter that permits polymerase binding and copying of the single-stranded polynucleotide. Auxiliary oligonucleotides can function as polymerization primers, including for rolling circle (RC) amplification. In a preferred embodiment, auxiliary oligonucleotides are complementary to one or more portions of single-stranded amplification products containing target nucleotide sequence and identifier tag sequence, and form regions of DNA duplex that create a restriction digestion site that enables trimming of the single-stranded amplification product to generate a smaller tagged molecule. Auxiliary oligonucleotides and primers may contain chemical modifications to enable trimmed single-stranded product(s) to be separated from primers and auxiliary oligonucleotides. In a preferred embodiment, the chemical modification is an addressable ligand permitting recovery of a molecule containing the ligand. In a more preferred embodiment, the addressable ligand is a biotin residue.

In accordance with another aspect of the present invention, the template may be any polynucleotide suitable for amplification, where the template contains at least one target nucleotide sequence to be amplified. Suitable templates include DNA and RNA molecules, and may include polynucleotides having modified bases. Preferably, templates are genomic DNA molecules, cDNA molecules, PCR products, LCR products, synthetic (synthesized) DNA molecules, other forms of DNA, mRNA molecules, rRNA molecules, synthetic (synthesized) RNA molecules, or other forms of RNA. Methods disclosed herein, in particular rolling circle (RC) amplification, can be used to amplify RNA templates directly without reverse-transcribing RNA template into DNA. If necessary, single-stranded template can be obtained by denaturing double-stranded DNA to generate single-stranded template, preferably target strand containing at least one target nucleotide sequence and complementary-target strand containing at least one complement of target nucleotide sequence. The double-stranded DNA may be genomic DNA.

C.1. Amplification of Target Nucleic Acid

In some embodiments, the target nucleic acid is amplified prior to addition of the complementary nucleic acids. As discussed above, the target nucleic acid may be DNA or RNA and may be provided in forms such as genomic DNA, cDNA, and mRNA. Linear or exponential (nonlinear) modes of amplification may be used with any suitable amplification method, where choice of mode is made by one of skill in the art depending on the circumstances of a particular embodiment. Methods of amplification include, but are not limited to, use of polymerase chain reaction (PCR), rolling circle amplification, and transcription with RNA polymerase.

Amplification using Polymerase Chain Reaction (PCR)

Target nucleic acid amplification by polymerase chain reaction (PCR) uses multiple rounds of primer extension reactions in which complementary strands of a defined region of a DNA molecule are simultaneously synthesized by a thermostable DNA polymerase. During repeated rounds of primer extension reactions, the number of newly synthesized DNA strands increases exponentially such that after 20 to 30 reaction cycles, the initial template can be replicated several thousand-fold or million-fold. Methods for carrying out different types and modes of PCR are thoroughly described in the literature, for example in “PCR Primer: A Laboratory Manual” Dieffenbach and Dveksler, Eds. Cold Spring Harbor Laboratory Press, 1995, and by Mullis et al. in patents (e.g., U.S. Pat. Nos. 4,683,195, 4,683,202 and 4,800,159) and scientific publications (e.g. Mullis et al. 1987, Methods in Enzymology, 155:335-350), and in U.S. patent application Ser. No. 10/138,067, the contents of each of which are hereby incorporated by reference in their entireties.

Briefly, PCR proceeds in a series of steps as described below. In the initial step of the procedure, double-stranded template is isolated and heat, preferably between about 90° C. to about 95° C., is used to separate the double-stranded DNA into single strands (denaturation step). The initial denaturation step is omitted for single-stranded template. Cooling to about 55° C. allows primers to adhere to the target region of the template, where the primers are designed to bind to regions that flank the target nucleic acid sequence (annealing step). Thermostable DNA polymerase (e.g., Taq polymerase) and free nucleotides are added to create new DNA fragments complementary to the target region of the template via primer extension (extension step), to complete one cycle of PCR. This process of denaturation, annealing and extension is repeated numerous times, preferably in a thermocycler. At the end of each cycle, each newly synthesized DNA molecule acts as a template for the next cycle, resulting in the accumulation of many hundreds or thousands, or even millions, of double-stranded amplification products from each template molecule.

In multiplex PCR, the assay is modified to include multiple primer pairs specific for distinct target nucleic acids, to allow simultaneous amplification of multiple distinct target nucleic acids and generation of multiple distinct single-stranded DNA molecules having the desired nucleotide sequence and length. For example, multiplex PCR can be carried out using the genomic DNA of an organism or an individual, where multiplex PCR will produce multiple distinct single-stranded DNA molecules.

PCR generates double-stranded amplification products suitable for post-amplification processing. PCR amplification products may contain features such as additional nucleotide sequences not found in the target nucleotide sequence. For example, in some embodiments, primers used to amplify the target nucleic acid may be designed to introduce features into amplification products by introducing exogenous nucleotide sequence(s) not found in the target nucleic acid. Such features include, but are not limited to, identifier tags, restriction digestion sites, modified nucleotides, promoter sequences, inverted repeats, chemical modifications, addressable ligands, and other non-template 5′ extensions that allow post amplification manipulation of amplification products without a significant effect on the amplification itself. Preferably, the exogenous sequences are 5′ (“upstream”) of the primer sequence involved in binding to the target nucleotide sequence. In one embodiment, primers introduce identifier tags. In another embodiment, primers introduce sites involved in restriction enzyme recognition, binding and cleavage (“trimming”) of amplification products.

If desired, one strand of the double-stranded PCR product may be removed after the amplification reaction. Several methods for removing one strand of the amplification products are available. For example, in some embodiments, the methods described in U.S. patent application Ser. No. 10/138,067, filed May 1, 2002 and entitled Amplification of DNA to Produce Single Stranded Products of Defined Sequence and Length, the disclosure of which is incorporated herein by reference in its entirety, may be used to generate single stranded PCR products. In some embodiments, one strand of the PCR product is protected from exonuclease digestion by a phosphorothioate at the 5′ terminus. In other embodiments, one strand of the PCR product comprises a biotinylated nucleotide at the 5′ terminus such that it can be removed by binding to streptavidin beads. In some embodiments, the double stranded amplification product is converted into a single stranded nucleic acid which is trimmed as described in U.S. patent application Ser. No. 10/138,067, filed May 1, 2002 and entitled Amplification of DNA to Produce Single Stranded Products of Defined Sequence and Length, the disclosure of which is incorporated herein by reference in its entirety.

Circularization of Complementary Nucleic Acids

In some embodiments, the first complementary nucleic acid and the second complementary nucleic acid are present on the same nucleic acid molecule such that ligation of the first complementary nucleic acid to the second complementary nucleic acid produces a circular nucleic acid. In such embodiments, the first complementary nucleic acid and the second complementary nucleic acid may be present at the termini of the nucleic acid molecule in which they are present. There are many variations of procedures which utilize a circular ligation product, including those specifically enumerated in Table 1 and discussed below.

Sequestering agents which interact with one or both of the complementary nucleic acids are utilized to reduce the likelihood that the complementary nucleic acids will hybridize to non-target nucleic acids in the sample. If the target nucleic acid is present in the sample, a circular ligation product will be produced.

In some embodiments, after circularization of the nucleic acid containing the first complementary nucleic acid and the second complementary nucleic acid, an exonuclease is added to remove any non-circularized molecules.

In some embodiments, after exonuclease treatment, the circularized molecule or a portion thereof may be amplified using procedures such as PCR to generate an amplification product which indicates that a ligation product containing the first complementary nucleic acid and the second complementary nucleic acid was formed (thereby also indicating the presence of the target nucleic acid in the sample). The probes used in the PCR reaction may be outside of the first complementary nucleic acid and the second complementary nucleic acid such that the PCR product contains the full sequences of the first complementary nucleic acid and the second complementary nucleic acid. Alternatively, one or both of the probes used in PCR reaction may be within the first complementary nucleic acid and the second complementary nucleic acid such that the PCR product contains a portion of one or both of the first complementary nucleic acid and the second complementary nucleic acid.

In some embodiments, after exonuclease treatment, the circular product is cleaved by adding an oligonucleotide complementary to a restriction digestion site contained in the circular molecule and digesting with the corresponding enzyme.

In some embodiments, the nucleic acid molecule comprising the first complementary nucleic acid and the second complementary nucleic acid also has a promoter thereon. The promoter may be located at any position along the molecule. Preferably, the promoter is a promoter which may readily be used in in vitro transcription reactions. For example, the promoter may facilitate in vitro transcription using T7 RNA polymerase, R4 RNA polymerase, T3 RNA polymerase, E. coli RNA polymerase, SP6 RNA polymerase, RNA polymerase II and III, or closely homologous mutants. RNA may be transcribed from the circular or ligation product or from a linearized ligation product obtained by cleaving the circular product as described above. RNA transcribed from the promoter may be detected as described below to indicate the presence of the target nucleic acid in the sample.

In other embodiments, the promoter may not be present in the nucleic acid molecule comprising the first complementary nucleic acid and the second complementary nucleic acid but may be introduced in a subsequent manipulation which is dependent on the previous formation of a nucleic acid comprising the first complementary nucleic acid and the second complementary nucleic acid. For example, the nucleic acid comprising the first complementary nucleic acid and the second complementary nucleic acid may be used as a primer in an extension reaction, such as a rolling circle amplification reaction, PCR reaction, or other extension reaction, which generates a product containing the promoter.

The RNA or DNA products resulting from the above procedures are hybridized to an electrode on an array which is in communication with a nucleotide sequence complementary to at least a portion of the RNA or DNA products such that hybridization of the RNA or DNA products with the complementary nucleotide sequences on the array indicates the presence of the target nucleic acid in the sample. In some embodiments, the electrodes may be in communication with a nucleotide sequence complementary to a tag sequence in the RNA or DNA products. In other embodiments, the electrodes may be in communication with a nucleotide sequence which is complementary to at least a portion of the target nucleic acid or a sequence complementary to at least a portion of the target nucleic acid.

Use of Ligation Products as Primers for Rolling Circle Amplification

In one embodiment, each complementary nucleic acid containing sequence complementary to a portion of the target nucleotide sequence also contains exogenous nucleotide sequence complementary to a portion of the backbone of an RC padlock probe that contains a copy of the target nucleotide sequence and a complement of the identifier tag sequence for that target nucleotide sequence. If the target nucleotide sequence is present in the sample, the complementary nucleic acids will preferentially hybridize to the target nucleotide sequence relative to the sequestering agent(s) and a ligation product will be formed. The ligation product formed by these complementary nucleic acids includes sequence complementary to the target nucleotide sequence flanked by 5′ and 3′ exogenous nucleotide sequence complementary to a portion of the backbone of the RC padlock probe. The ligation product is then incubated with at least one linear RC padlock probe, under conditions that promote hybridization of the linear RC padlock probe to the ligation product, such that the 5′ end of the linear RC padlock probe is adjacent to the 3′ end of the linear RC padlock probe and the 5′ and 3′ ends are ligated to form a circularized RC padlock probe. DNA polymerase is added to the complex formed by the circularized RC padlock probe and the ligation product, under conditions that permit RC amplification of the RC padlock probe using the ligation product as a polymerization primer.

In this embodiment, the amplification product is a single-stranded DNA molecule containing multiple repeating copies of the RC probe, including but not limited to copies of the complement of the target nucleotide sequence, copies of the identifier tag sequence, and copies of any additional sequence found in the RC probe. This amplification product is a tagged molecule suitable for use in the universal tag assay. The amplification product may include modified nucleotides, addressable ligands, sites for enzymatic digestion, or other modifications. In another embodiment, the amplification product additionally contains exogenous nucleotide sequence involved in post-amplification trimming of the amplification product to yield smaller tagged molecules suitable for use in the universal tag assay. In accordance with this aspect of the invention, no additional polymerization primer is needed because the ligation product is completely complementary to the RC padlock probe and thus serves as a polymerization primer for RC amplification when DNA polymerase is added to the reaction mixture.

In another embodiment, a circular RC probe having a copy of target nucleotide sequence and a complement of the identifier tag for that target sequence is incubated with the ligation product containing sequence complementary to the target nucleotide sequence flanked by 5′ and 3′ exogenous nucleotide sequence complementary to a portion of the backbone of an RC padlock probe. The ligation product hybridizes to the complementary region of the circular RC probe and serves as an polymerization primer for RC amplification when DNA polymerase is added to the reaction mixture. Amplification of the RC probe generates tagged molecules suitable for use in the universal tag assay as described above.

Use of Amplification Products to Generate Tagged Molecules

In accordance with another aspect of the present invention, amplification products are used in further amplification steps to generate tagged molecules suitable for use in the universal tag assay. Target nucleotide sequence is amplified using PCR or LCR to generate a first amplification product suitable for use as a template. If necessary, double-stranded amplification product is denatured to generate single-stranded template. A single-stranded first amplification product containing a complement of target nucleotide sequence is incubated with the first and second complementary nucleic acids and at least one sequestering agent which specifically interacts with one of the complementary nucleic acids. A ligation product comprising the first and second complementary nucleic acids will be formed if the target nucleotide sequence is present in the sample.

A linear RC probe containing a nucleotide sequences complementary to the first and second complementary nucleic acids and a complement of the identifier tag for that target nucleotide sequence is placed in contact with the ligation product. The linear RC probe hybridizes to the ligation product and the 3′ and 5′ ends of the linear RC probe are ligated to form a circularized RC probe. RC amplification produces a second amplification product containing multiple repeating copies of the RC probe. These second amplification products are tagged molecules suitable for use in the universal tag assay. Optionally, second amplification products are trimmed to generate smaller tagged molecules suitable for use in the universal tag assay. Tagged molecules generated by this method are incubated with a universal detector and hybridization of identifier tags to complementary detection probes is measured.

In one embodiment, PCR generates double-stranded linear DNA molecules containing a copy of the target nucleotide sequence. One terminus of the PCR product contains an addressable ligand such as biotin, introduced by primers used for PCR. The linear amplification product is denatured to generate single-stranded PCR products, wherein at least one strand contains an addressable ligand at one terminus. In a preferred embodiment, a biotinylated single-stranded PCR product having a copy of the target nucleotide sequence is incubated with streptavidin-coated beads, under conditions such that the biotinylated PCR product is attached to a bead, forming a bead-target sequence complex. The bead-target sequence complex or target sequence that has been dissociated from the bead is incubated with the first and second complementary nucleic acids and the sequestering agent(s). If the target nucleotide sequence is present in the sample, a ligation product will be formed.

If desired, the bead-target sequence/ligation product duplex is denatured and the bead-target sequence is removed using streptavidin coated beads. A linear RC padlock probe that contains sequence complementary to the first and second complementary nucleic acids at its 3′ and 5′ ends, a complement of the identifier tag for that target nucleotide sequence, and additional RC probe sequence as needed or desired. The RC linear probes hybridize to the ligation product as described above, such that the 3′ and 5′ ends are adjacent and can be ligated as described herein, forming a circularized RC padlock probe. RC amplification of the RC padlock probe generates amplification molecules having repeating copies of target nucleotide sequence and identifier tag. Optionally, an additional primer may be added as a polymerization primer for RC amplification of the padlock probe. These RC amplification products are tagged molecules suitable for use in the universal tag assay, or may be trimmed to generate smaller tagged molecules suitable for use in the universal tag assay. Advantageously, RC amplification of the padlock probe may be carried out at 65° C., preferably using Bst DNA polymerase (New England Biolands, Beverly Mass.). Alternately, RC transcription of the circularized RC padlock probe using RNA polymerase produces tagged RNA molecules suitable for use in the universal tag assay.

C.2 Trimming DNA Amplification Products to Generate Tagged Molecules

One aspect of the invention provides that exogenous nucleotide sequences introduced during an amplification step may include sequences involved in trimming the amplification product to produce smaller tagged molecules suitable for use in the universal tag assay. Trimming of amplification products to produce smaller tagged molecules is not required to practice the present invention, and one of skill in the art can determine when a trimming step may be desirable. Methods and compositions for trimming amplification products are disclosed in U.S. patent application Ser. No. 10/138,067 and U.S. Provisional Patent Application No. 60/404,195, the entire contents of which are hereby incorporated by reference. In one embodiment, the exogenous nucleotide sequence may contain self-complementary sequences that form hairpin structures. These self-complementary sequences that form hairpin structures may contain at least one restriction enzyme recognition site for a restriction enzyme involved in the trimming step, and suitable restriction enzymes include Type II restriction enzymes such as EcoRI, or Type IIS restriction enzymes such as FokI. In another embodiment, the exogenous nucleotide sequence may include sequences involved in trimming the amplification product by restriction enzymes, where the exogenous sequence encodes one strand of the restriction enzyme recognition site, and the double-stranded restriction enzyme recognition site is formed upon addition of at least one auxiliary oligonucleotide. Suitable restriction enzymes include Type II restriction enzymes such as EcoRI, or Type IIS restriction enzymes such as FokI.

Amplification products may be trimmed to form at least two types of tagged molecules. In one preferred embodiment, a tagged molecule generated by trimming is a tagged target molecule that includes a copy or complement of a target, and a distinct identifier tag. In another preferred embodiment, a tagged molecule generated by trimming includes an identifier tag and no copy or complement of the target. Tagged molecules may contain additional sequences, labels, chemical modifications, and other features selected by one of skill in the art for a particular embodiment. Tagged molecules interact with the universal detector, and the identifier tag in the tagged molecule hybridizes to complementary detection probes on the universal detector. It is the identifier tag that contains information content sufficient to indicate the presence of its corresponding target in a sample.

In a preferred embodiment, double-stranded amplification products can be trimmed, generating tagged molecules that can hybridize to a universal detector. Preferably, a nicking endonuclease is used to cut at sites flanking the tag sequence, where the nicking endonuclease cleaves only one strand of DNA of a double-stranded DNA substrate. The endonuclease recognition sequences are arranged in a dyad symmetric way around the tag sequence. For example, for N.BstNB I, the recognition sequence GAGTC is placed four nucleotides 5′ (upstream) of the beginning of the tag sequence on each strand. Such a trimming operation will release a tagged molecule containing an identifier tag, where the tagged molecule has “sticky ends” generated by one or more nicking endonucleases. Nicking endonucleases suitable for use in this embodiment include but are not limited to N.Bst NBI, N.Bbv CIA, N.Alw I, N.Bbv CIB (New England Biolabs, Beverly, Mass.). Advantageously, tagged molecules with sticky ends can hybridize to a surface, preferably the universal detector, more preferably an electrode surface. Tagged molecules, preferably hybridized to a surface, can undergo linear polymerization, providing a satisfactory level of hybridization to a surface, where any linear polymers that form by polymerization are also hybridized to a surface. In a preferred embodiment, oligonucleotides immobilized on a surface are biotinylated at their 3′ end, as the sticky ends for DNA cut with N.Bst NBI and N.Alw I have 5′ overhangs. Other enzymes such as N.Bbv CIB generate sticky ends with 3′ overhangs, although the sequence need to create a nick cleavage site is more restricted than N.Bst NBI and N.Alw. Advantageously, surface hybridization of tagged molecules with sticky end enhanced by using detection probes that form a hairpin helix with biotin in the loop, providing a helix end for the tagged molecule to stack upon.

C.3. Rolling Circle Transcription to Generate Tagged RNA Molecules; Trimming RNA Molecules

Tagged RNA products suitable for use in the universal tag assay of the present invention can be generated by transcription of RC probes using methods known in the art, for example as disclosed by Kool (U.S. Pat. Nos. 6,096,880 and 6,368,802) hereby incorporated by reference. RNA synthesis by transcription of a RC probe (DNA) does not require a polymerization primer, although one may be used if desired. RNA synthesis by transcription of an RC probe does not require an RNA polymerase promoter sequence in the probe, although a RNA polymerase promoter sequence can be incorporated into the RC probe if desired. If an RC probe has no RNA polymerase promoter, transcription can be initiated at any location on the RC probe. If an RC probe has an RNA polymerase promoter, initiation of transcription is determined by the location of the promoter. Suitable RNA polymerases include but are not limited to T7, R4, T3, E. coli RNA polymerase, SP6 RNA polymerase, RNA polymerase II and III, or closely homologous mutants.

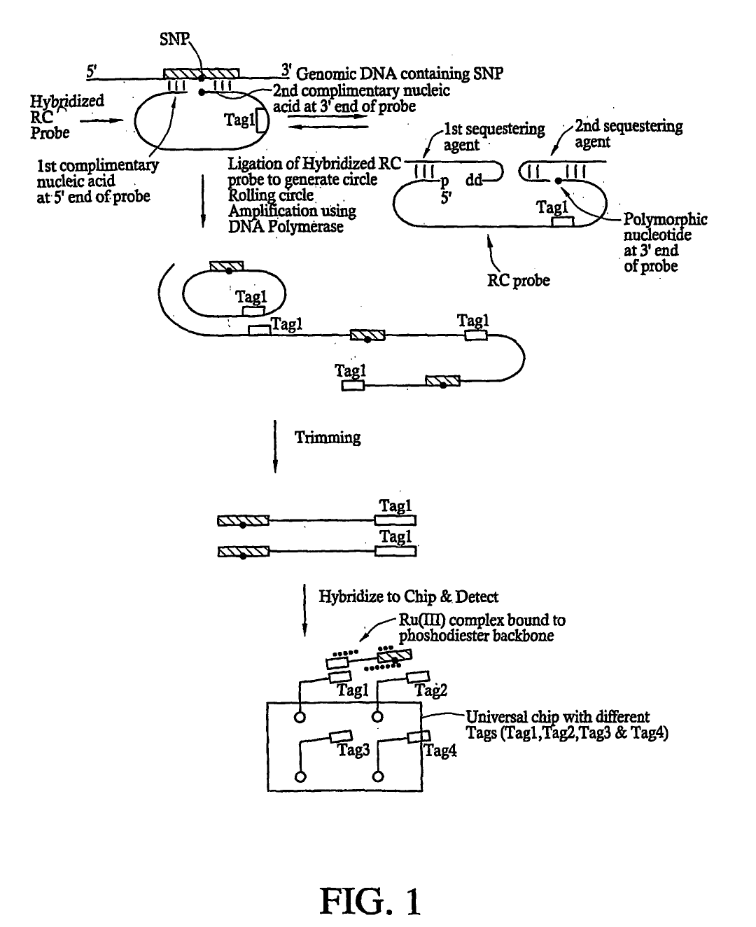

One embodiment of this method is illustrated in FIG. 1. In this aspect of the present invention provides an RC probe constructed such that it not only contains the first and second complementary nucleic acids and a complement of an identifier tag sequence for that target nucleotide sequence, but also contains a sequence that encodes at least one biologically active RNA sequence, preferably a catalytic RNA sequence. In one embodiment, the RNA product generated by RC transcription preferably encodes a ribozyme and its cleavage site. Ribozymes suitable for use with the present invention include but are not limited to hairpin ribozymes, hammerhead-motif ribozymes, and hepatitis delta catalytic RNAs.

Hammerhead-motif catalytic RNAs can readily be adapted to cleave varied RNA sequences (Uhlenbeck, 1987, Nature 328:596-600; Haseloff et al., 1988, Nature 344:585-591; Symons, 1992, Ann Rev Biochem 61:641-671; Long et al., 1993, FASEB J, 7:25-30, which references are hereby incorporated in their entirety) by altering the sequence of the noncatalytic, substrate-binding domain of the RNA encoded by the RC probe that serves as a circular DNA template. Such modifications to the sequence of the substrate-binding domain are easily made during synthesis of the RC probe, thereby permitting the method of the invention to produce any desired diagnostically or biologically useful RNA. Monomeric catalytic RNAs can act not only in cis fashion (intramolecularly) but also in trans to cleave other target RNAs (Reddy et al., U.S. Pat. No. 5,246,921; Cech et al., U.S. Pat. Nos. 4,987,071, 5,354,855, 5,093,246, the entire contents of each of which are hereby incorporated in their entirety). Catalytic RNAs produced by the invention include RNAs possessing any desired enzymatic activity, including but not limited to endo- or exo-nuclease activity, polymerase activity, ligase activity, or phosphorylase/dephosphorylase activity.

In some embodiments, the methods of the present invention produce multiple copies of a short, sequence-defined RNA oligonucleotide (oligoribonucleotide) tagged molecules formed by cleavage of the RNA product of RC transcription, where the RNA product contains repeating unit copies of the RC probe. In one embodiment, one autolytic site is present in the RC probe, such that cleavage of the transcript generates tagged RNA molecules containing target sequence and an identifier tag. In another embodiment, more than one autolytic site is present in the RC probe and the identifier tag sequence is flanked by autolytic sites, such that cleavage of the transcript generates tagged RNA molecules containing identifier tag without target sequence. In a preferred embodiment, cleavage is autolytic, as where the monomeric units contain self-cleaving ribozymes.

During transcription, the repeating RNAs may self-cleave, producing tagged molecules of monomer length, (i.e., they are cleaved to produce oligonucleotides containing only one copy of the desired sequence) after a sufficient length of time has elapsed. The monomer may contain a copy or complement of the target nucleotide sequence and the identifier tag for that target, or a monomer may contain the identifier tag without target nucleotide sequence. Typically the monomers are linear, but they may be cyclic, for example when the monomer contains a hairpin-type ribozyme capable of intramolecular ligation. The resulting monomeric tagged molecules may include catalytically active ribozymes which can sequence-specifically cleave RNA targets in trans. As an example, a self-cleaving multimer would result from inclusion of the hammerhead sequence (Forster et al., Cold Spring Harbor Symp Quant Biol, 52, 249 (1987)) in the RNA oligomer.

Cleavage of a concatemeric RNA product can also be accomplished chemically or enzymatically, as by contact with a second molecule possessing site-specific endonuclease enzymatic activity. The second molecule can be, for example, a protein or a ribozyme acting in trans to cleave a site located on a different nucleic acid. For example, an RNA multimer could also be cleaved at any sequence by using a hammerhead sequence used in trans. (Haseloff et al., 1988, Nature, 334:585). Another example of cleavage of an RNA multimer would be specific cleavage between G and A in the sequence 5 ′-GAAA, which can be achieved by the addition of the oligomer 5′-UUU and Mn²⁺, following the method of Altman disclosed by Kazakov et al. (1992, Proc Natl Acad Sci USA, 89:7939-7943), which is incorporated herein by reference. RNA can also be cleaved using catalysts such as those disclosed by Chin (1992, J Am Chem Soc 114:9792), incorporated herein by reference, which have been attached to a DNA oligomer for sequence specificity. Alternatively, the enzyme RNase H can be used with addition of a DNA oligomer, or base-specific RNases can be used.

In another embodiment, self-cleaving monomeric ribozymes produced by RC transcription of circular DNA templates (RC probes) carry “stringency clamps” that may serve to increase their substrate sequence specificity, as disclosed by Kool et al. (U.S. Pat. Nos. 6,096,880 and 6,368,802) hereby incorporated by reference. The cleavage site in the concatemeric transcript is formed by intramolecular hybridization. Self-cleavage typically results in a monomeric tagged molecule in which the 5′ and 3′ ends are folded back onto the chain and duplexed in a hairpin configuration. To cleave in cis, binding of the substrate-binding sequences of the ribozyme monomer to the substrate must successfully compete with an intramolecular complement of the substrate-binding sequences. Stringency clamps advantageously reduce the susceptibility of the tagged molecules to degradation by various agents present in media, serum and the like.

In the embodiment illustrated in FIG. 1, genomic DNA containing a SNP is obtained. A rolling circle probe comprising nucleotide the first complementary nucleic acid at its 5′ terminus and the second complementary nucleic acid at its 3′ terminus is provided. The second complementary nucleic acid contains the polymorphic nucleotide at its terminus such that it is fully complementary to one of the alleles of the SNP. The rolling circle probe also contains a nucleotide sequence complementary to a tag to be used to indicate the presence of the selected allele of the SNP in the sample as well as a sequence complementary to an autolytic RNA. First and second sequestering agents are provided such that a duplex between the rolling circle probe and the target nucleic acid is in equilibrium with duplexes between the first and second sequestering agents and the rolling circle probe. Since duplexes between the rolling circle probe and the target nucleic acid are thermodynamically favored, such duplexes are present in substantial excess over duplexes between the rolling circle probe and the first and second sequestering agents. However, the formation of duplexes between the rolling circle probe and non-target nucleic acids is significantly reduced because any first or second complementary nucleic acid sequences which are not paired with the target nucleotide sequence are paired with the sequestering agent and are not available for pairing with non-target sequences in the sample. Thus, if the sample contains the allele of the SNP complementary to the rolling circle probe, the rolling circle probe will be ligated to generate a circular molecule. It will be appreciated that although ligation of the rolling circle probe to generate a circular molecule is depicted in FIG. 1 in a step separate from hybridization of the rolling circle probe to the genomic DNA or target nucleic acid amplified therefrom, the circular molecule may also be generated at the same time that the rolling circle probe is hybridizing to the genomic DNA or the target nucleic acid generated therefrom if ligase is provided in the mixture comprising the rolling circle probe and first and second complementary nucleic acids.

A primer complementary to a portion of the rolling circle probe and RNA polymerase are provided, along with ribonucleotides. The primer is extended around the circularized molecule, generating a transcript containing repeating units of the tag and the autolytic sequence. The autolytic sequence cleaves the repeating units to generate molecules which each contain one copy of the tag (“trimmed molecules”). The trimmed molecules are placed in contact with a universal chip containing sequences complementary to the tag. Hybridization is detected by detecting Ru(III) complexes bound to the phosphodiester backbone of the hybridized nucleic acid.

Although not illustrated in FIG. 1, it will be appreciated that, if desired, two rolling circle probes each containing a second complementary nucleic acid fully complementary to one of the two alleles of the SNP and containing sequences complementary to different tags may be mixed together and placed in contact with the genomic DNA. In such embodiments, three sequestering agents may be utilized. For example, in one embodiment, the first sequestering agent is complementary to the 5′ terminal regions of both of the rolling circle probes (both rolling circle probes have the same sequence near their 5′ ends). The second sequestering agent contains a sequence complementary to one allele of the SNP while the third sequestering agent contains a sequence complementary to another allele of the SNP. The three sequestering agents and both of the rolling circle probes are placed in contact with the genomic DNA. Rolling circle probes complementary to the alleles of the SNP present in the sample are circularized and amplified as described above. The amplification products are trimmed and detected as described above. In this way, both alleles of the SNP may be assayed using a single mixture.

In some embodiments, both alleles of a large number of SNPs are simultaneously assayed in a single mixture by using rolling circle probes comprising second complementary nucleic acids which are complementary to each allele of each SNP and appropriate sequestering agents as described above. In such multiplexed reactions, each rolling circle probe contains a sequence complementary to a unique tag indicative of the presence of a particular allele of a SNP in the sample.

C.4. Generation of Tagged Molecules Following Ligation of Complementary Nucleic Acids on Separate Nucleic Acid Molecules.

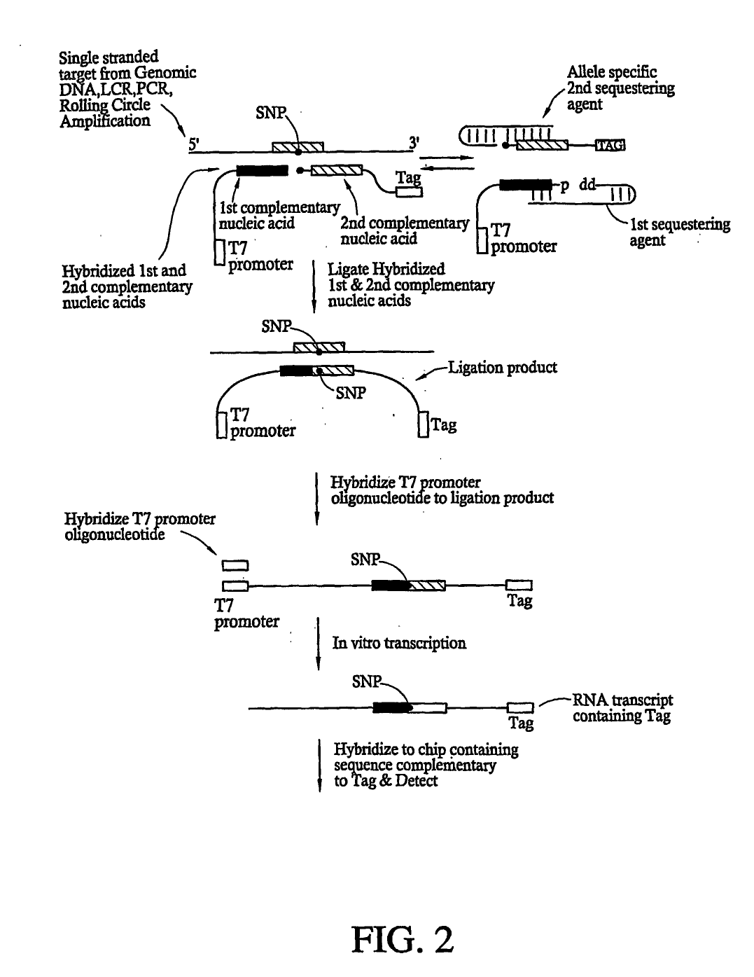

Another aspect of the present invention provides tagged molecules generated using target-dependent processes that do not involve RC amplification, as illustrated in FIG. 2. In some embodiments, the first complementary nucleic acid and the second complementary nucleic acid may be on separate molecules rather than in a single molecule. In some aspects of this embodiment, the first complementary nucleic acid may comprise a promoter from which an RNA polymerase can initiate transcription and the second complementary nucleic acid may comprise a nucleic acid complementary to the tag to be used to detect the presence of the target nucleotide sequence. At least one sequestering agent complementary to a portion of at least one of the complementary nucleic acids is provided. In some embodiments, two sequestering agents, each complementary to a portion of one of the complementary nucleic acids are provided such that both complementary nucleic acids can form complexes with the sequestering agents. If the target nucleotide sequence is present in the sample, the first and second complementary nucleic acids will preferentially hybridize to the target relative to the sequestering agent(s) and a ligation product will be generated. A tagged RNA molecule is generated by transcription of the DNA ligation product having sequence complementary to target nucleotide sequence flanked by sequence comprising a promoter from which an RNA polymerase can initiate transcription at the 3′ end of the ligation product and sequence encoding an identifier tag at the 5′ end of the ligation product. A tagged RNA molecule containing the identifier tag for a target nucleotide sequence will only be generated if both complementary nucleic acids successfully hybridized to the template target strand and were ligated. In some embodiments, target-dependent generation of tagged molecules involves the following steps: a) if desired, obtaining single-stranded template having at least one target nucleotide sequence; b) providing a first complementary nucleic acid which is complementary to a first nucleotide sequence in the target nucleic acid and a second complementary nucleic acid which is complementary to a second nucleotide sequence in the target nucleic acid c) providing one or more sequestering agents which specifically interact with either of or both the first and second complementary nucleic acids and reduce the likelihood that either of or both of the first and second complementary nucleic acids will hybridize to non-target nucleic acids d) where the first and second complementary nucleic acids are designed to hybridize to the target nucleotide sequence on the template, such that the 5′ end of one of the complementary nucleic acids hybridizes adjacent to the 3′ end of the other of the complementary nucleic acids, and the complementary nucleic acid having its 5′ end hybridized to a portion of target nucleotide sequence additionally has sequence comprising an RNA polymerase promoter at its 3′ end (or has a sequence comprising an RNA polymerase promoter positioned 3′ of the sequence complementary to the target nucleic acid), and the complementary nucleic acid having its 3′ end hybridized to a portion of target nucleotide sequence additionally has sequence complementary to an identifier tag for that target nucleotide sequence at its 5′ end (or has a sequence complementary to an identifier for that target nucleotide sequence positioned 5′ of the sequence complementary to the target nucleic acid); c) incubating the complementary nucleic acids and template under conditions that promote hybridization to template and ligation of the complementary nucleic acids to form a ligation product; d) dissociating the ligation product from the template; e) repeating the hybridization and ligation steps as desired; f) recovering ligation products and incubating with RNA polymerase and auxiliary oligonucleotide to form RNA polymerase promoter. If desired, tagged RNA molecules generated by transcription may be recovered and then used in the universal tag assay. Optionally, tagged RNA molecules generated by transcription may be recovered and purified, and then used in the universal tag assay Alternately, the transcription reaction mixture may be utilized in the universal tag assay without any intervening recovery or clean-up steps.

Although not illustrated in FIG. 2, it will be appreciated that, if desired, complementary nucleic acids complementary to both alleles of the SNP and containing sequences complementary to different tags may be mixed together and placed in contact with the genomic DNA along with first complementary nucleic acids complementary to sequences 5′ of the SNPs. In such embodiments, three complementary nucleic acids and two sequestering agents may be utilized. For example, in one embodiment, the first sequestering agent is complementary to a portion of the first complementary nucleic acid for a given SNP (the same first complementary nucleic acid may be used for both alleles of the SNP since the sequence 5′ of the SNP is not variable). The second complementary nucleic acid comprises a nucleotide sequence containing one allele of the polymorphic nucleotide of the SNP at its 3′ terminus and the second sequestering agent contains a nucleotide sequence complementary to a portion of the second complementary nucleic acid. The third complementary nucleic acid comprises a nucleotide sequence containing the other allele of the polymorphic nucleotide of the SNP at its 3′ terminus and can also form duplexes with the second sequestering agent. The three complementary nucleic acids and the two sequestering agents are placed in contact with the genomic DNA or amplified target nucleic acid obtained therefrom. An oligonucleotide comprising a T7 promoter is hybridized to the ligation products and in vitro transcription is performed to generate transcription products containing tags indicative of the allele(s) of the SNP which are present in the sample. The transcription products are hybridized to chips containing sequences complementary to the tags and detected.

In some embodiments, both alleles of a large number of SNPs are simultaneously assayed in a single mixture by using complementary nucleic acids complementary to each allele of each SNP and appropriate sequestering agents as described above.

If desired, complements of the complementary nucleic acids used in this embodiment may be designed to help prevent spurious signals. The complement of the tag-containing complementary nucleic acid may be truncated such that it does not contain the complement of the tag sequence, and may be 3′-blocked to prevent primer extension that might generate spurious copies of the tag.

This method may be used to identify variant or polymorphic sequences in a sample, including SNPs, splice variants, allelic form and mutants. Accordingly, a sample is incubated with a set of complementary nucleic acids having sequences complementary to variant sequences of the target nucleotide sequence, under conditions suitable for hybridization and ligation, wherein only those complementary nucleic acids complementary to the variant sequence present in the template will hybridize to the template and form at least one ligation product that further includes an RNA polymerase promoter and an identifier tag. In the embodiment wherein the identifier for the variant sequence is found at the 5′ end of the ligation product, the complementary nucleic acid whose 3′ end hybridizes to a portion of target nucleotide sequence may be designed to discriminate which variant sequence is present. For example, if the variant is a SNP, the nucleotide at the 3′ terminus of the complementary nucleic acid whose 3′ end hybridizes to a portion of target nucleotide sequence may be the nucleotide that discriminates which nucleotide is present in the variant being assayed. A plurality of targets, including a plurality of variant sequences, can be assayed simultaneously, as each target or variant has a distinct identifier tag that will only be present in a tagged molecule and bind to a complementary detection probe if the corresponding target was present in the sample being assayed.

Another aspect of the present invention provides a circularizable ligation product that can be used to generate an RC probe for rolling circle (RC) transcription. RC transcription generates tagged RNA molecules containing multiple repeating copies of the ligation product including the identifier tag. Tagged RNA molecules containing multiple repeating copies of sequence are suitable for use in the universal tag assay; alternately, these tagged molecules may be trimmed to generate smaller tagged RNA molecules containing an identifier tag. RC transcription advantageously provides a convenient method for generating tagged RNA molecules in any quantity desired.

If desired, PCR can be used to amplify a region surrounding a target nucleotide sequence, where PCR products may provide a suitable substrate for the ligation and transcription reactions described above.

D. Detection of Variant Sequences

In some embodiments, ligation reactions are used to identify variant or polymorphic sequences of the target nucleotide sequence present in a sample. Preferably, the variant sequence is a single nucleotide polymorphism SNP. Alternately, the variant sequence represents mutant or allelic forms of a target nucleotide sequence. In one embodiment, the amplification step is carried out using a plurality of linear RC probes, with each RC comprising a complementary nucleic acid including the polymorphic nucleotide of one of the alleles of the variants to be analyzed, such that the presence of each allele of the desired variant or polymorphic sequences may be evaluated in the sample. In some embodiments, each RC probe contains the complement of an identifier tag to be used to detect the presence of a particular variant sequence in the sample.

A sample is incubated with this plurality of linear RC probes under conditions suitable for hybridization and ligation of RC probes, such that only those RC probes or complementary nucleic acids complementary to the variant sequence(s) present in the sample will hybridize to the variant sequence(s) in a target-dependent manner and be ligated to form a circularized RC probe suitable for RC amplification, RC transcription, or other means of generating tagged molecules suitable for use in the universal tag assay.

A plurality of variant sequences of the same or different target nucleotide sequences may be detected in a single reaction using a plurality of linear RC probes as described above, wherein each linear RC probe includes sequence complementary to a single variant sequence and a complement of the identifier tag for that variant sequence. Any variant sequence that is recognized by its corresponding RC probe and then amplified or transcribed will be identified by its distinct identifier tag using the universal tag assay of the present invention.

Alternately, ligation reactions in a pre-amplification step are used to identify variant or polymorphic sequences of the target nucleotide sequence present in a sample. In such embodiments, rather than using RC probes which contain both the first and second complementary nucleic acids, the first and second complementary nucleic acids are on separate molecules, and a plurality of sets of complementary nucleic acids are used, with each set comprising a second complementary nucleic acid including the polymorphic nucleotide of one of the alleles of the variants to be analyzed, such that the presence of each allele of the desired variant or polymorphic sequences may be evaluated in the sample. In some embodiments, each second complementary nucleic acid contains the complement of an identifier tag to be used to detect the presence of a particular variant sequence in the sample.

A sample is incubated with a set of complementary nucleic acids having sequences complementary to variant sequences under conditions suitable for hybridization and ligation, wherein only those complementary nucleic acids complementary to variant sequences present in the target strand template will hybridize to the template and form at least one ligation product having sequence complementary to the variant target nucleotide sequence present in the template. In another preferred embodiment, the set of complementary nucleic acids includes exogenous sequence such that the ligation product complementary to the variant sequence further includes exogenous nucleotide sequence at its 3′ and 5′ ends that is complementary to backbone sequence flanking a copy of target nucleotide sequence in an RC probe. The ligation products can be mixed with linear RC probes for target-dependent binding and ligation of the probes. Alternately, the ligation probes can be mixed with circular RC probes. The ligation products bound to RC probes can serve as polymerization primers for amplification of RC probes having complementary variant sequence.

A plurality of variant sequences of the same or different target nucleotide sequences may be detected in a single reaction using a plurality of complementary nucleic acids as described above, wherein complementary nucleic acids including the polymorphic nucleotides of each variant sequence will produce a ligation product complementary to that variant sequence. Ligation products having sequence complementary to variant sequences present in the sample may be amplified using RC probes having complementary variant sequence and a complement of the distinct identifier tag for that variant sequence, generating tagged molecules suitable for using in the universal tag assay.

In accordance with one aspect of the present invention, ligation reactions employing sequestering agents are used to analyze variant or polymorphic sequences in the target nucleotide sequence, where such variant sequences include alleles of a locus, splice variants, or single nucleotide polymorphisms (SNPs). Advantageously, the high degree of specificity of ligation reactions permits discrimination among variant sequences to generate distinct tagged molecules corresponding to each distinct variant sequence, where the tagged molecules are easily detected using the universal tag assay.

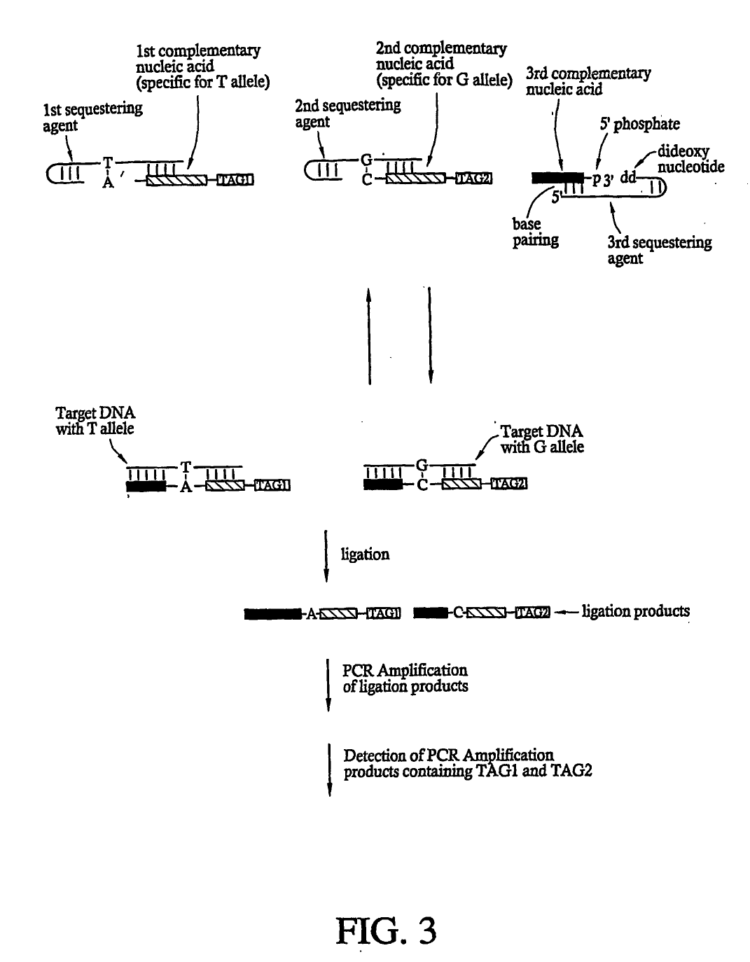

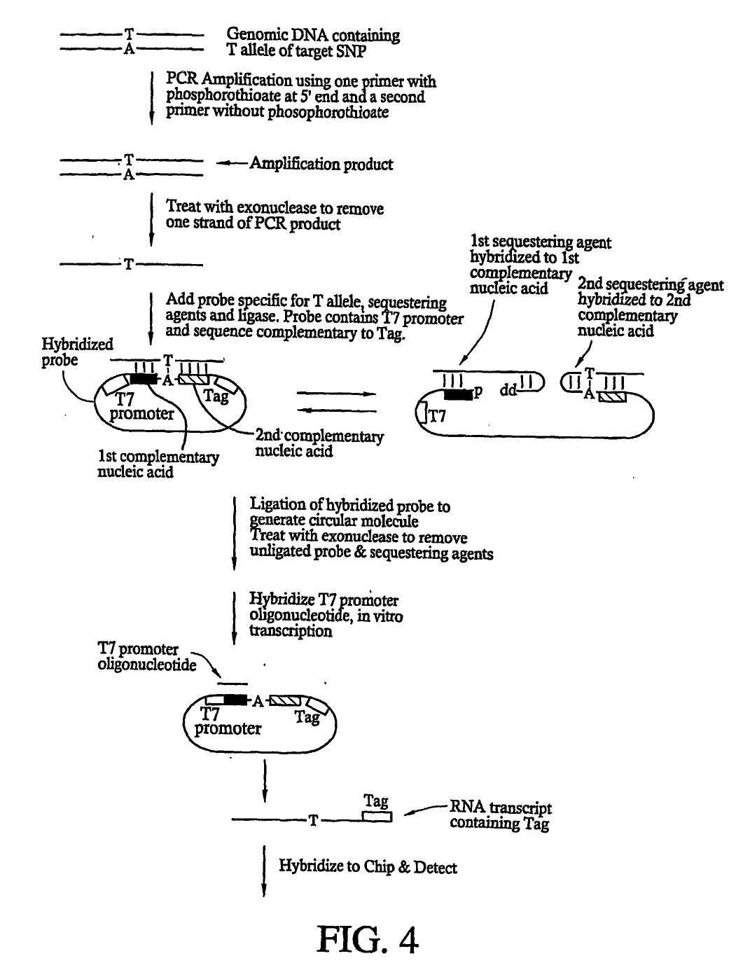

In accordance with one aspect of the present invention, a sample containing the SNP to be analyzed is contacted with complementary nucleic acids which are capable of distinguishing the different alleles of the SNP. In one embodiment, illustrated in FIG. 3, three complementary nucleic acids are placed in contact with the sample DNA, which, in this example, contains both the G allele and the T allele of a SNP. The first complementary nucleic acid is fully complementary to one of the alleles of the SNP (in FIG. 3, this complementary nucleic acid is fully complementary to the T allele of the SNP and includes an A complementary to this allele of the polymorphic nucleotide at its 3′ end), the second complementary nucleic acid is filly complementary to the G allele of the SNP and includes a C complementary to this allele of the polymorphic nucleotide at its 3′ end). The third complementary nucleic acid is complementary to the nucleotide sequence immediately 5′ of the polymorphic base in the target DNA and hybridizes to the target DNA immediately adjacent to the sequences on the target DNA to which the first and second complementary nucleic acids hybridize. A first sequestering agent comprising a nucleic acid complementary to a portion of the first complementary nucleic acid, including the polymorphic nucleotide, is provided and duplexes between the first sequestering agent and the first complementary nucleic acid are in equilibrium with duplexes between the first complementary nucleic acid and the target DNA. A second sequestering agent comprising a nucleic acid complementary to a portion of the second complementary nucleic acid, including the polymorphic nucleotide, is provided and duplexes between the second sequestering agent and the second complementary nucleic acid are in equilibrium with duplexes between the second complementary nucleic acid and the target DNA. Since duplexes between the first and second complementary nucleic acids and the target DNA are thermodynamically favored, these duplexes are present at a substantially greater concentration than duplexes between the first and second complementary nucleic acids and the first and second sequestering agents respectively. As shown in FIG. 3, a third sequestering agent comprising a nucleic acid complementary to a portion of the third complementary nucleic acid is also provided. The third sequestering agent comprises a dideoxynucleotide at its 3′ end to prevent the 5′ phosphate of the third complementary nucleic acid from being ligated to the 3′ end of the third sequestering agent. Duplexes between the third sequestering agent and the third complementary nucleic acid are in equilibrium with duplexes between the third complementary nucleic acid and the target DNA. Since duplexes between the third complementary nucleic acid and the target DNA are thermodynamically favored, these duplexes are present at a substantially greater concentration than duplexes between the third complementary nucleic acid and the third sequestering agent.

The first complementary nucleic acid hybridized to the T allele of the SNP in the sample DNA is ligated to the third complementary nucleic acid which is hybridized immediately adjacent to it, thereby generating a first ligation product. The second complementary nucleic acid hybridized to the G allele of the SNP in the sample DNA is ligated to the third complementary nucleic acid which is hybridized immediately adjacent to it, thereby generating a second ligation product.

The first and second ligation products are amplified by PCR and placed in contact with detection probes specific to the amplification products from the first or second ligation products. The ligation products are hybridized to a chip capable of detecting the presence of the first and/or second ligation products. For example, in one embodiment, the chip contains a first detection probe comprising a sequence fully complementary to at least a portion of the first ligation product which includes the polymorphic nucleotide and a second detection probe comprising a sequence fully complementary to at least a portion of the second ligation product which includes the polymorphic nucleotide. Hybridization is performed under conditions in which the signals from hybridization of the first or second ligation products to the first or second detection probes on the chip can be distinguished. Thus, the first ligation product will not contain any mismatches with the first detection probe while the second ligation product will contain a mismatch with the first detection probe. Likewise, the second ligation product will not contain any mismatches with the second detection probe while the first ligation product will contain a mismatch with the second detection probe. The signal from a completely matched ligation product may be distinguished from the signal from a ligation product containing a mismatch using techniques such as those disclosed in U.S. patent application Ser. No. 10/424,542, filed Apr. 24, 2003, entitled Universal Tag Assay, U.S. patent application Ser. No. 10/429,291, filed May 2, 2003, entitled Electrochemical Method to Measure DNA Attachment to an Electrode Surface in the Presence of Molecular Oxygen, U.S. patent application Ser. No. 10/429,293, filed May 2, 2003, entitled Method of Electrochemical Detection of Somatic Cell Mutations, or U.S. Pat. No. 6,221,586, the disclosures of which are incorporated herein by reference in their entireties. In some embodiments, hybridization may be conducted under conditions where duplexes containing a single mismatch do not form or form less efficiently so that one can determine whether the sample contains one or both alleles of the SNP by measuring the intensity of the signal at the location of the first detection probe (fully complementary to at least portion of the first ligation product including the polymorphic nucleotide) and the intensity of the signal at the location of the second detection probe (fully complementary to at least portion of the second ligation product including the polymorphic nucleotide).

E. Identification of Organisms

Another aspect of the present invention is directed to methods for identifying an organism or individual by detecting one or more target nucleotide sequences chosen to serve as distinguishing features for the organism or individual. Each target nucleotide sequence is detected using any of the assays described herein, including assays which employ but are not limited to the following steps: a) obtaining a sample having at least one target nucleotide sequence; b) generating a ligation product indicative of the presence of the target nucleotide sequence in the sample c) incubating the ligation product or a molecule generated therefrom with a universal detector having detection probes capable of detecting the ligation product or molecule generated therefrom coupled to a detection agent; and d) measuring hybridization of the ligation product or molecule generated therefrom to the detection probes. Hybridization of a ligation product or molecule generated therefrom to its complementary detection probe indicates that the sample being assayed contains the corresponding target nucleotide sequence chosen to serve as a distinguishing feature for the organism or individual.

In one embodiment, the target nucleotide sequence is detected using tagged molecules generated from a ligation product using a method including but not limited to the steps of: a) obtaining a sample having at least one target nucleotide sequence; b) generating a ligation product or molecule derived therefrom which is indicative of the presence of the target nucleotide sequence in the sample wherein the ligation product or molecule derived therefrom contains an identifier tag for that target nucleotide sequence c) incubating the tagged molecule or ligation product with a universal detector having detection probes complementary to at least a portion of the tagged molecule or ligation product coupled to detection agents; and d) detecting hybridization of distinct identifier tags to complementary detection probes. Optionally, in some embodiments, the tagged molecule is generated from an amplification product which contains exogenous nucleotide sequences including sequences involved in trimming of amplification products, such that amplification products can be trimmed to generate smaller tagged molecules suitable for use in the universal tag assay. In this embodiment, an organism or individual may be identified by detecting a tagged molecule that indicates that the sample being assayed contained the corresponding target nucleotide sequence chosen to serve as a distinguishing feature for the organism or individual from sample taken from the organism or individual.

It is understood that in embodiments employing identifier tags each identifier tag used in an application has a complementary detection probe in the universal detector or in embodiments employing ligation products which do not contain identifier tags, the detector comprises detection probes which are complementary to sequences in each of the ligation products or molecules generated therefrom which are indicative of the presence of the target nucleotide sequence in the sample. Thus, an organism or individual may be identified by the hybridization of an identifier tag, ligation product, or molecule generated therefrom to its complementary detection probe, which reliably indicates the presence of the corresponding target in a sample. In addition, an organism or individual may also be identified by the absence of hybridization of a distinct identifier tag, ligation product, or molecule generated therefrom to its complementary detection probe, which reliably indicates the absence of the corresponding target in a sample. Preferably, internal controls are included to increase the reliability of this method as described herein. In another embodiment, a multiplicity of individuals or organisms is identified by this method. Advantageously, the universal tag assay and any of the other methods disclosed herein can be used with any organism or individual without the need for custom design or manufacture of detectors.

Some embodiments of the present invention are summarized in Table 1 and Table 2 below and discussed in more detail in the following examples.

TABLE 1
Process options for SNP detection by circle formation and electrochemical readout

| | | | | | | | | | RNA | | | |
|---|---|---|---|---|---|---|---|---|---|---|---|---|
| | | Remove | | | | | | RNA | synthesis | | | |
| | | one | Add | | Exo- | | | synthesis | from | Hybridize | Amplify | |
| | Amplify | PCR | sequestering | Ligate | nuclease | Amplify | Cleave | from | linear | to | Chip | Ruthenium |
| Process: | gDNA | strand | agent | circle | treatment | circle | circle | circle | amplicon | electrode | NA | readout |
| 1. | PCR | + or − | + | + | + | − | − | + | − | RNA | + or − | + |
| 2. | PCR | + or − | + | + | + | − | + | − | + | RNA | + or − | + |
| 3. | PCR | + or − | + | + | + | − | + | − | − | DNA | + or − | + |
| 4. | − | − | + | + | + | PCR | − | − | + | RNA | + or − | + |
| 5. | − | − | + | + | + | PCR | − | − | − | DNA | + or − | + |

TABLE 2
Process options for SNP detection by oligomer ligation including suppressors of non-specific ligation and
electrochemical readout

| | | Remove | | | | | RNA | | | |
|---|---|---|---|---|---|---|---|---|---|---|
| | | one | Add | | Exo- | Amplify | synthesis | Hybridize | Amplify | |
| | Amplify | PCR | sequestering | Ligate | nuclease | ligated | from linear | to | Chip | Rutherium |
| Process: | gDNA | strand | agent | oligomers | treatment | product | amplicon | electrode | NA | readout |
| 1. | PGR | + or − | + | + | + | − | + | RNA | + or − | + |
| 2. | PGR | + or − | + | + | + | − | − | DNA | + or − | + |
| 3. | − | − | + | + | + | PCR | + | RNA | + or − | + |
| 4. | − | − | + | + | + | PCR | − | DNA | + or − | + |

### EXAMPLES

### Example 1

### RNA Transcription from a Circular Molecule

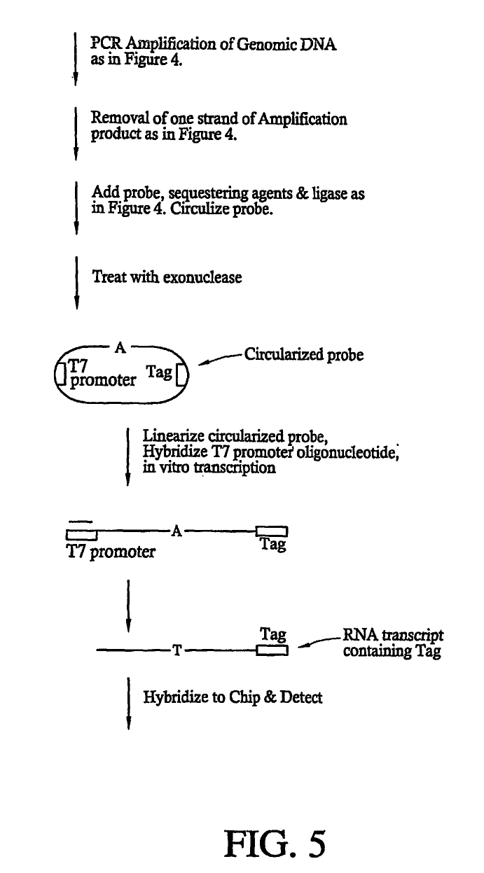

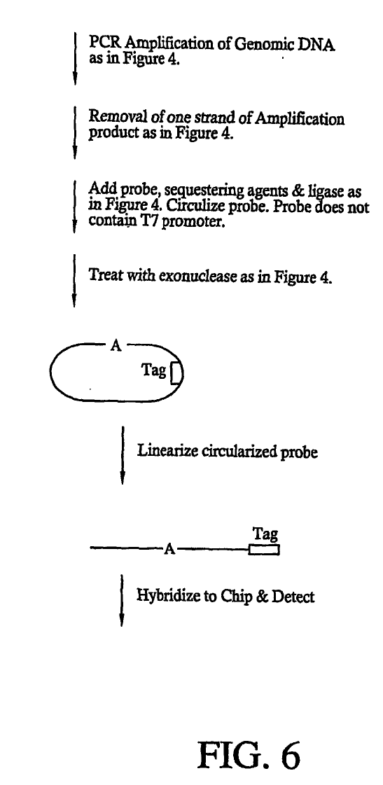

FIG. 4 and Process 1 of Table 1 summarize one embodiment of a method in which both the first complementary nucleic acid and the second complementary nucleic acid are on a single molecule.

As illustrated in FIG. 4 and summarized in Process 1 of Table 1, genomic DNA containing the T allele of a target SNP is obtained. PCR amplification is performed on the genomic DNA. If desired, as illustrated in FIG. 4 and summarized in Process 1 of Table 1, one of the primers used in the PCR reaction may have a phosphorothiote at its 5′ end in order to protect the strand of the amplification product which contains that primer from exonuclease digestion. The other primer does not contain a phosphorothioate and the strand of the amplification product which contains this primer is susceptible to exonuclease digestion.

Exonuclease digestion is performed to remove the susceptible strand of the PCR product. A nucleic acid probe comprising a first complementary nucleic acid at its 5′ end and a second complementary nucleic, acid at its 3′ end is provided. The first complementary nucleic acid is complementary to the nucleotide sequence immediately 5′ of the polymorphic nucleotide (in this case the T allele) on the remaining strand of the amplification product. The second complementary nucleic acid is complementary to the nucleotide sequence in the amplification product which includes the polymorphic nucleotide such that the A which is complementary to the T allele is at the 3′ end of the second complementary nucleic acid.

The probe also comprises a T7 promoter and a sequence complementary to a tag to be used to identify the presence of the T allele in the sample.

A first sequestering agent comprising a nucleotide sequence complementary to a portion of the first complementary nucleic acid is provided. The first sequestering agent is a DNA molecule comprising a dideoxynucleotide at its 3′ end. In addition, the region near the 3′ terminus of the first sequestering agent forms a stem loop structure. The 5′ region of the first sequestering agent is complementary to a portion of the 5′ region of the first complementary nucleic acid in the probe.

In the embodiment illustrated in FIG. 4, a second sequestering agent comprising a nucleotide sequence complementary to a portion of the second complementary nucleic acid is provided. The second sequestering agent is a DNA molecule in which the region near the 5′ terminus forms a stem loop structure. The 3′ region of the second sequestering agent is complementary to a portion of the 3′ region of the second complementary nucleic acid in the probe.

Duplexes in which the first and second complementary nucleic acids in the probe are hybridized with the strand of the amplification product containing the T allele are in equilibrium with duplexes in which the first and second complementary nucleic acids in the probe are hybridized to the first and second sequestering agents respectively. Since duplexes in which the first and second complementary nucleic acids are hybridized to the amplification product are highly thermodynamically favored relative to duplexes with the sequestering agents, most of the probe will be hybridized to the amplification product. However, because duplexes between the first and second complementary nucleic acids and the first and second sequestering agents respectively are favored over duplexes between the first and second complementary nucleic acids and non-target nucleic acids present in the sample, the level of first and second complementary nucleic acids which are bound to non-target nucleotide sequences is significantly lower than the level that would be bound in the absence of the sequestering agents. In addition, the first and second sequestering agents permit the hybridization to be conducted under non-stringent conditions.

Because the first and second complementary nucleic acids are in the terminal regions of the probe, the probe has a circular conformation when hybridized to the amplification product. When the amplification product contains the T allele complementary to the 3′ terminal nucleotide of the probe, the 5 terminus and the 3′ terminus will be ligated together to generate a circular ligation product.

An exonuclease treatment, preferably with both a 3′ and a 5′ exonuclease (but, in some embodiments only a 3′ exonuclease or only a 5′ exonuclease may be used), is used to remove unligated probe and sequestration primers. A T7 oligonucleotide is hybridized to the T7 promoter in the probe, T7 polymerase and rNTP's are added. The resulting transcript contains the tag indicative of the presence of the T allele in the sample.

The transcript is placed in contact with a detector comprising a detection probe complementary to the tag and hybridization is detected. If desired, transcripts bound to the detector may be amplified prior to detection. If desired, ruthenium amperometry may be used to detect bound nucleic acids.

It will be appreciated that, if desired, rather than utilizing both a first and second sequestering agent as described above, one may use only a single sequestering agent. If only a single sequestering agent is used, one may use either the first or second sequestering agent described above.

Although not illustrated in FIG. 4, it will be appreciated that, if desired, similar to the approach outlined above with respect to FIG. 3, two probes each having a first complementary nucleic acid at their 5′ end which is complementary to the sequence immediately adjacent to the polymorphic nucleotide and a second complementary nucleic acid at their 3′ end which includes one of the alleles of the SNP at the 3′ terminus may be mixed together and placed in contact with the amplification product generated from the genomic DNA. Each probe also includes a sequence complementary to a tag to be used to indicate the presence of the corresponding allele of the SNP in the sample. In such embodiments, three sequestering agents may be utilized as discussed above with respect to FIG. 3. The first sequestering agent is complementary to a portion of the first complementary nucleic acid, which is common to both probes. The second sequestering agent is complementary to a portion of the second complementary nucleic acid in one of the probes (including the polymorphic nucleotide of one of the alleles) while the third sequestering agent is complementary to a portion of the second complementary nucleic acid in the other probe (including the polymorphic nucleotide of the other allele). The three sequestering agents and both of the probes are placed in contact with the genomic DNA. Probes complementary to the alleles of the SNP present in the sample are circularized as described above. Transcription reactions are performed to generate transcripts comprising tags indicative of the presence of the corresponding alleles of the SNP which are present in the sample. Transcripts are detected as described above. In this way, both alleles of the SNP may be assayed using a single mixture.

In some embodiments, both alleles of a large number of SNPs are simultaneously assayed in a single mixture by using probes complementary to each allele of each SNP and appropriate sequestering agents as described above. In such multiplexed reactions, each probe contains a sequence complementary to a unique tag indicative of the presence of a particular allele of a SNP in the sample.

### Example 2

### RNA Transcription from a Linearized Circle

FIG. 5 and Process 2 of Table 1 summarize another embodiment of a method in which both the first complementary nucleic acid and the second complementary nucleic acid are on a single molecule. Genomic DNA containing the T allele of a target SNP is obtained. PCR amplification is performed on the genomic DNA. If desired, as illustrated in FIG. 4 and summarized in Process 1 of Table 1, one of the primers used in the PCR reaction may have a phosphorothiote at its 5′ end in order to protect the strand of the amplification product which contains that primer from exonuclease digestion. The other primer does not contain a phosphorothioate and the strand of the amplification product which contains this primer is susceptible to exonuclease digestion.

Exonuclease digestion is performed to remove the susceptible strand of the PCR product. A probe comprising a first complementary nucleic acid at its 5′ end and a second complementary nucleic acid at its 3′ end is provided. The first complementary nucleic acid is complementary to the nucleotide sequence immediately 5′ of the polymorphic nucleotide (in this case the T allele) on the remaining strand of the amplification product. The second complementary nucleic acid is complementary to the nucleotide sequence in the amplification product which includes the polymorphic nucleotide such that the A which is complementary to the T allele is at the 3′ end of the complementary nucleic acid.

The probe also comprises a T7 promoter and a sequence complementary to a tag to be used to identify the presence of the T allele in the sample.

A first sequestering agent comprising a nucleotide sequence complementary to a portion of the first complementary nucleic acid is provided. The first sequestering agent is a DNA molecule comprising a dideoxynucleotide at its 3′ end. In addition, the region near the 3′ terminus of the first sequestering agent forms a stem loop structure. The 5′ region of the first sequestering agent is complementary to a portion of the 5′ region of the first complementary nucleic acid in the probe.

In the embodiment illustrated in FIG. 4, a second sequestering agent comprising a nucleotide sequence complementary to a portion of the second complementary nucleic acid is provided. The second sequestering agent is a DNA molecule in which the region near the 5′ terminus forms a stem loop structure. The 3′ region of the second sequestering agent is complementary to a portion of the 3′ region of the second complementary nucleic acid in the probe.

Duplexes in which the first and second complementary nucleic acids in the probe are hybridized with the strand of the amplification product containing the T allele are in equilibrium with duplexes in which the first and second complementary nucleic acids in the probe are hybridized to the first and second sequestering agents respectively. Since duplexes in which the first and second complementary nucleic acids are hybridized to the amplification product are highly thermodynamically favored relative to duplexes with the sequestering agents, most of the probe will be hybridized to the amplification product. However, because duplexes between the first and second complementary nucleic acids and the first and second sequestering agents respectively are favored over duplexes between the first and second complementary nucleic acids and non-target nucleic acids present in the sample, the level of first and second complementary nucleic acids which are bound to non-target nucleotide sequences is significantly lower than the level that would be bound in the absence of the sequestering agents. In addition, the first and second sequestering agents permit the hybridization to be conducted under non-stringent conditions.

Because the first and second complementary nucleic acids are in the terminal regions of the probe, the probe has a circular conformation when hybridized to the amplification product. When the amplification product contains the T allele complementary to the 3′ terminal nucleotide of the probe, the 5 terminus and the 3′ terminus will be ligated together to generate a circular ligation product.

An exonuclease treatment, preferably with both a 3′ and a 5′ exonuclease (but, in some embodiments only a 3′ exonuclease or only a 5′ exonuclease may be used), is used to remove unligated probe and sequestration primers.

The circular probe is linearized by adding an oligonucleotide complementary to a restriction digestion site contained in the circular molecule and digesting with the corresponding enzyme. An oligonucleotide complementary to the T7 promoter is hybridized to the linearized probe. T7 polymerase and rNTPs are provided and in vitro transcription is performed to generate an RNA transcript comprising the tag indicative of the presence of the target allele in the sample. The transcript is placed in contact with a detector comprising a detection probe complementary to the tag and hybridization is detected as described in Example 1.

As discussed above in Example 1, it will be appreciated that, if desired, rather than utilizing both a first and second sequestering agent as described above, one may use only a single sequestering agent. If only a single sequestering agent is used, one may use either the first or second sequestering agent described above.

Although not illustrated in FIG. 5, it will be appreciated that, if desired, similar to the approach outlined above with respect to FIG. 3, two probes each having a first complementary nucleic acid at their 5′ end which is complementary to the sequence immediately adjacent to the polymorphic nucleotide and a second complementary nucleic acid at their 3′ end which includes one of the alleles of the SNP at the 3′ terminus may be mixed together and placed in contact with the amplification product generated from the genomic DNA. Each probe also includes a sequence complementary to a tag to be used to indicate the presence of the corresponding allele of the SNP in the sample. In such embodiments, three sequestering agents may be utilized as discussed above with respect to FIG. 3. The first sequestering agent is complementary to a portion of the first complementary nucleic acid, which is common to both probes. The second sequestering agent is complementary to a portion of the second complementary nucleic acid in one of the probes (including the polymorphic nucleotide of one of the alleles) while the third sequestering agent is complementary to a portion of the second complementary nucleic acid in the other probe (including the polymorphic nucleotide of the other allele). The three sequestering agents and both of the probes are placed in contact with the genomic DNA. Probes complementary to the alleles of the SNP present in the sample are circularized as described above. Transcription reactions are performed to generate transcripts comprising tags indicative of the presence of the corresponding alleles of the SNP which are present in the sample. Transcripts are detected as described above. In this way, both alleles of the SNP may be assayed using a single mixture.

In some embodiments, both alleles of a large number of SNPs are simultaneously assayed in a single mixture by using probes complementary to each allele of each SNP and appropriate sequestering agents as described above. In such multiplexed reactions, each probe contains a sequence complementary to a unique tag indicative of the presence of a particular allele of a SNP in the sample.

### Example 3

### Detection of Linearized Probe

FIG. 6 and Process 3 of Table 1 summarize another embodiment of a method in which both the first complementary nucleic acid and the second complementary nucleic acid are on a single molecule. Genomic DNA containing the T allele of a target SNP is obtained. PCR amplification is performed on the genomic DNA. If desired, as discussed above, one of the primers used in the PCR reaction may have a phosphorothiote at its 5′ end in order to protect the strand of the amplification product which contains that primer from exonuclease digestion. The other primer does not contain a phosphorothioate and the strand of the amplification product which contains this primer is susceptible to exonuclease digestion.

Exonuclease digestion is performed to remove the susceptible strand of the PCR product. A probe comprising a first complementary nucleic acid at its 5′ end and a second complementary nucleic acid at its 3′ end is provided. The first complementary nucleic acid is complementary to the nucleotide sequence immediately 5′ of the polymorphic nucleotide (in this case the T allele) on the remaining strand of the amplification product. The second complementary nucleic acid is complementary to the nucleotide sequence in the amplification product which includes the polymorphic nucleotide such that the A which is complementary to the T allele is at the 3′ end of the complementary nucleic acid.

As illustrated in FIG. 6, unlike the probe of Example 1, the probe used in the embodiment of the present Example does not comprise a T7 promoter. The probe also includes a tag which can be used to indicate the presence of the target nucleotide sequence in the sample.

A first sequestering agent comprising a nucleotide sequence complementary to a portion of the first complementary nucleic acid is provided. The first sequestering agent is a DNA molecule comprising a dideoxynucleotide at its 3′ end. In addition, the region near the 3′ terminus of the first sequestering agent forms a stem loop structure. The 5′ region of the first sequestering agent is complementary to a portion of the 5′ region of the first complementary nucleic acid in the probe.

In the embodiment illustrated in FIG. 6, a second sequestering agent comprising a nucleotide sequence complementary to a portion of the second complementary nucleic acid is provided. The second sequestering agent is a DNA molecule in which the region near the 5′ terminus forms a stem loop structure. The 3′ region of the second sequestering agent is complementary to a portion of the 3′ region of the second complementary nucleic acid in the probe.

Duplexes in which the first and second complementary nucleic acids in the probe are hybridized with the strand of the amplification product containing the T allele are in equilibrium with duplexes in which the first and second complementary nucleic acids in the probe are hybridized to the first and second sequestering agents respectively. Since duplexes in which the first and second complementary nucleic acids are hybridized to the amplification product are highly thermodynamically favored relative to duplexes with the sequestering agents, most of the probe will be hybridized to the amplification product. However, because duplexes between the first and second complementary nucleic acids and the first and second sequestering agents respectively are favored over duplexes between the first and second complementary nucleic acids and non-target nucleic acids present in the sample, the level of first and second complementary nucleic acids which are bound to non-target nucleotide sequences is significantly lower than the level that would be bound in the absence of the sequestering agents. In addition, the first and second sequestering agents permit the hybridization to be conducted under non-stringent conditions.

Because the first and second complementary nucleic acids are in the terminal regions of the probe, the probe has a circular conformation when hybridized to the amplification product. When the amplification product contains the T allele complementary to the 3′ terminal nucleotide of the probe, the 5 terminus and the 3′ terminus will be ligated together to generate a circular ligation product.

An exonuclease treatment, preferably with both a 3′ and a 5′ exonuclease (but, in some embodiments only a 3′ exonuclease or only a 5′ exonuclease may be used), is used to remove unligated probe and sequestration primers.

The circular probe is linearized by adding an oligonucleotide complementary to a restriction digestion site contained in the circular molecule and digesting with the corresponding enzyme. The linearized probe comprising a tag to be used to indicate the presence of the target allele in the sample is placed in contact with a detector comprising a detection probe complementary to the tag and hybridization is detected as described in Example 1.

As discussed above in Example 1, it will be appreciated that, if desired, rather than utilizing both a first and second sequestering agent as described above, one may use only a single sequestering agent. If only a single sequestering agent is used, one may use either the first or second sequestering agent described above.

Although not illustrated in FIG. 6, it will be appreciated that, if desired, similar to the approach outlined above with respect to FIG. 3, two probes each having a first complementary nucleic acid at their 5′ end which is complementary to the sequence immediately adjacent to the polymorphic nucleotide and a second complementary nucleic acid at their 3′ end which includes one of the alleles of the SNP at the 3′ terminus may be mixed together and placed in contact with the amplification product generated from the genomic DNA. Each probe also includes a sequence complementary to a tag to be used to indicate the presence of the corresponding allele of the SNP in the sample. In such embodiments, three sequestering agents may be utilized as discussed above with respect to FIG. 3. The first sequestering agent is complementary to a portion of the first complementary nucleic acid, which is common to both probes. The second sequestering agent is complementary to a portion of the second complementary nucleic acid in one of the probes (including the polymorphic nucleotide of one of the alleles) while the third sequestering agent is complementary to a portion of the second complementary nucleic acid in the other probe (including the polymorphic nucleotide of the other allele). The three sequestering agents and both of the probes are placed in contact with the genomic DNA. Probes complementary to the alleles of the SNP present in the sample are circularized and cleaved as described above. Linearized probes are detected as described above. In this way, both alleles of the SNP may be assayed using a single mixture.

In some embodiments, both alleles of a large number of SNPs are simultaneously assayed in a single mixture by using probes complementary to each allele of each SNP and appropriate sequestering agents as described above. In such multiplexed reactions, each probe contains a sequence complementary to a unique tag indicative of the presence of a particular allele of a SNP in the sample.

### Example 4

### Detection without Amplification of Genomic DNA

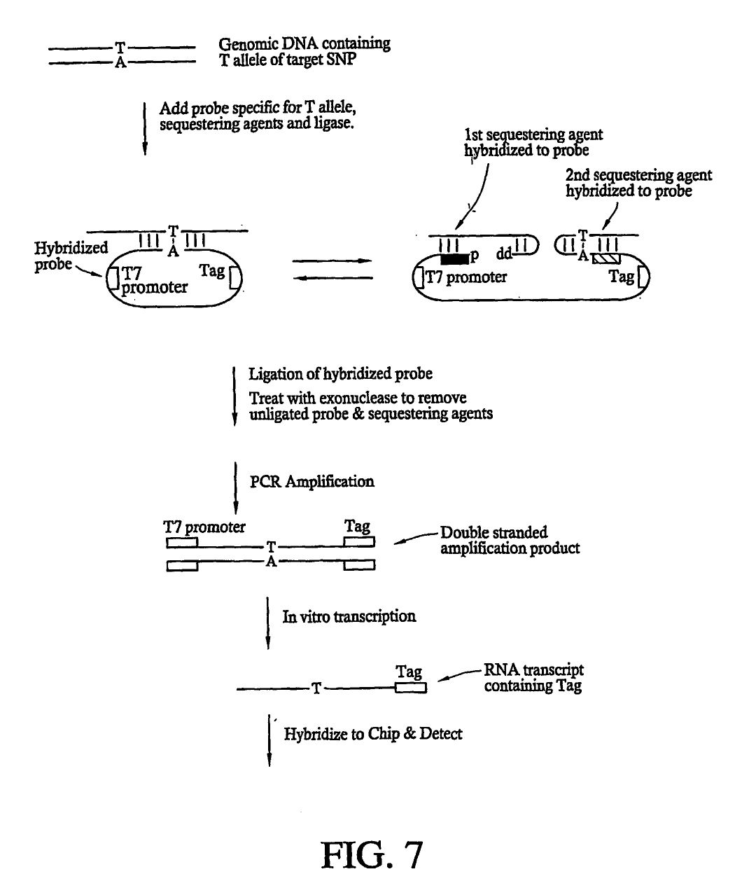

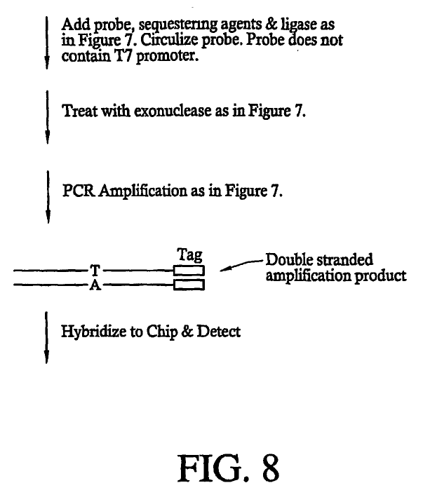

FIG. 7 and Process 4 of Table 1 summarize one embodiment of a method in which both the first complementary nucleic acid and the second complementary nucleic acid are on a single molecule.

As illustrated in FIG. 7 and summarized in Process 4 of Table 1, genomic DNA containing the T allele of a target SNP is obtained. A probe comprising a first complementary nucleic acid at its 5′ end and a second complementary nucleic acid at its 3′ end is provided. The first complementary nucleic acid is complementary to the nucleotide sequence immediately 5′ of the polymorphic nucleotide (in this case the T allele) in the genomic DNA. The second complementary nucleic acid is complementary to the nucleotide sequence in the genomic DNA which includes the polymorphic nucleotide such that the A which is complementary to the T allele is at the 3′ end of the second complementary nucleic acid.

The probe also comprises a T7 promoter and a sequence complementary to a tag which can be used to identify the presence of the T allele in the sample.

A first sequestering agent comprising a nucleotide sequence complementary to a portion of the first complementary nucleic acid is provided. The first sequestering agent is a DNA molecule comprising a dideoxynucleotide at its 3′ end. In addition, the region near the 3′ terminus of the first sequestering agent forms a stem loop structure. The 5′ region of the first sequestering agent is complementary to a portion of the 5′ region of the first complementary nucleic acid in the probe.

In the embodiment illustrated in FIG. 7, a second sequestering agent comprising a nucleotide sequence complementary to a portion of the second complementary nucleic acid is provided. The second sequestering agent is a DNA molecule in which the region near the 5′ terminus forms a stem loop structure. The 3′ region of the second sequestering agent is complementary to a portion of the 3′ region of the second complementary nucleic acid in the probe.

Duplexes in which the first and second complementary nucleic acids in the probe are hybridized with the target nucleotide sequence in the genomic DNA containing the T allele are in equilibrium with duplexes in which the first and second complementary nucleic acids in the probe are hybridized to the first and second sequestering agents respectively. Since duplexes in which the first and second complementary nucleic acids are hybridized to the target nucleotide sequence are highly thermodynamically favored relative to duplexes with the sequestering agents, most of the probe will be hybridized to the target nucleotide sequence in the genomic DNA. However, because duplexes between the first and second complementary nucleic acids and the first and second sequestering agents respectively are favored over duplexes between the first and second complementary nucleic acids and non-target nucleic acids present in the sample, the level of first and second complementary nucleic acids which are bound to non-target nucleotide sequences is significantly lower than the level that would be bound in the absence of the sequestering agents. In addition, the first and second sequestering agents permit the hybridization to be conducted under non-stringent conditions.

Because the first and second complementary nucleic acids are in the terminal regions of the probe, the probe has a circular conformation when hybridized to the target nucleotide sequence in the genomic DNA. When the genomic DNA contains the T allele complementary to the 3′ terminal nucleotide of the probe, the 5 terminus and the 3′ terminus will be ligated together to generate a circular ligation product.

An exonuclease treatment, preferably with both a 3′ and a 5′ exonuclease (but, in some embodiments only a 3′ exonuclease or only a 5′ exonuclease may be used), is used to remove unligated probe and sequestration primers. PCR is conducted on the circularized probe to generate a linear amplicon comprising the T7 promoter and the tag. A T7 oligonucleotide is hybridized to the T7 promoter in the linear amplicon and T7 polymerase and rNTP's are added. The resulting transcript contains the tag indicative of the presence of the T allele in the sample.

The transcript is placed in contact with a detector comprising a detection probe complementary to the tag and hybridization is detected. If desired, transcripts bound to the detector may be amplified prior to detection. If desired, ruthenium amperometry may be used to detect bound nucleic acids.

It will be appreciated that, if desired, rather than utilizing both a first and second sequestering agent as described above, one may use only a single sequestering agent. If only a single sequestering agent is used, one may use either the first or second sequestering agent described above.

Although not illustrated in FIG. 7, it will be appreciated that, if desired, similar to the approach outlined above with respect to FIG. 3, two probes each having a first complementary nucleic acid at their 5′ end which is complementary to the sequence immediately adjacent to the polymorphic nucleotide and a second complementary nucleic acid at their 3′ end which includes one of the alleles of the SNP at the 3′ terminus may be mixed together and placed in contact with the genomic DNA. Each probe also includes a sequence complementary to a tag to be used to indicate the presence of the corresponding allele of the SNP in the sample. In such embodiments, three sequestering agents may be utilized as discussed above with respect to FIG. 3. The first sequestering agent is complementary to a portion of the first complementary nucleic acid, which is common to both probes. The second sequestering agent is complementary to a portion of the second complementary nucleic acid in one of the probes (including the polymorphic nucleotide of one of the alleles) while the third sequestering agent is complementary to a portion of the second complementary nucleic acid in the other probe (including the polymorphic nucleotide of the other allele). The three sequestering agents and both of the probes are placed in contact with the genomic DNA. Probes complementary to the alleles of the SNP present in the sample are circularized as described above. Transcription reactions are performed to generate transcripts comprising tags indicative of the presence of the corresponding alleles of the SNP which are present in the sample. Transcripts are detected as described above. In this way, both alleles of the SNP may be assayed using a single mixture.

In some embodiments, both alleles of a large number of SNPs are simultaneously assayed in a single mixture by using probes complementary to each allele of each SNP and appropriate sequestering agents as described above. In such multiplexed reactions, each probe contains a sequence complementary to a unique tag indicative of the presence of a particular allele of a SNP in the sample.

### Example 5

### Detection of Linear Amplification Product

FIG. 8 and Process 5 of Table 1 summarize another embodiment of a method in which both the first complementary nucleic acid and the second complementary nucleic acid are on a single molecule.

As illustrated in FIG. 8 and summarized in Process 5 of Table 1, genomic DNA containing the T allele of a target SNP is obtained. A probe comprising a first complementary nucleic acid at its 5′ end and a second complementary nucleic acid at its 3′ end is provided. The first complementary nucleic acid is complementary to the nucleotide sequence immediately 5′ of the polymorphic nucleotide (in this case the T allele) in the target nucleotide sequence in the genomic DNA. The second complementary nucleic acid is complementary to the target nucleotide sequence in the genomic DNA which includes the polymorphic nucleotide such that the A which is complementary to the T allele is at the 3′ end of the complementary nucleic acid.

The probe also comprises a sequence complementary to or identical to a tag to be used to identify the presence of the T allele in the sample.

A first sequestering agent comprising a nucleotide sequence complementary to a portion of the first complementary nucleic acid is provided. The first sequestering agent is a DNA molecule comprising a dideoxynucleotide at its 3′ end. In addition, the region near the 3′ terminus of the first sequestering agent forms a stem loop structure. The 5′ region of the first sequestering agent is complementary to a portion of the 5′ region of the first complementary nucleic acid in the probe.

In the embodiment illustrated in FIG. 8, a second sequestering agent comprising a nucleotide sequence complementary to a portion of the second complementary nucleic acid is provided. The second sequestering agent is a DNA molecule in which the region near the 5′ terminus forms a stem loop structure. The 3′ region of the second sequestering agent is complementary to a portion of the 3′ region of the second complementary nucleic acid in the probe.

Duplexes in which the first and second complementary nucleic acids in the probe are hybridized with the strand of the target nucleotide sequence in the genomic DNA containing the T allele are in equilibrium with duplexes in which the first and second complementary nucleic acids in the probe are hybridized to the first and second sequestering agents respectively. Since duplexes in which the first and second complementary nucleic acids are hybridized to the target nucleotide sequence in the genomic DNA are highly thermodynamically favored relative to duplexes with the sequestering agents, most of the probe will be hybridized to the amplification product. However, because duplexes between the first and second complementary nucleic acids and the first and second sequestering agents respectively are favored over duplexes between the first and second complementary nucleic acids and non-target nucleic acids present in the sample, the level of first and second complementary nucleic acids which are bound to non-target nucleotide sequences is significantly lower than the level that would be bound in the absence of the sequestering agents. In addition, the first and second sequestering agents permit the hybridization to be conducted under non-stringent conditions.

Because the first and second complementary nucleic acids are in the terminal regions of the probe, the probe has a circular conformation when hybridized to the target nucleotide sequence. When the genomic DNA contains the T allele complementary to the 3′ terminal nucleotide of the probe, the 5 terminus and the 3′ terminus will be ligated together to generate a circular ligation product.

An exonuclease treatment, preferably with both a 3′ and a 5′ exonuclease (but, in some embodiments only a 3′ exonuclease or only a 5′ exonuclease may be used), is used to remove unligated probe and sequestration primers. PCR is conducted on the circularized probe to generate a linear amplicon comprising the tag. The linear amplicon is placed in contact with a detector comprising a detection probe complementary to the tag and hybridization is detected as described above.

It will be appreciated that, if desired, rather than utilizing both a first and second sequestering agent as described above, one may use only a single sequestering agent. If only a single sequestering agent is used, one may use either the first or second sequestering agent described above.

Although not illustrated in FIG. 8, it will be appreciated that, if desired, similar to the approach outlined above with respect to FIG. 3, two probes each having a first complementary nucleic acid at their 5′ end which is complementary to the sequence immediately adjacent to the polymorphic nucleotide and a second complementary nucleic acid at their 3′ end which includes one of the alleles of the SNP at the 3′ terminus may be mixed together and placed in contact with the genomic DNA. Each probe also includes a sequence complementary to or identical to a tag to be used to indicate the presence of the corresponding allele of the SNP in the sample. In such embodiments, three sequestering agents may be utilized as discussed above with respect to FIG. 3. The first sequestering agent is complementary to a portion of the first complementary nucleic acid, which is common to both probes. The second sequestering agent is complementary to a portion of the second complementary nucleic acid in one of the probes (including the polymorphic nucleotide of one of the alleles) while the third sequestering agent is complementary to a portion of the second complementary nucleic acid in the other probe (including the polymorphic nucleotide of the other allele). The three sequestering agents and both of the probes are placed in contact with the genomic DNA. Probes complementary to the alleles of the SNP present in the sample are circularized as described above. PCR is performed to generate amplification products and amplification products are detected as described above. In this way, both alleles of the SNP may be assayed using a single mixture.

In some embodiments, both alleles of a large number of SNPs are simultaneously assayed in a single mixture by using probes complementary to each allele of each SNP and appropriate sequestering agents as described above. In such multiplexed reactions, each probe contains a sequence complementary to a unique tag indicative of the presence of a particular allele of a SNP in the sample.

### Example 6

### SNP Detection by Oligomer Ligation

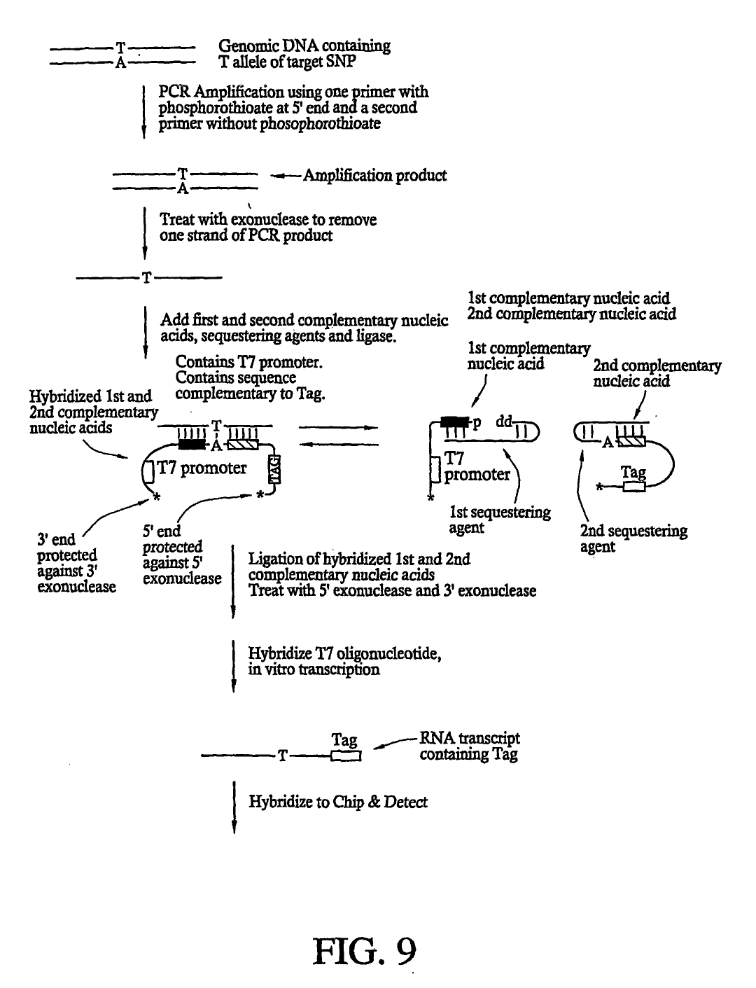

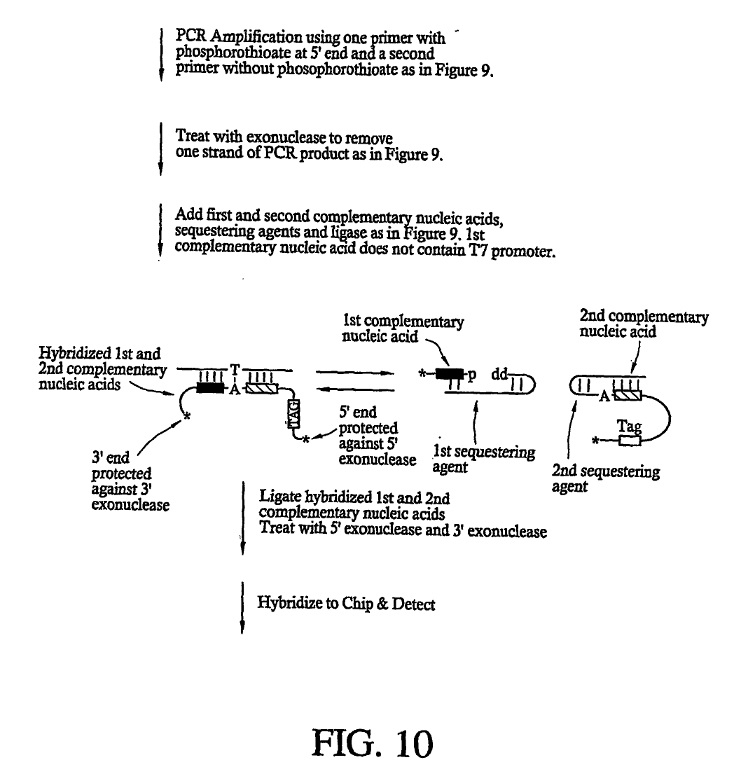

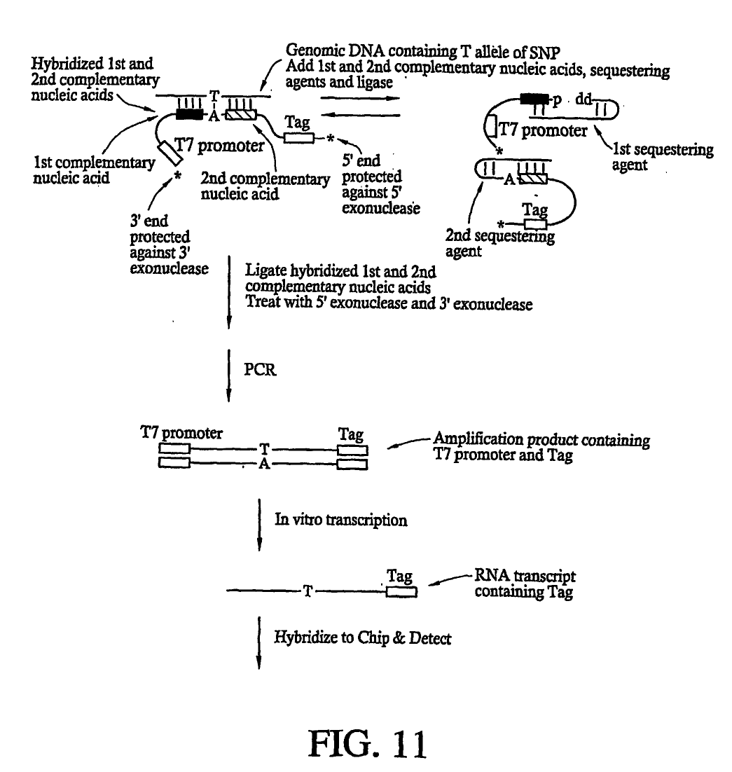

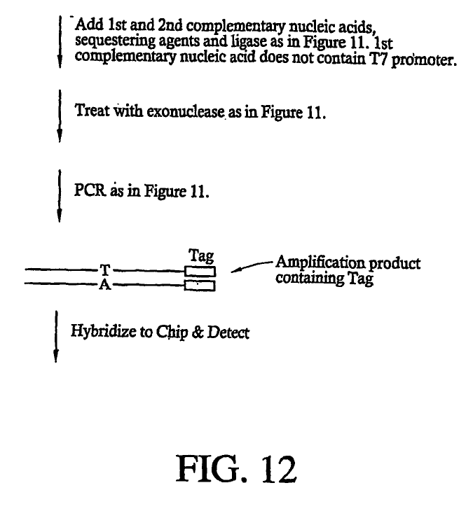

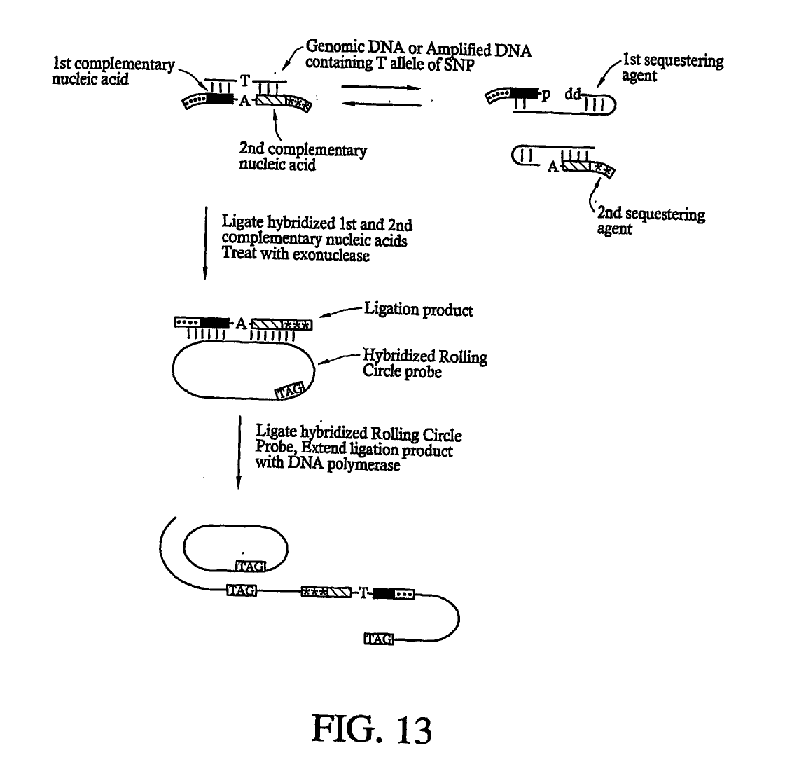

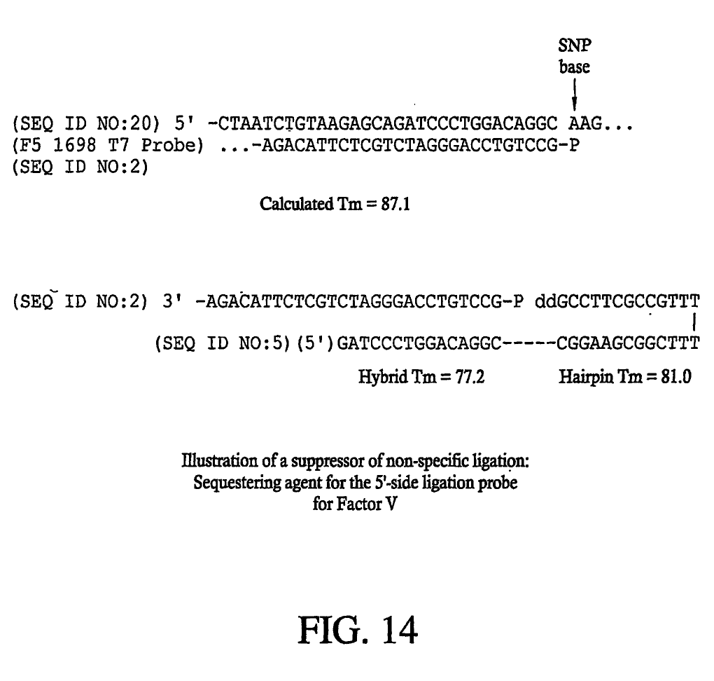

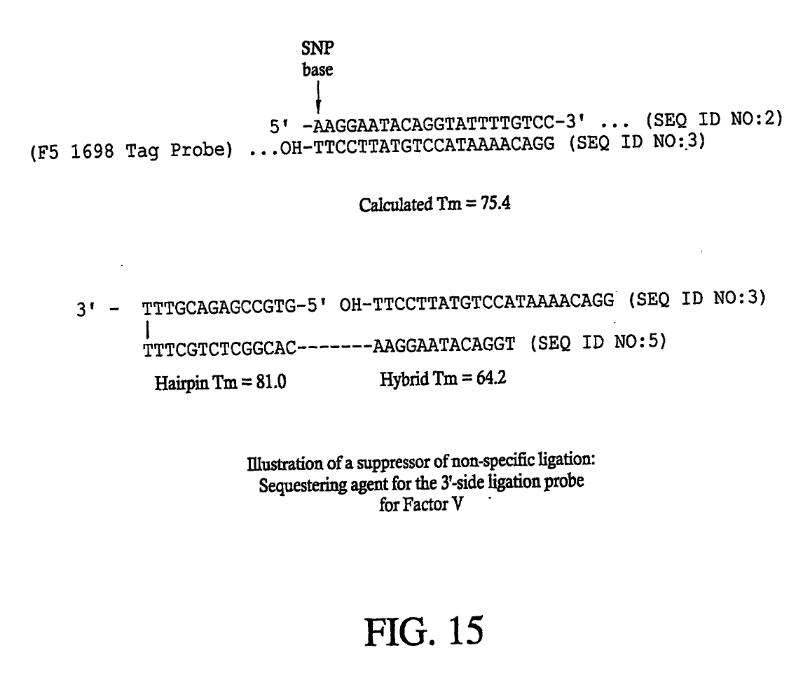

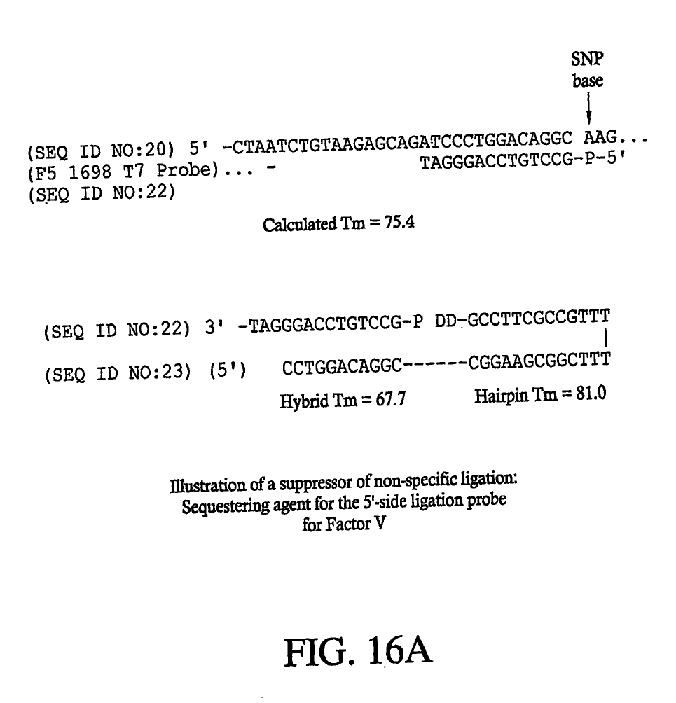

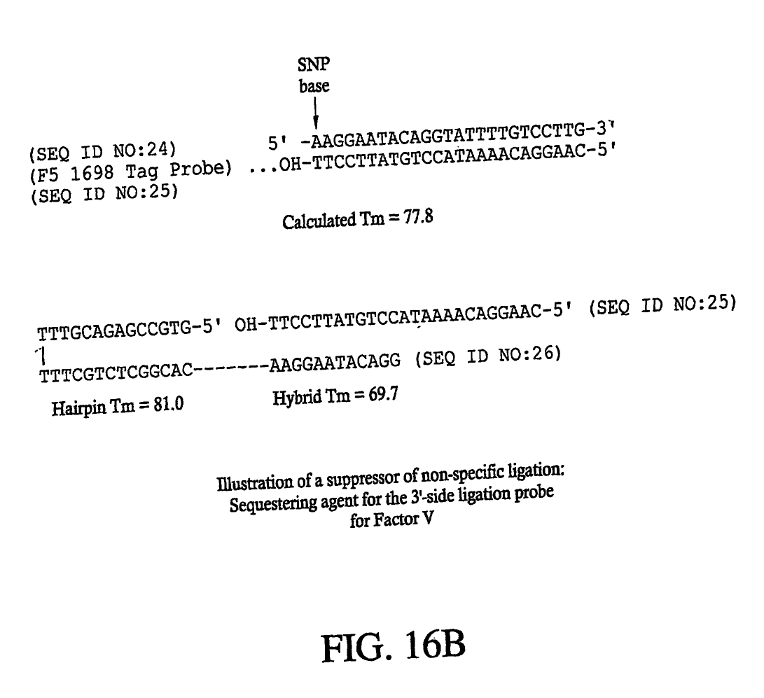

FIG. 9 and Process 1 of Table 2 summarize an embodiment of a method in which the first and second complementary nucleic acids are separate molecules. As illustrated in FIG. 9 and summarized in Process 1 of Table 2, genomic DNA containing the T allele of a target SNP is obtained. PCR amplification is performed on the genomic DNA. If desired, as discussed above, one of the primers used in the PCR reaction may have a phosphorothiote at its 5′ end in order to protect the strand of the amplification product which contains that primer from exonuclease digestion. The other primer does not contain a phosphorothioate and the strand of the amplification product which contains this primer is susceptible to exonuclease digestion.

Exonuclease digestion is performed to remove the susceptible strand of the PCR product. A first complementary nucleic acid and a second complementary nucleic acid are provided. The first complementary nucleic acid is complementary to the nucleotide sequence immediately 5′ of the polymorphic nucleotide (in this case the T allele) on the remaining strand of the amplification product. The first complementary nucleic acid also comprises a T7 promoter. The second complementary nucleic acid is complementary to the nucleotide sequence in the amplification product which includes the polymorphic nucleotide such that the A which is complementary to the T allele is at the 3′ end of the second complementary nucleic acid. The second complementary nucleic acid also comprises a nucleotide sequence complementary to a tag to be used to indicate the presence of the T allele of the SNP in the sample.

A first sequestering agent comprising a nucleotide sequence complementary to a portion of the first complementary nucleic acid is provided. The first sequestering agent is a DNA molecule comprising a dideoxynucleotide at its 3′ end. In addition, the region near the 3′ terminus of the first sequestering agent forms a stem loop structure. The 5′ region of the first sequestering agent is complementary to a portion of the 5′ region of the first complementary nucleic acid in the probe.

In the embodiment illustrated in FIG. 9, a second sequestering agent comprising a nucleotide sequence complementary to a portion of the second complementary nucleic acid is provided. The second sequestering agent is a DNA molecule in which the region near the 5′ terminus forms a stem loop structure. The 3′ region of the second sequestering agent is complementary to a portion of the 3′ region of the second complementary nucleic acid in the probe.

Duplexes in which the first and second complementary nucleic acids are hybridized with the strand of the amplification product containing the T allele are in equilibrium with duplexes in which the first and second complementary nucleic acids are hybridized to the first and second sequestering agents respectively. Since duplexes in which the first and second complementary nucleic acids are hybridized to the amplification product are highly thermodynamically favored relative to duplexes with the sequestering agents, most of the first and second complementary nucleic acids will be hybridized to the amplification product. However, because duplexes between the first and second complementary nucleic acids and the first and second sequestering agents respectively are favored over duplexes between the first and second complementary nucleic acids and non-target nucleic acids present in the sample, the level of first and second complementary nucleic acids which are bound to non-target nucleotide sequences is significantly lower than the level that would be bound in the absence of the sequestering agents. In addition, the first and second sequestering agents permit the hybridization to be conducted under non-stringent conditions.

Because the first and second complementary nucleic acids are complementary to adjacent nucleotide sequences in the amplification product, when the amplification product contains the T allele complementary to the 3′ terminal nucleotide of the second complementary nucleic acid, the first complementary nucleic acid and the second complementary nucleic acid will be ligated together to generate a ligation product.

In some embodiments, anexonuclease treatment, preferably with both a 3′ and a 5′ exonuclease (but, in some embodiments only a 3′ exonuclease or only a 5′ exonuclease may be used), is used to remove sequestering agents and unligated complementary nucleic acids. For example, in some embodiments, the first and second complementary nucleic acids may be protected from digestion by including phosphorothioate linkages at the 3′ and 5′ ends distal to the ligation site.

A T7 oligonucleotide is hybridized to the T7 promoter in the ligation product, T7 polymerase and rNTP's are added. The resulting transcript contains the tag indicative of the presence of the T allele in the sample.

The transcript is placed in contact with a detector comprising a detection probe complementary to the tag and hybridization is detected as described above.

It will be appreciated that, if desired, rather than utilizing both a first and second sequestering agent as described above, one may use only a single sequestering agent. If only a single sequestering agent is used, one may use either the first or second sequestering agent described above.

Although not illustrated in FIG. 9, it will be appreciated that, if desired, similar to the approach outlined above with respect to FIG. 3, both alleles of the SNP may be detected using a single mixture. In such embodiments, three complementary nucleic acids are used. The three complementary nucleic acids are on separate molecules. The first complementary nucleic acid is complementary to the sequence 5′ of the polymorphic nucleotide in the amplification product. The second complementary nucleic acid includes a first allele of the polymorphic nucleotide at its 3′ terminus as well as a sequence complementary to the a portion of the sequence 3′ of the polymorphic nucleotide in the amplification product. The third complementary nucleic acid includes a second allele of the polymorphic nucleotide at its 3′ terminus as well as a sequence complementary to a portion of the sequence 3′ of the polymorphic nucleotide in the amplification product. The second and third complementary nucleic acids also contain sequences complementary to different tags. The first, second and third complementary nucleic acids are mixed together and placed in contact with the amplification product.

For example, in one embodiment, the same first complementary nucleic acid may be used to detect each of the alleles of the SNP. The sequestering agents and first, second and third complementary nucleic acids are placed in contact with the amplification product. If the sample contains both alleles of the SNP, two ligation products will be generated. One ligation product comprises the first complementary nucleic acid ligated to the second complementary nucleic acid. The other ligation product comprises the first complementary nucleic acid ligated to the third complementary nucleic acid. Transcription reactions are performed to generate transcripts comprising tags indicative of the presence of the corresponding alleles of the SNP which are present in the sample. Transcripts are detected as described above. In this way, both alleles of the SNP may be assayed using a single mixture.

In some embodiments, both alleles of a large number of SNPs are simultaneously assayed in a single mixture by using complementary nucleic acids complementary to each allele of each SNP and appropriate sequestering agents as described above. In such multiplexed reactions, for each SNP the second complementary nucleic acid contains a sequence complementary to a unique tag indicative of the presence of a particular allele of a SNP in the sample.

### Example 7

### SNP Detection by Oligomer Ligation without Transcription from Ligation Product

FIG. 10 and Process 2 of Table 2 summarize another embodiment of a method in which the first and second complementary nucleic acids are separate molecules. As illustrated in FIG. 10 and summarized in Process 2 of Table 2, genomic DNA containing the T allele of a target SNP is obtained. PCR amplification is performed on the genomic DNA. If desired, as discussed above, one of the primers used in the PCR reaction may have a phosphorothiote at its 5′ end in order to protect the strand of the amplification product which contains that primer from exonuclease digestion. The other primer does not contain a phosphorothioate and the strand of the amplification product which contains this primer is susceptible to exonuclease digestion.

Exonuclease digestion is performed to remove the susceptible strand of the PCR product. A first complementary nucleic acid and a second complementary nucleic acid are provided. The first complementary nucleic acid is complementary to the nucleotide sequence immediately 5′ of the polymorphic nucleotide (in this case the T allele) on the remaining strand of the amplification product. Unlike the first complementary nucleic acid of Example 6 above, the first complementary nucleic acid used in the embodiment of the present Example does not comprise a T7 promoter. The second complementary nucleic acid is complementary to the nucleotide sequence in the amplification product which includes the polymorphic nucleotide such that the A which is complementary to the T allele is at the 3′ end of the complementary nucleic acid. The second complementary nucleic acid also comprises a tag to be used to indicate the presence of the T allele of the SNP in the sample.

A first sequestering agent comprising a nucleotide sequence complementary to a portion of the first complementary nucleic acid is provided. The first sequestering agent is a DNA molecule comprising a dideoxynucleotide at its 3′ end. In addition, the region near the 3′ terminus of the first sequestering agent forms a stem loop structure. The 5′ region of the first sequestering agent is complementary to a portion of the 5′ region of the first complementary nucleic acid in the probe.

In the embodiment illustrated in FIG. 10, a second sequestering agent comprising a nucleotide sequence complementary to a portion of the second complementary nucleic acid is provided. The second sequestering agent is a DNA molecule in which the region near the 5′ terminus forms a stem loop structure. The 3′ region of the second sequestering agent is complementary to a portion of the 3′ region of the second complementary nucleic acid in the probe.

Duplexes in which the first and second complementary nucleic acids are hybridized with the strand of the amplification product containing the T allele are in equilibrium with duplexes in which the first and second complementary nucleic acids are hybridized to the first and second sequestering agents respectively. Since duplexes in which the first and second complementary nucleic acids are hybridized to the amplification product are highly thermodynamically favored relative to duplexes with the sequestering agents, most of the first and second complementary nucleic acids will be hybridized to the amplification product. However, because duplexes between the first and second complementary nucleic acids and the first and second sequestering agents respectively are favored over duplexes between the first and second complementary nucleic acids and non-target nucleic acids present in the sample, the level of first and second complementary nucleic acids which are bound to non-target nucleotide sequences is significantly lower than the level that would be bound in the absence of the sequestering agents. In addition, the first and second sequestering agents permit the hybridization to be conducted under non-stringent conditions.

Because the first and second complementary nucleic acids are complementary to adjacent nucleotide sequences in the amplification product, when the amplification product contains the T allele complementary to the 3′ terminal nucleotide of the second complementary nucleic acid, the first complementary nucleic acid and the second complementary nucleic acid will be ligated together to generate a ligation product.

In some embodiments, an exonuclease treatment, preferably with both a 3′ and a 5′ exonuclease (but, in some embodiments only a 3′ exonuclease or only a 5′ exonuclease may be used), is used to remove sequestering agents and unligated complementary nucleic acids. For example, in some embodiments, the first and second complementary nucleic acids may be protected from digestion by including phosphorothioate linkages at the 3′ and 5′ ends distal to the ligation site. Ligation products are placed in contact with a detector comprising a detection probe complementary to the tag and hybridization is detected as described above.

It will be appreciated that, if desired, rather than utilizing both a first and second sequestering agent as described above, one may use only a single sequestering agent. If only a single sequestering agent is used, one may use either the first or second sequestering agent described above.

Although not illustrated in FIG. 10, it will be appreciated that, if desired, similar to the approach outlined above with respect to FIG. 3, both alleles of the SNP may be detected using a single mixture. In such embodiments, three complementary nucleic acids are used. The first complementary nucleic acid is complementary to the sequence 5′ of the polymorphic nucleotide in the amplification product. The second complementary nucleic acid includes a first allele of the polymorphic nucleotide at its 3′ terminus as well as a sequence complementary to the sequence 3′ of the polymorphic nucleotide in the amplification product. The third complementary nucleic acid includes a second allele of the polymorphic nucleotide at its 3′ terminus as well as a sequence complementary to the sequence 3′ of the polymorphic nucleotide in the amplification product. The second and third complementary nucleic acids also contain different tag sequences. The first, second and third complementary nucleic acids are mixed together and placed in contact with the amplification product.

For example, in one embodiment, the same first complementary nucleic acid may be used to detect each of the alleles of the SNP. In such embodiments, three sequestering agents may be utilized. The three sequestering agents and first, second and third complementary nucleic acids are placed in contact with the amplification product. If the sample contains both alleles of the SNP, two ligation products will be generated. One ligation product comprises the first complementary nucleic acid ligated to the second complementary nucleic acid. The other ligation product comprises the first complementary nucleic acid ligated to the third complementary nucleic acid. Ligation products are detected as described above. In this way, both alleles of the SNP may be assayed using a single mixture.

In some embodiments, both alleles of a large number of SNPs are simultaneously assayed in a single mixture by using complementary nucleic acids complementary to each allele of each SNP and appropriate sequestering agents as described above. In such multiplexed reactions, for each SNP the second complementary nucleic acid contains a unique tag indicative of the presence of a particular allele of a SNP in the sample.

### Example 8

### SNP Detection by Oligomer Ligation Using Amplified Ligation Product

FIG. 11 and Process 3 of Table 2 summarize another embodiment of a method in which the first and second complementary nucleic acids are separate molecules. As illustrated in FIG. 11 and summarized in Process 3 of Table 2, genomic DNA containing the T allele of a target SNP is obtained.

A first complementary nucleic acid and a second complementary nucleic acid are provided. The first complementary nucleic acid is complementary to the nucleotide sequence immediately 5′ of the polymorphic nucleotide (in this case the T allele) on the genomic DNA. The first complementary nucleic acid also comprises a T7 promoter. The second complementary nucleic acid is complementary to the nucleotide sequence in genomic DNA which includes the polymorphic nucleotide such that the A which is complementary to the T allele is at the 3′ end of the complementary nucleic acid. The second complementary nucleic acid also comprises a nucleotide sequence complementary to a tag to be used to indicate the presence of the T allele of the SNP in the sample.

A first sequestering agent comprising a nucleotide sequence complementary to a portion of the first complementary nucleic acid is provided. The first sequestering agent is a DNA molecule comprising a dideoxynucleotide at its 3′ end. In addition, the region near the 3′ terminus of the first sequestering agent forms a stem loop structure. The 5′ region of the first sequestering agent is complementary to a portion of the 5′ region of the first complementary nucleic acid in the probe.

In the embodiment illustrated in FIG. 11, a second sequestering agent comprising a nucleotide sequence complementary to a portion of the second complementary nucleic acid is provided. The second sequestering agent is a DNA molecule in which the region near the 5′ terminus forms a stem loop structure. The 3′ region of the second sequestering agent is complementary to a portion of the 3′ region of the second complementary nucleic acid in the probe.

Duplexes in which the first and second complementary nucleic acids are hybridized with the strand of the genomic DNA containing the T allele are in equilibrium with duplexes in which the first and second complementary nucleic acids are hybridized to the first and second sequestering agents respectively. Since duplexes in which the first and second complementary nucleic acids are hybridized to the genomic DNA are highly thermodynamically favored relative to duplexes with the sequestering agents, most of the first and second complementary nucleic acids will be hybridized to the genomic DNA. However, because duplexes between the first and second complementary nucleic acids and the first and second sequestering agents respectively are favored over duplexes between the first and second complementary nucleic acids and non-target nucleic acids present in the sample, the level of first and second complementary nucleic acids which are bound to non-target nucleotide sequences is significantly lower than the level that would be bound in the absence of the sequestering agents. In addition, the first and second sequestering agents permit the hybridization to be conducted under non-stringent conditions.

Because the first and second complementary nucleic acids are complementary to adjacent nucleotide sequences in the genomic DNA, when the genomic DNA contains the T allele complementary to the 3′ terminal nucleotide of the second complementary nucleic acid, the first complementary nucleic acid and the second complementary nucleic acid will be ligated together to generate a ligation product.

In some embodiments, an exonuclease treatment, preferably with both a 3′ and a 5′ exonuclease (but, in some embodiments only a 3′ exonuclease or only a 5′ exonuclease may be used), is used to remove sequestering agents and unligated complementary nucleic acids. For example, in some embodiments, the first and second complementary nucleic acids may be protected from digestion by including phosphorothioate linkages at the 3′ and 5′ ends distal to the ligation site. The ligation product is amplified using PCR. T7 polymerase and rNTP's are added. The resulting transcript contains the tag indicative of the presence of the T allele in the sample.

The transcript is placed in contact with a detector comprising a detection probe complementary to the tag and hybridization is detected as described above.

It will be appreciated that, if desired, rather than utilizing both a first and second sequestering agent as described above, one may use only a single sequestering agent. If only a single sequestering agent is used, one may use either the first or second sequestering agent described above.

Although not illustrated in FIG. 11, it will be appreciated that, if desired, similar to the approach outlined above with respect to FIG. 3, both alleles of the SNP may be detected using a single mixture. In such embodiments, three complementary nucleic acids are used. The first complementary nucleic acid is complementary to the sequence 5′ of the polymorphic nucleotide in the genomic DNA. The second complementary nucleic acid includes a first allele of the polymorphic nucleotide at its 3′ terminus as well as a sequence complementary to the sequence 3′ of the polymorphic nucleotide in the genomic DNA. The third complementary nucleic acid includes a second allele of the polymorphic nucleotide at its 3′ terminus as well as a sequence complementary to the sequence 3′ of the polymorphic nucleotide in the genomic DNA. The second and third complementary nucleic acids also contain sequences complementary to different tags. The first, second and third complementary nucleic acids are mixed together and placed in contact with the genomic DNA.

For example, in one embodiment, the same first complementary nucleic acid may be used to detect each of the alleles of the SNP. The sequestering agents and first, second and third complementary nucleic acids are placed in contact with the genomic DNA. If the sample contains both alleles of the SNP, two ligation products will be generated. One ligation product comprises the first complementary nucleic acid ligated to the second complementary nucleic acid. The other ligation product comprises the first complementary nucleic acid ligated to the third complementary nucleic acid. Transcription reactions are performed to generate transcripts comprising tags indicative of the presence of the corresponding alleles of the SNP which are present in the sample. Transcripts are detected as described above. In this way, both alleles of the SNP may be assayed using a single mixture.

In some embodiments, both alleles of a large number of SNPs are simultaneously assayed in a single mixture by using complementary nucleic acids complementary to each allele of each SNP and appropriate sequestering agents as described above. In such multiplexed reactions, for each SNP the second complementary nucleic acid contains a sequence complementary to a unique tag indicative of the presence of a particular allele of a SNP in the sample.

### Example 9

### SNP Detection by Oligomer Ligation Using Amplified Ligation Product

FIG. 12 and Process 4 of Table 2 summarize another embodiment of a method in which the first and second complementary nucleic acids are separate molecules. As illustrated in FIG. 12 and summarized in Process 4 of Table 2, genomic DNA containing the T allele of a target SNP is obtained.

A first complementary nucleic acid and a second complementary nucleic acid are provided. The first complementary nucleic acid is complementary to the nucleotide sequence immediately 5′ of the polymorphic nucleotide (in this case the T allele) on the genomic DNA. Unlike the embodiment of Example 8 above, in the embodiment of the present Example, the first complementary nucleic acid does not comprises a T7 promoter. The second complementary nucleic acid is complementary to the nucleotide sequence in the genomic DNA which includes the polymorphic nucleotide such that the A which is complementary to the T allele is at the 3′ end of the complementary nucleic acid. The second complementary nucleic acid also comprises a nucleotide sequence complementary to a tag to be used to indicate the presence of the T allele of the SNP in the sample.

A first sequestering agent comprising a nucleotide sequence complementary to a portion of the first complementary nucleic acid is provided. The first sequestering agent is a DNA molecule comprising a dideoxynucleotide at its 3′ end. In addition, the region near the 3′ terminus of the first sequestering agent forms a stem loop structure. The 5′ region of the first sequestering agent is complementary to a portion of the 5′ region of the first complementary nucleic acid in the probe.

In the embodiment illustrated in FIG. 12, a second sequestering agent comprising a nucleotide sequence complementary to a portion of the second complementary nucleic acid is provided. The second sequestering agent is a DNA molecule in which the region near the 5′ terminus forms a stem loop structure. The 3′ region of the second sequestering agent is complementary to a portion of the 3′ region of the second complementary nucleic acid in the probe.

Duplexes in which the first and second complementary nucleic acids are hybridized with the strand of the genomic DNA containing the T allele are in equilibrium with duplexes in which the first and second complementary nucleic acids are hybridized to the first and second sequestering agents respectively. Since duplexes in which the first and second complementary nucleic acids are hybridized to the genomic DNA are highly thermodynamically favored relative to duplexes with the sequestering agents, most of the first and second complementary nucleic acids will be hybridized to the genomic DNA. However, because duplexes between the first and second complementary nucleic acids and the first and second sequestering agents respectively are favored over duplexes between the first and second complementary nucleic acids and non-target nucleic acids present in the sample, the level of first and second complementary nucleic acids which are bound to non-target nucleotide sequences is significantly lower than the level that would be bound in the absence of the sequestering agents. In addition, the first and second sequestering agents permit the hybridization to be conducted under non-stringent conditions.

Because the first and second complementary nucleic acids are complementary to adjacent nucleotide sequences in the genomic DNA, when the genomic DNA contains the T allele complementary to the 3′ terminal nucleotide of the second complementary nucleic acid, the first complementary nucleic acid and the second complementary nucleic acid will be ligated together to generate a ligation product.

In some embodiments, an exonuclease treatment, preferably with both a 3′ and a 5′ exonuclease (but, in some embodiments only a 3′ exonuclease or only a 5′ exonuclease may be used), is used to remove sequestering agents and unligated complementary nucleic acids. For example, in some embodiments, the first and second complementary nucleic acids may be protected from digestion by including phosphorothioate linkages at the 3′ and 5′ ends distal to the ligation site. The ligation product is amplified using PCR. The amplification product is placed in contact with a detector comprising a detection probe complementary to the tag and hybridization is detected as described above.

It will be appreciated that, if desired, rather than utilizing both a first and second sequestering agent as described above, one may use only a single sequestering agent. If only a single sequestering agent is used, one may use either the first or second sequestering agent described above.

Although not illustrated in FIG. 12, it will be appreciated that, if desired, similar to the approach outlined above with respect to FIG. 3, both alleles of the SNP may be detected using a single mixture. In such embodiments, three complementary nucleic acids are used. The first complementary nucleic acid is complementary to the sequence 5′ of the polymorphic nucleotide in the genomic DNA. The second complementary nucleic acid includes a first allele of the polymorphic nucleotide at its 3′ terminus as well as a sequence complementary to the sequence 3′ of the polymorphic nucleotide in the genomic DNA. The third complementary nucleic acid includes a second allele of the polymorphic nucleotide at its 3′ terminus as well as a sequence complementary to the sequence 3′ of the polymorphic nucleotide in the genomic DNA. The second and third complementary nucleic acids also contain sequences complementary to different tags. The first, second and third complementary nucleic acids are mixed together and placed in contact with the genomic DNA.

For example, in one embodiment, the same first complementary nucleic acid may be used to detect each of the alleles of the SNP. The sequestering agents and first, second and third complementary nucleic acids are placed in contact with the genomic DNA. If the sample contains both alleles of the SNP, two ligation products will be generated. One ligation product comprises the first complementary nucleic acid ligated to the second complementary nucleic acid. The other ligation product comprises the first complementary nucleic acid ligated to the third complementary nucleic acid. Amplification reactions are performed to generate amplification products comprising tags indicative of the presence of the corresponding alleles of the SNP which are present in the sample. Amplification products are detected as described above. In this way, both alleles of the SNP may be assayed using a single mixture.

In some embodiments, both alleles of a large number of SNPs are simultaneously assayed in a single mixture by using complementary nucleic acids complementary to each allele of each SNP and appropriate sequestering agents as described above. In such multiplexed reactions, for each SNP the second complementary nucleic acid contains a sequence complementary to a unique tag indicative of the presence of a particular allele of a SNP in the sample.

### Example 10

### SNP Detection by Oligomer Ligation Followed By Rolling Circle Amplification

FIG. 13 summarizes another embodiment of a method in which the first and second complementary nucleic acids are separate molecules. As illustrated in FIG. 13 genomic DNA containing the T allele of a target SNP is obtained.

A first complementary nucleic acid and a second complementary nucleic acid are provided. The first complementary nucleic acid is complementary to the nucleotide sequence immediately 5′ of the polymorphic nucleotide (in this case the T allele) on the genomic DNA. The first complementary nucleic acid also includes a sequence complementary to a portion of an RC probe. The second complementary nucleic acid is complementary to the nucleotide sequence in the genomic DNA which includes the polymorphic nucleotide such that the A which is complementary to the T allele is at the 3′ end of the complementary nucleic acid. The second complementary nucleic acid also comprises a nucleotide sequence complementary to a portion of the RC probe.

A first sequestering agent comprising a nucleotide sequence complementary to a portion of the first complementary nucleic acid is provided. The first sequestering agent is a DNA molecule comprising a dideoxynucleotide at its 3′ end. In addition, the region near the 3′ terminus of the first sequestering agent forms a stem loop structure. The 5′ region of the first sequestering agent is complementary to a portion of the 5′ region of the first complementary nucleic acid in the probe.

In the embodiment illustrated in FIG. 13, a second sequestering agent comprising a nucleotide sequence complementary to a portion of the second complementary nucleic acid is provided. The second sequestering agent is a DNA molecule in which the region near the 5′ terminus forms a stem loop structure. The 3′ region of the second sequestering agent is complementary to a portion of the 3′ region of the second complementary nucleic acid in the probe.

Duplexes in which the first and second complementary nucleic acids are hybridized with the strand of the genomic DNA containing the T allele are in equilibrium with duplexes in which the first and second complementary nucleic acids are hybridized to the first and second sequestering agents respectively. Since duplexes in which the first and second complementary nucleic acids are hybridized to the genomic DNA are highly thermodynamically favored relative to duplexes with the sequestering agents, most of the first and second complementary nucleic acids will be hybridized to the genomic DNA. However, because duplexes between the first and second complementary nucleic acids and the first and second sequestering agents respectively are favored over duplexes between the first and second complementary nucleic acids and non-target nucleic acids present in the sample, the level of first and second complementary nucleic acids which are bound to non-target nucleotide sequences is significantly lower than the level that would be bound in the absence of the sequestering agents. In addition, the first and second sequestering agents permit the hybridization to be conducted under non-stringent conditions.

Because the first and second complementary nucleic acids are complementary to adjacent nucleotide sequences in the genomic DNA, when the genomic DNA contains the T allele complementary to the 3′ terminal nucleotide of the second complementary nucleic acid, the first complementary nucleic acid and the second complementary nucleic acid will be ligated together to generate a ligation product.

In some embodiments, an exonuclease treatment, preferably with both a 3′ and a 5′ exonuclease (but, in some embodiments only a 3′ exonuclease or only a 5′ exonuclease may be used), is used to remove sequestering agents and unligated complementary nucleic acids. For example, in some embodiments, the first and second complementary nucleic acids may be protected from digestion by including phosphorothioate linkages at the 3′ and 5′ ends distal to the ligation site. A rolling circle probe containing a sequence complementary to the first complementary nucleic acid at one of its termini and a sequence complementary to the second complementary nucleic acid at the other terminus is hybridized to the ligation product. The rolling circle probe also contains a sequence complementary to a tag to be used to indicate the presence of the target nucleic acid in the sample. The rolling circle probe hybridized to the ligation product is ligated to generate a circular molecule. DNA polymerase is provided to extend the ligation product which is hybridized to the circular molecule, generating a product containing repeating units comprising the tag. The product may be trimmed and placed in contact with a detector comprising a detection probe complementary to the tag and hybridization is detected as described above.

It will be appreciated that, if desired, rather than utilizing both a first and second sequestering agent as described above, one may use only a single sequestering agent. If only a single sequestering agent is used, one may use either the first or second sequestering agent described above.

Although not illustrated in FIG. 13, it will be appreciated that, if desired, similar to the approach outlined above with respect to FIG. 3, both alleles of the SNP may be detected using a single mixture. In such embodiments, three complementary nucleic acids are used. The first complementary nucleic acid is complementary to the sequence 5′ of the polymorphic nucleotide in the genomic DNA. The second complementary nucleic acid includes a first allele of the polymorphic nucleotide at its 3′ terminus as well as a sequence complementary to the sequence 3′ of the polymorphic nucleotide in the genomic DNA. The third complementary nucleic acid includes a second allele of the polymorphic nucleotide at its 3′ terminus as well as a sequence complementary to the sequence 3′ of the polymorphic nucleotide in the genomic DNA. Two RC probes, each containing sequences complementary to the first complementary nucleic acid and to either the second complementary nucleic acid or the third complementary nucleic acid are also provided. The first, second and third complementary nucleic acids and the two RC probes are mixed together and placed in contact with the genomic DNA.

For example, in one embodiment, the same first complementary nucleic acid may be used to detect each of the alleles of the SNP. The sequestering agents and first, second and third complementary nucleic acids are placed in contact with the genomic DNA. If the sample contains both alleles of the SNP, two ligation products will be generated. One ligation product comprises the first complementary nucleic acid ligated to the second complementary nucleic acid. The other ligation product comprises the first complementary nucleic acid ligated to the third complementary nucleic acid. Rolling circle amplification is performed as described above and the resulting products comprise tags indicative of the presence of the corresponding alleles of the SNP which are present in the sample. The products are detected as described above. In this way, both alleles of the SNP may be assayed using a single mixture.

In some embodiments, both alleles of a large number of SNPs are simultaneously assayed in a single mixture by using complementary nucleic acids complementary to each allele of each SNP and appropriate sequestering agents and RC probes as described above. In such multiplexed reactions, for each SNP the corresponding RC probe contains a sequence complementary to a unique tag indicative of the presence of a particular allele of a SNP in the sample.

### Example 11

### Complementary Nucleic Acids and Sequestering Agents for Detection of SNP 1698 of the F5 Gene

The F5 gene encodes human coagulation factor V. SNP 1698 of the F5 gene has two alleles, a G allele and an A allele. Presence of the A allele is associated with a clotting disorder.

The genomic sequence of the F5 gene near position 1698 is:

TCTGTAAGAGCAGATCCCTGGACAGGCAAGGAATACAGAGGGCAGCAGAC
ATCG (SEQ ID NO: 1, with the A allele shown in
bold)

A first complementary nucleic acid of SEQ ID NO: 2 below and a second complementary nucleic acid of SEQ ID NO: 3 below may be used in any of the above methods to detect the presence of the A allele in a sample.

SEQ ID NO: 2: GCCTGTCCAGGGATCTGCTCTTACAGA
SEQ ID NO: 3 GGACAAAATACCTGTATTCCTT

The sequestering agents of SEQ ID NO: 4 below and SEQ ID NO: 5 below may be used in any of the above methods to detect the presence of the A allele in a sample.

```
SEQ ID NO: 4: GATCCCTGGACAGGCCGGAAGCGGCTTTTTTGCCGC
                            TTCCGdd (Gdd = dideoxyG)
SEQ ID NO: 5 GTGCCGAGACGTTTTTTCGTCTCGGCACTAGGAATA
                            CAGGT
```

FIG. 14 illustrates the structure of duplexes between the complementary nucleic acids and the sequestering agents. As illustrated in FIG. 14, the duplex between the first complementary nucleic acid and the first sequestering agent has a Tm of 77.2° C. while the duplex between the first complementary nucleic acid and the target has a Tm of 87.1° C. The hairpin loop in the first sequestering agent has a Tm of 81.0° C. Likewise, as illustrated in FIG. 15, the duplex between the second complementary nucleic acid and the second sequestering agent has a Tm of 64.2° C. while the duplex between the second complementary nucleic acid and the target has a Tm of 75.4° C. The hairpin loop in the second sequestering agent has a Tm of 81.0° C. The complementary nucleic acids of SEQ ID NO: 2 and SEQ ID NO: 3 and the sequestering agents of SEQ ID NO: 4 and SEQ ID NO: 5 may be used in any of the methods described herein to detect the presence of the A allele in a sample.

In some embodiments it may be desirable that the duplex between the target and the first and second complementary nucleic acids have comparable stability. This embodiment allows the duplex between the allele-specific complementary nucleic acid and the allele-containing target to play a thermodynamic role in aiding the specificity of the overall ligation process. Sequences reflecting this embodiment are shown in FIGS. 16A and 16B. It will be appreciated that if desired one may modify the nucleotides at the 5′ and/or 3′ ends of the sequestering agents depicted in FIGS. 16A and 16B as described herein to reduce or eliminate ligation of the complementary nucleic acids to the sequestering agents. In other embodiments, the duplex between the allele-specific second complementary nucleic acid and the target may have a higher stability than the duplex between the target and the first complementary nucleic acid. In further embodiments, the duplex between the allele-specific second complementary nucleic acid and the target may have a lower stability than the duplex between the target and the first complementary nucleic acid.

### Example 12

### Complementary Nucleic Acids and Sequestering Agents for Detection of the Alleles of the MTHFR Gene Present in a Sample

The MTHFR gene encodes methylene tetrahydrofolate reductase., an enzyme which catalyzes the remethylation of hymocysteine to methionine. A C-to-T transition in exon 4 of the MTHFR gene converts an alanine to a valine residue, resulting in elevated levels of plasma homocysteine associated with an increased risk of coronary artery disease.

A first complementary nucleic acid which may be used in any of the methods described herein and which hybridizes immediately upstream of the polymorphic nucleotide has the sequence CTCCCGCAGACACCTTCTCCTTCAAG (SEQ ID NO: 6). A second complementary nucleic acid which may be used in any of the methods described herein and which hybridizes immediately adjacent to the first nucleic acid and which has a G complementary to the C allele of the MTHFR gene at its 3′ end has the sequence TGATGATGAAATCGG (SEQ ID NO: 7). A third complementary nucleic acid which may be used in any of the methods described herein and which hybridizes immediately adjacent to the first complementary nucleic acid and which has an A complementary to the T allele of the MTHFR gene at its 3′ end has the sequence TGATGATGAAATCGA (SEQ ID NO: 8).

A first sequestering agent which comprises a sequence complementary to a portion of the first complementary nucleic acid and which may be used in any of the methods described herein has the sequence GGTGTCTGCGGGAGCGGAAGCGGCTTTTTGCCGCTTCCGdd (SEQ ID NO: 9). The first sequestering agent comprises a dideoxyG at its 3′ end to prevent ligation to the 5′ phosphate of the first complementary nucleic acid when the first complementary nucleic acid is hybridized thereto.

A second sequestering agent which comprises a sequence complementary to a portion of the second complementary nucleic acid and which may be used in any of the methods described herein has the sequence GCTGCACCGCTTTTTTGCGGTGCACCCGATTTCATCA (SEQ ID NO: 10). The bold C in SEQ ID NO: 10 is the polymorphic nucleotide in the C allele of the MTHFR gene.

A third sequestering agent which comprises a sequence complementary to a portion of the third complementary nucleic acid and which may be used in any of the methods described herein has the sequence GCTGCACCGCTTTTTTGCGGTGCACTCGATTTCATCA (SEQ ID NO: 11). The bold T in SEQ ID NO: 11 is the polymorphic nucleotide in the T allele of the MTHFR gene.

The complementary nucleic acids of SEQ ID NOs 6-8 and the sequestering agents of SEQ ID NOs: 9-11 may be used in any of the methods described herein to detect the presence of the C and/or T alleles in a sample.

### Example 13

### RC probe for Detecting p253SNP3

A linear DNA molecule suitable for use as an RC padlock probe is designed to hybridize to a single nucleotide polymorphism in the p53 gene known as p53 SNP3. The RC probe (SEQ ID NO: 12) contains sequence complementary to the target nucleotide sequence for the wild-type variant of p53 SNP3. Included in the probe are the following: a tag sequence known as the Z′ tag (or, Z′ “zipcode”) (SEQ ID NO: 13), a T7 RNA polymerase promoter (SEQ ID NO: 14), an Eco RI restriction endonuclease site (SEQ ID NO: 15), and a 3′ nucleotide gap (SEQ ID NO: 16) to aid T7 transcription. Sequence complementary to p53 SNP3 target nucleotide sequence is located on the 3′ end (SEQ ID NO: 17) and 5′ end (SEQ ID NO: 18) of SEQ ID NO: 8, with a 24-nucleotide sequence on the 5′ end and a 13-nucleotide sequence on the 3′ end. The “backbone” sequence of the RC probe containing non-target-complementary sequences is compared against the human genome and no comparable matches are found between the padlock probe backbone and sequences in the human genome.

| RC probe for p53 SNP3, using Z′ tag | | |
|---|---|---|
| 5′end: | GCACCTCAAAGCTGTTCCGTCCCA | (SEQ ID NO: 18) |
| | Tm = 65.2° C.; 24-mer | |
| 3′end: | CAGGCACAAACAC | (SEQ ID NO: 17) |
| | Tm = 42.0° C.; 13-mer | |
| Z′ tag: | AGCTACTGGCAATCT | (SEQ ID NO: 13) |
| T7 promoter: | CCCTATAGTGAGTCGTATTA | (SEQ ID NO: 14) |
| Eco RI site: | GAATTC | (SEQ ID NO: 15) |
| 3-nucleotide | GAT | (SEQ ID NO: 16) |
| gap, helps | | |
| transcription | | |
| of T7 | | |
| polymerase: | | |
| RC primer: | GATAGGAGTCAGTTAAGATCG | (SEQ ID NO: 19) |
| Entire RC | 5′P GCACCTCAAAGCTGTTCCGT | (SEQ ID NO: 12) |
| probe: | CCCAGTTGACTATCCTCAGTGAAT | |
| | TCTAGCTACTGGCAATCTGATCCC | |
| | TATAGTGAGTCGTATTACAGGCAC | |
| | AAACAC | |

where the features of the RC probe (SEQ ID NO: 12) are identified as follows:

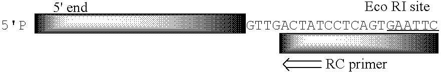

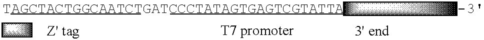

The rolling circle probe may be used in any of the methods described herein.

### Example 14

### Ruthenium Detection of Products Bound to Carbon Ink Electrodes

Immobilization of detection probe on universal chips:

Immobilization of Streptavidin or NeutrAvidin on the chips: NeutrAvidin is dissolved in 10 mM HEPES/10 mM LiCl, pH 7.4 buffer containing 25% isopropyl alcohol. The NeutrAvidin solution at concentrations of 40 to 4,000 nM is deposited on the surface of working electrodes on a universal chip and allowed to dry completely at room temperature. StabilCoat solution, a solution to stabilize biomolecules, is added to the working electrodes on the universal chip and allowed to incubate for 10 minutes. The Stabilicoat solution is aspirated from the universal chip surface and the chip is dried briefly.

Binding of Detection Probe on NeutrAvidin Immobilized Universal Chip:

A detection probe which is biotinylated at the 5′ end and contains a tag sequence complementary to the tag sequence indicative of the presence of a target nucleic acid in a sample generated using any of the methods described above, is incubated with a NeutrAvidin immobilized electrode surface at room temperature for 30 minutes. The biotinylated DNA solution is removed and the chip is washed by immersion in 10 mM HEPES/10 mM LiCl, pH 7.4 buffer. The chip with immobilized detection probes can be coated with a thin layer of Stabilcoat for storage.

Hybridization of Target Nucleic Acids to Detection Probe Immobilized on a Universal Array:

A nucleic acid comprising a tag sequence indicative of the presence of a target nucleic acid in a sample is obtained using any of the methods described above. The nucleic acid comprising the tag is applied to a universal chip comprising a detection probe with a sequence complementary to the tag and allowed to hybridize to the detection probe at room temperature for 30 minutes. Hybridization conditions such as salt concentration, the pH, incubation time, and temperature can be varied by one of skill in the art to optimize binding.

Electrochemical Measurement of Immobilized DNA Target:

DNA immobilized on the universal chip is detected using square wave voltammetry. Other methods such as cyclic voltammetry, differential pulse voltammetry can also be used. Rutheniumhexamine, Ru(NH₃)₆³⁺, is a preferred cationic redox reporter for the detection of immobilized DNA on a universal chip. Square wave voltammetry for the detection of Ru(NH₃)6³⁺ associated with surface DNA target is performed as follows. A three-electrode system is used: a silver wire reference electrode, a platinum wire auxiliary electrode and universal chip comprising carbon ink working electrodes with captured detection probes which may be sequence complementary to tag sequence, or may be DNA target nucleotide sequence. The chip is immersed in aqueous buffer containing 5 μM Ru(NH₃)₆³⁺, 10 mM Tris/10 mM NaCl, pH 7.4 in an electrochemical cell. Square wave voltammograms are recorded after scanning from 0 to −500 mV at conditions of 25 mV amplitude, 4 mV step potential and 15 Hz frequency. The parameters of the square wave voltammetry can be varied as would be clear to those of skill in the art.

### Example 15

### Transcription of Ligation Products

Polymerase chain reaction (PCR) is performed on a target region containing a single nucleotide polymorphism in the p53 gene known as p53 SNP 3. First and second complementary nucleic acids and first and second sequestering agents complementary to portions of the first and second complementary nucleic acids respectively are provided. The first and second complementary nucleic acids are then hybridized to the PCR product. The first complementary nucleic acid contains a target-specific region on the 5′ end, a T7 promoter region on the 3′ end and a 3′ biotin label. The second complementary nucleic acid contains a target-specific region on the 3′ end and a tag sequence Z′ on the 5′ end. If the sample contains the p53SNP3 allele which is complementary to the polymorphic nucleotide included in the second complementary nucleic acid, a ligation product is formed thereby linking the T7 promoter region and the tag sequence Z′. The ligation product is captured by incubating the mixture with particles coated with streptavidin, and subsequently isolating the particles and discarding the supernatant such that excess second complementary nucleic acid is washed away with the supernatant. A promoter oligonucleotide is then added which hybridizes to the promoter sequence in the ligation product, producing a double-stranded site on which transcription is initiated with the addition of the RNA polymerase. Transcription of the ligation product produces multiple copies of the complement of the Z′ tag sequence. These multiple copies of the Z′ complement are then exposed to a universal chip for hybridization to the Z′ tag (detection probe) on the chip. Hybridization of the tag sequence to a complementary detection probe is measured by electrochemical detection of Ru(III) complex bound electrostatically to phosphodiesters, as described above.

### Example 16

### PCR Products Tethered to a Bead

A 560 base pair (bp) region of the p53 gene is amplified by PCR using a forward primer containing a biotin label on the 5′ end. The resulting double-stranded 560 bp PCR product contains single nucleotide polymorphisms (SNPs) 1, 2, and 3. The double-stranded product is denatured and the bottom strand is washed away, resulting in several copies of the single-stranded 560 nucleotide region. The three relevant padlock probes (70.24, 70.31, and 70.33 containing tag sequences X′, Y′, and Z′ respectively) and sequestering agents containing sequences complementary to portions of the padlock probes are provided. Allele specific ligation of the padlock probes is performed. The PCR products with hybridized padlock probes containing a 5′ Biotin label are captured with Streptavidin beads and the unhybridized padlock probes are washed away. Linear RC amplification is then performed simultaneously on the three padlocks, producing multiple length RCA products containing complements to the tag sequences X′ (SNP 1), Y′ (SNP 2), and Z′ (SNP 3). These RC amplification products are exposed to a universal chip for hybridization to the tag sequences on the chip. The hybridization of the amplification product is read out by electrochemical detection of Ru(III) complex bound electrostatically to phosphodiesters.

1. A method for detecting the presence of a target nucleic acid in a sample comprising:
providing a sample comprising a target nucleic acid and non-target nucleic acids;
providing a first complementary nucleic acid which is complementary to a first nucleotide sequence in a target nucleic acid;
providing a second complementary nucleic acid which is complementary to a second nucleotide sequence in said target nucleic acid;
providing a first sequestering agent which specifically interacts with said first complementary nucleic acid, wherein said first sequestering agent reduces the likelihood that said first complementary nucleic acid will hybridize to said non-target nucleic acids;
hybridizing said first complementary nucleic acid to said first nucleotide sequence in said target nucleic acid;
hybridizing said second complementary nucleic acid to said second nucleotide sequence in said target nucleic acid;
ligating said first complementary nucleic acid which is hybridized to said first nucleotide sequence in said target nucleic acid to said second complementary nucleic acid which is hybridized to said second nucleotide sequence in said target nucleic acid, thereby forming a ligation product; and
detecting the presence of said ligation product.

2. The method of claim 1, wherein said first complementary nucleic acid and said second complementary nucleic acid are present on the same nucleic acid molecule such that said ligation product comprises a circular nucleic acid.

3. The method of claim 1, wherein a first oligonucleotide comprises said first complementary nucleic acid and a second distinct oligonucleotide comprises said second complementary nucleic acid.

4. The method of claim 1, wherein said first nucleotide sequence in said target nucleic acid and said second nucleotide sequence in said target nucleic acid are immediately adjacent to one another on said target nucleic acid.

5. The method of claim 1, wherein said first nucleotide sequence in said target nucleic acid and said second nucleotide sequence in said target nucleic acid are not immediately adjacent to one another on said target nucleic acid.

6. The method of claim 1 further comprising providing a second sequestering agent which interacts with said second complementary nucleic acid, thereby reducing the likelihood that said second oligonucleotide will hybridize to said non-target nucleic acids.

7. The method of claim 1, wherein said first sequestering agent comprises a nucleic acid comprising a nucleotide sequence which is complementary to at least a portion of said first complementary nucleic acid, wherein a duplex between said first complementary nucleic acid and said sequestering agent has a melting point intermediate to the melting point of a duplex between said first complementary nucleic acid and said non-target nucleic acids and the melting point of a duplex between said first complementary nucleic acid and said first nucleotide sequence in said target nucleic acid.

8. The method of claim 7, wherein the residue at the 3′ terminus of said first sequestering agent lacks a 3-hydroxyl group such that the 5′ phosphate of said first complementary nucleic acid cannot be ligated to the 3′ terminus of said sequestering agent.

9. The method of claim 8, wherein the residue at the 3′ terminus of said first sequestering agent comprises a dideoxysugar.

10. The method of claim 8, wherein the residue at the 3′ terminus of said sequestering agent comprises a blocking group such that the 5′ phosphate of said first complementary nucleic acid cannot be ligated to the 3′ terminus of said first sequestering agent.

11. The method of claim 10, wherein said blocking group is selected from the group consisting of NH₂, F, Cl, Br, NO₂, OR₁, O—C(O)—R₂, NHR₃, alkyl, and H where R₁ is selected from the group consisting of carbonyl, alkyl, acyl, alkene, or heteroalkyl, said alkyl, acyl, alkene or heteroalkyl group being substituted or unsubstituted, R₂ is selected from the group consisting of alkyl, acyl, alkene, or heteroalkyl, said alkyl, acyl, alkene or heteroalkyl group being substituted or unsubstituted and R3 is selected from the group consisting of alkyl, acyl, alkene, or heteroalkyl, said alkyl, acyl, alkene or heteroalkyl group being substituted or unsubstituted.

12. The method of claim 8, wherein said sequestering agent comprises a stem loop.

13. The method of claim 12, wherein the 3′ terminus is in the duplex portion of said stem loop.

14. The method of claim 7, wherein said sequestering agent has a linear conformation.

15. The method of claim 7, wherein said sequestering agent reduces the accessibility of the 5′ phosphate of said first complementary nucleic acid to a ligase when said first complementary nucleic acid is hybridized to said sequestering agent.

16. The method of claim 7, wherein said sequestering agent lacks a 5′ phosphate.

17. The method of claim 16, wherein the nucleotide at the 5′ terminus of said sequestering agent comprises a blocking group which prevents said nucleotide from being ligated to another nucleic acid, wherein said blocking group is selected from the group consisting of NH₂, F, Cl, Br, NO₂, OR₁, O—C(O)—R₂, NHR₃, alkyl, and H where R₁ is selected from the group consisting of carbonyl, alkyl, acyl, alkene, or heteroalkyl, said alkyl, acyl, alkene or heteroalkyl group being substituted or unsubstituted, R₂ is selected from the group consisting of alkyl, acyl, alkene, or heteroalkyl, said alkyl, acyl, alkene or heteroalkyl group being substituted or unsubstituted and R3 is selected from the group consisting of alkyl, acyl, alkene, or heteroalkyl, said alkyl, acyl, alkene or heteroalkyl group being substituted or unsubstituted.

18. The method of claim 7, wherein said sequestering agent reduces the accessibility of the 3′ hydroxyl of said first complementary nucleic acid to a ligase when said first complementary nucleic acid is hybridized to said sequestering agent.

19. The method of claim 6, wherein said first equestering agent reduces the accessibility of the 5′ phosphate of said first complementary nucleic acid to a ligase when said first complementary nucleic acid is hybridized to said first sequestering agent and wherein said second sequestering agent reduces the accessibility of the 3′ hydroxyl of said second complementary nucleic acid to a ligase when said second complementary nucleic acid is hybridized to said second sequestering agent.

20. The method of claim 1, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid.

21. The method of claim 6, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid and wherein said hybridizing step comprises hybridizing said second complementary nucleic acid to said second nucleotide sequence in said target nucleic acid at a temperature selected from the group consisting of a temperature at least 5 degrees Celsius below the melting temperature of the duplex between said second complementary nucleic acid and said second nucleotide sequence in said target nucleic acid, a temperature at least 10 degrees Celsius below the melting temperature of the duplex between said second complementary nucleic acid and said second nucleotide sequence in said target nucleic acid, a temperature at least 15 degrees Celsius below the melting temperature of the duplex between said second complementary nucleic acid and said second nucleotide sequence in said target nucleic acid, a temperature at least 20 degrees Celsius below the melting temperature of the duplex between said second complementary nucleic acid and said second nucleotide sequence in said target nucleic acid, and a temperature at least 25 degrees Celsius below the melting temperature of the duplex between said second complementary nucleic acid and said second nucleotide sequence in said target nucleic acid.

22. The method of claim 7, wherein said target nucleic acid comprises a single nucleotide polymorphism.

23. The method of claim 22, wherein said first complementary nucleic acid comprises the variable nucleotide of said single nucleotide polymorphism such that said first complementary nucleic acid is fully complementary to a first allele of said single nucleotide polymorphism.

24. The method of claim 23, wherein said variable nucleotide position is at the 3′ terminus of said first complementary nucleic acid.

25. The method of claim 23, further comprising:
providing a third complementary nucleic acid wherein said third complementary nucleic acid comprises the variable nucleotide of said single nucleotide polymorphism such that said third complementary nucleic acid is fully complementary to a second allele of said single nucleotide polymorphism,
providing a second sequestering agent comprising a nucleic acid which is complementary to at least a portion of said third complementary nucleic acid and wherein said at least a portion of said third complementary nucleic acid includes said variable nucleotide of said second allele of said single nucleotide polymorphism;
wherein said at least a portion of said first complementary nucleic acid to which said first sequestering agent is complementary includes said variable nucleotide of said first allele of said single nucleotide polymorphism.

26. The method of claim 1, wherein said sequestering agent is present in excess of said first complementary nucleic acid.

27. The method of claim 26, wherein said sequestering agent is present in a molar excess of about 1.5 to about 2 relative to said first complementary nucleic acid.

28. The method of claim 1, wherein the concentrations of said first complementary nucleic acid and said sequestering agent are selected so that at equilibrium about 5% or less of said first nucleotide sequence in said target nucleic acid is unoccupied by said first complementary nucleic acid.

29. The method of claim 1, wherein said sample comprises genomic DNA.

30. The method of claim 1, wherein said step of detecting the presence of said ligation product comprises performing a PCR reaction to amplify said ligation product, thereby generating an amplified ligation product and detecting said amplified ligation product.

31. The method of claim 1, wherein said step of detecting the presence of said ligation product comprises circularizing a nucleic acid template, thereby generating a circular molecule if said ligation product is present and detecting said circular molecule.

32. The method of claim 1, wherein said step of detecting the presence of said ligation product comprises performing a rolling circle amplification reaction, thereby generating an amplification product and detecting said amplification product.

33. The method of claim 1, wherein said step of detecting the presence of said ligation product comprises detecting hybridization of said ligation product or of a nucleic acid indicative of the presence of said ligation product to a solid support.

34. The method of claim 33, wherein said nucleic acid indicative of the presence of said ligation product comprises an identifier tag wherein said identifier tag is generated only when said target nucleotide sequence corresponding to said identifier tag is present in said sample.

35. The method of claim 33, wherein said solid support comprises a universal detector.

36. The method of claim 35, wherein said universal detector comprises detection probes coupled to a detecting component which measures hybridization of said ligation product or of said nucleic acid indicative of the presence of said ligation product to any of said detection probes.

37. The method of claim 36, wherein said detecting component is electrochemical, fluorescent, calorimetric, radiometric or magnetic.

38. The method of claim 37, wherein said detecting component is electrochemical.

39. The method of claim 36, wherein said detection probes coupled to said detecting component are attached to a surface, film, or particle.

40. The method of claim 39, wherein said detection probes are attached to said surface, film, or particle by covalent bonds, ionic bonds, electrostatic interactions or adsorptive interactions.

41. The method of claim 39, wherein said detection probes are attached to a particle.

42. The method of claim 39, wherein said detection probes are attached to a plurality of particles.

43. The method of claim 35, wherein said universal detector comprises an array of detection probes coupled to detecting components, said array comprising electrodes attached to a substrate.

44. The method of claim 43, wherein said electrodes are gold or carbon.

45. The method of claim 44, wherein said electrodes are gold.

46. The method of claim 43, further comprising measuring hybridization to any said detection probe by ruthenium amperometry.

47. The method of claim 43, wherein said electrodes are coated with protein which can be bound by oligonucleotides derivatized with a moiety that binds said protein that coats said electrode.

48. The method of claim 47, wherein said electrodes are coated with avidin such that said electrodes can be bound by biotin-labeled oligonucleotides.

49. An isolated nucleic acid comprising:
a first nucleotide sequence complementary to a probe nucleic acid such that said probe nucleic acid can hybridize to said nucleic acid, wherein at least a portion of said probe nucleic acid is complementary to a target nucleic acid;
a second nucleotide sequence comprising a region which forms a structure such that one terminus of said nucleic acid is in proximity to one terminus of said probe nucleic acid when said probe nucleic acid is hybridized to said nucleic acid and wherein a duplex between said probe nucleic acid and said first nucleotide sequence has a melting point intermediate to the melting point of a duplex between said probe nucleic acid and said target nucleic acid and the melting point of a duplex between said probe nucleic acid and non-target nucleic acids.

50. The nucleic acid of claim 49, wherein said nucleic acid reduces the accessibility of the 5′ phosphate of said probe nucleic acid to a ligase when said probe nucleic acid is hybridized to said nucleic acid.

51. The nucleic acid of claim 49, wherein said nucleic acid lacks a 5′ phosphate.

52. The nucleic acid of 51, wherein the nucleotide at the 5′ terminus of said nucleic acid comprises a blocking group which prevents said nucleotide from being ligated to another nucleic acid, wherein said blocking group is selected from the group consisting of NH₂, F, Cl, Br, NO₂, OR₁, O—C(O)—R₂, NHR₃, alkyl, and H where R₁ is selected from the group consisting of carbonyl, alkyl, acyl, alkene, or heteroalkyl, said alkyl, acyl, alkene or heteroalkyl group being substituted or unsubstituted, R₂ is selected from the group consisting of alkyl, acyl, alkene, or heteroalkyl, said alkyl, acyl, alkene or heteroalkyl group being substituted or unsubstituted and R3 is selected from the group consisting of alkyl, acyl, alkene, or heteroalkyl, said alkyl, acyl, alkene or heteroalkyl group being substituted or unsubstituted.

53. The nucleic acid of claim 49, wherein the terminus of said nucleic acid which is adjacent to said probe nucleic acid when said probe nucleic acid is hybridized to said nucleic acid contains a moiety thereon which cannot be ligated to said probe nucleic acid by a ligase.

54. The nucleic acid of claim 53, wherein said moiety which cannot be ligated to said probe nucleic acid by a ligase is on the residue at the 3′ terminus of said nucleic acid.

55. The nucleic acid of claim 54, wherein the 3′ terminus of said nucleic acid lacks a 3-hydroxyl group such that the 5′ phosphate of said probe nucleic acid cannot be ligated to the 3′ terminus of said nucleic acid.

56. The nucleic acid of claim 55, wherein the residue at the 3′ terminus of said nucleic acid comprises a dideoxysugar.

57. The nucleic acid of claim 55, wherein the residue at the 3′ terminus of said nucleic acid comprises a blocking group such that the 5′ phosphate of said probe nucleic acid cannot be ligated to the 3′ terminus of said nucleic acid.

58. The nucleic acid of claim 57, wherein said blocking group is selected from the group consisting of NH₂, F, Cl, Br, NO₂, OR₁, O—C(O)—R₂, NHR₃, alkyl, and H where R₁ is selected from the group consisting of carbonyl, alkyl, acyl, alkene, or heteroalkyl, said alkyl, acyl, alkene or heteroalkyl group being substituted or unsubstituted, R₂ is selected from the group consisting of alkyl, acyl, alkene, or heteroalkyl, said alkyl, acyl, alkene or heteroalkyl group being substituted or unsubstituted and R3 is selected from the group consisting of alkyl, acyl, alkene, or heteroalkyl, said alkyl, acyl, alkene or heteroalkyl group being substituted or unsubstituted.

59. The nucleic acid of claim 49, wherein said structure comprises a stem loop.

60. The nucleic acid of claim 59, wherein the 3′ terminus of said nucleic acid is in the duplex portion of said stem loop.

61. The nucleic acid of claim 49, wherein said first nucleotide sequence which is complementary to at least a portion of said probe nucleic acid comprises a sequence which is polymorphic in the genome of an organism containing said target nucleic acid.

62. The nucleic acid of claim 61, wherein said first nucleotide sequence which is complementary to at least a portion of said probe nucleic acid comprises the variable nucleotide of a single nucleotide polymorphism in the genome of an organism containing said target nucleic acid.

63. The nucleic acid of claim 62, wherein said variable nucleotide is complementary to the 3′ terminus of said probe nucleic acid.

64-94. (canceled)

95. A method for determining whether a target nucleic acid is present in a sample comprising:
providing a sample to be tested for the presence of a target nucleic acid said sample comprising non-target nucleic acids;
providing a probe comprising a first end comprising a first complementary nucleic acid complementary to a first nucleotide sequence in said target nucleic acid, a second end comprising a second complementary nucleic acid complementary to a second nucleotide sequence in said target nucleic acid, a promoter recognized by an RNA polymerase, and a nucleotide sequence complementary to a tag sequence indicative of the presence of said target nucleic acid in said sample;
providing a first sequestering agent which sepecifically interacts with said first complementary nucleic acid, wherein said first sequestering agent reduces the likelihood that said first complementary nucleic acid will hybridize to said non-target nucleic acids;
contacting said sample with said probe, such that said first complementary nucleic acid will hybridize to said first nucleotide sequence in said sample and said second complementary nucleic acid will hybridize to said second nucleotide sequence in said sample and said probe will adopt a circular conformation if said target nucleic acid is present in said sample;
ligating the ends of said circular probe together if said circular probe is present;
performing a transcription reaction with said RNA polymerase, thereby generating a transcript comprising said tag if said sample contains said target nucleic acid; and
determining whether said transcript has been generated

96. The method of claim 95, wherein said RNA polymerase is selected from the group consisting of T7 RNA polymerase, R4 RNA polymerase, T3 RNA polymerase, E. coli RNA polymerase, SP6 RNA polymerase, RNA polymerase II and RNA polymerase III.

97. The method of claim 95, wherein said promoter is positioned 3′ of said first complementary nucleic acid and said nucleotide sequence complementary to said tag is positioned 5′ of said second complementary nucleic acid.

98. The method of claim 95 further comprising performing an exonuclease digestion after ligating the ends of said probe together.

99. The method of claim 95 further comprising providing a second sequestering agent which specifically interacts with said second complementary nucleic acid, wherein said second sequestering agent reduces the likelihood that said second complementary nucleic acid will hybridize to said non-target nucleic acids.

100. The method of claim 95, wherein said first sequestering agent reduces the accessibility of the 5′ phosphate of said first complementary nucleic acid to a ligase when said first complementary nucleic acid is hybridized to said first sequestering agent.

101. The method of claim 95, wherein said first sequestering agent reduces the accessibility of the 3′ hydroxyl of said first complementary nucleic acid to a ligase when said first complementary nucleic acid is hybridized to said sequestering agent.

102. The method of claim 99, wherein said first sequestering agent reduces the accessibility of the 5′ phosphate of said first complementary nucleic acid to a ligase when said first complementary nucleic acid is hybridized to said first sequestering agent and said second sequestering agent reduces the accessibility of the 3′ hydroxyl of said second complementary nucleic acid to a ligase when said second complementary nucleic acid is hybridized to said second sequestering agent.

103. The method of claim 95, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid.

104. The method of claim 99, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid and wherein said hybridizing step comprises hybridizing said second complementary nucleic acid to said second nucleotide sequence in said target nucleic acid at a temperature selected from the group consisting of a temperature at least 5 degrees Celsius below the melting temperature of the duplex between said second complementary nucleic acid and said second nucleotide sequence in said target nucleic acid, a temperature at least 10 degrees Celsius below the melting temperature of the duplex between said second complementary nucleic acid and said second nucleotide sequence in said target nucleic acid, a temperature at least 15 degrees Celsius below the melting temperature of the duplex between said second complementary nucleic acid and said second nucleotide sequence in said target nucleic acid, a temperature at least 20 degrees Celsius below the melting temperature of the duplex between said second complementary nucleic acid and said second nucleotide sequence in said target nucleic acid and a temperature at least 25 degrees Celsius below the melting temperature of the duplex between said second complementary nucleic acid and said second nucleotide sequence in said target nucleic acid.

105-128. (canceled)

129. A method for determining whether a target nucleic acid is present in a sample comprising:
providing a sample to be tested for the presence of a target nucleic acid said sample comprising non-target nucleic acids;
providing a first complementary nucleic acid comprising a promoter recognized by an RNA polymerase positioned 3′ of a sequence complementary to a first nucleotide sequence in said target nucleic acid;
providing a second complementary nucleic acid comprising a sequence complementary to a tag sequence indicative of the presence of said target nucleic acid in said sample positioned 5′ of a sequence complementary to a second nucleotide sequence in said target nucleic acid, wherein said second complementary nucleic acid is on a separate molecule from said first complementary nucleic acid;
providing a first sequestering agent which sepecifically interacts with said first complementary nucleic acid, wherein said first sequestering agent reduces the likelihood that said first complementary nucleic acid will hybridize to said non-target nucleic acids;
contacting said sample with said first complementary nucleic acid and said second complementary nucleic acid, such that said first complementary nucleic acid will hybridize to said first nucleotide sequence in said target nucleic acid and said second complementary nucleic acid will hybridize to said second nucleotide sequence in said target nucleic acid
ligating said first complementary nucleic acid to said second complementary nucleic acid if said target nucleic acid is present in said sample, thereby generating a ligation product if said target nucleic acid is present in said sample;
amplifying said ligation product if present;
performing a transcription reaction on said amplified ligation product with said RNA polymerase, thereby generating a transcript comprising said tag if said sample contains said target nucleic acid; and
determining whether said transcript has been generated.

130. The method of claim 129, wherein said RNA polymerase is selected from the group consisting of T7 RNA polymerase, R4 RNA polymerase, T3 RNA polymerase, E. coli RNA polymerase, SP6 RNA polymerase, RNA polymerase II and RNA polymerase III.

131. The method of claim 129, wherein said first complementary nucleic acid is resistant to digestion by a 3′ exonuclease and said second complementary nucleic acid is resistant to digestion by a 5′ exonduclease and wherein said method further comprises performing a digestion with a 3′ exonuclease and a 5′ exonuclease after ligating said first complementary nucleic acid to said second complementary nucleic acid if said sample contains said target nucleic acids.

132. The method of claim 129 further comprising providing a second sequestering agent which specifically interacts with said second complementary nucleic acid, wherein said second sequestering agent reduces the likelihood that said second complementary nucleic acid will hybridize to said non-target nucleic acids

133. The method of claim 129, wherein said first sequestering agent reduces the accessibility of the 5′ phosphate of said first complementary nucleic acid to a ligase when said first complementary nucleic acid is hybridized to said first sequestering agent.

134. The method of claim 132, wherein said first sequestering agent reduces the accessibility of the 5′ phosphate of said first complementary nucleic acid to a ligase when said first complementary nucleic acid is hybridized to said first sequestering agent and said second sequestering agent reduces the accessibility of the 3′ hydroxyl of said second complementary nucleic acid to a ligase when said second complementary nucleic acid is hybridized to said second sequestering agent.

135. The method of claim 129, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid.

136. The method of claim 132, wherein said hybridizing step comprises hybridizing said first complementary nucleic acid to said first nucleotide sequence in said target nucleic acid at a temperature selected from the group consisting of a temperature at least 5 degrees Celsius below the melting temperature of the duplex between said first complementary nucleic acid and said first nucleotide sequence in said target nucleic acid, a temperature at least 10 degrees Celsius below the melting temperature of the duplex between said first complementary nucleic acid and said first nucleotide sequence in said target nucleic acid, a temperature at least 15 degrees Celsius below the melting temperature of the duplex between said first complementary nucleic acid and said first nucleotide sequence in said target nucleic acid, a temperature at least 20 degrees Celsius below the melting temperature of the duplex between said first complementary nucleic acid and said first nucleotide sequence in said target nucleic acid and a temperature at least 25 degrees Celsius below the melting temperature of the duplex between said first complementary nucleic acid and said first nucleotide sequence in said target nucleic acid and wherein said hybridizing step comprises hybridizing said second complementary nucleic acid to said second nucleotide sequence in said target nucleic acid at a temperature selected from the group consisting of a temperature at least 5 degrees Celsius below the melting temperature of the duplex between said second complementary nucleic acid and said second nucleotide sequence in said target nucleic acid, a temperature at least 10 degrees Celsius below the melting temperature of the duplex between said second complementary nucleic acid and said second nucleotide sequence in said target nucleic acid, a temperature at least 15 degrees Celsius below the melting temperature of the duplex between said second complementary nucleic acid and said second nucleotide sequence in said target nucleic acid, a temperature at least 20 degrees Celsius below the melting temperature of the duplex between said second complementary nucleic acid and said second nucleotide sequence in said target nucleic acid and a temperature at least 25 degrees Celsius below the melting temperature of the duplex between said second complementary nucleic acid and said second nucleotide sequence in said target nucleic acid.

137-152. (canceled)

153. The method of claim 95 further comprising performing an amplification reaction on said sample, thereby amplifying said target nucleic acid if said target nucleic acid is present in said sample.

154. The method of claim 95 further comprising amplifying at least a portion of said probe comprising said promoter and said nucleotide sequence complementary to said tag.
